SEQUENCE LISTING (129 sequences)

SEQ ID NO: 1 - DNA, 17 bp, Homo sapiens
agaggtgsaacggaagc

SEQ ID NO: 2 - DNA, 17 bp, Homo sapiens
tccgggccsggagcagt

SEQ ID NO: 3 - DNA, 17 bp, Homo sapiens
aagggrccgcaatggag

SEQ ID NO: 4 - DNA, 18 bp, Homo sapiens
atactatcwtcatttact

SEQ ID NO: 5 - DNA, 17 bp, Homo sapiens
acaaawgaaagaacttg

SEQ ID NO: 6 - DNA, 13 bp, Homo sapiens
gggtgtaagtsag

SEQ ID NO: 7 - DNA, 14 bp, Homo sapiens
gatactggcccawa

SEQ ID NO: 8 - DNA, 17 bp, Homo sapiens
gcatwtgcaaatgcaag

SEQ ID NO: 9 - DNA, 17 bp, Homo sapiens
atctwgaagggtctgaa

SEQ ID NO: 10 - DNA, 18 bp, Homo sapiens
caggtggctctsgataag

SEQ ID NO: 11 - DNA, 17 bp, Homo sapiens
ctagaaggttsgggaag

SEQ ID NO: 12 - DNA, 20 bp, Homo sapiens
attttcagstgttgtctttg

SEQ ID NO: 13 - DNA, 19 bp, Homo sapiens
tgactatgcsaaagccaaa

SEQ ID NO: 14 - DNA, 19 bp, Homo sapiens
gtgggcagsagtggctgtg

SEQ ID NO: 15 - DNA, 22 bp, Homo sapiens
attgccatwgctcgtgcccttg

SEQ ID NO: 16 - DNA, 24 bp, Homo sapiens
cgcttgctaatmttattataagat

SEQ ID NO: 17 - DNA, 17 bp, Homo sapiens
gagcgcgggcscgagcg

SEQ ID NO: 18 - DNA, 17 bp, Homo sapiens
agcgcagcgcsggcccc

SEQ ID NO: 19 - DNA, 13 bp, Homo sapiens
gaagtcctsgggt

SEQ ID NO: 20 - DNA, 13 bp, Homo sapiens
attwtttaccaac

SEQ ID NO: 21 - DNA, 20 bp, Homo sapiens
gacccgatcmtacacctgct

SEQ ID NO: 22 - DNA, 21 bp, Homo sapiens
ctgcgccggaccgkggcgggt

SEQ ID NO: 23 - DNA, 18 bp, Homo sapiens
tcggggcgggsgccttca

SEQ ID NO: 24 - DNA, 17 bp, Homo sapiens
ctcagyttctgccattg

SEQ ID NO: 25 - DNA, 16 bp, Homo sapiens
ggccaggcwcgtggct

SEQ ID NO: 26 - DNA, 14 bp, Homo sapiens
cgggcttgbtggtg

SEQ ID NO: 27 - DNA, 16 bp, Homo sapiens
ccgggcttsgtggtgg

SEQ ID NO: 28 - DNA, 17 bp, Homo sapiens
cccakcgctttgggagg

SEQ ID NO: 29 - DNA, 14 bp, Homo sapiens
caggwaccacattt

SEQ ID NO: 30 - DNA, 14 bp, Homo sapiens
catttcasgtactt

SEQ ID NO: 31 - DNA, 14 bp, Homo sapiens
tgtggcaaktggct

SEQ ID NO: 32 - DNA, 13 bp, Homo sapiens
attggasaattgc

SEQ ID NO: 33 - DNA, 14 bp, Homo sapiens
tacatttycatttc

SEQ ID NO: 34 - DNA, 14 bp, Homo sapiens
tccascgcttggag

SEQ ID NO: 35 - DNA, 14 bp, Homo sapiens
gcagtttstttcag

SEQ ID NO: 36 - DNA, 14 bp, Homo sapiens
aagcgcwcagggac

SEQ ID NO: 37 - DNA, 14 bp, Homo sapiens
ccaactgcasatga

SEQ ID NO: 38 - DNA, 14 bp, Homo sapiens
ttcacggscaaggc

SEQ ID NO: 39 - DNA, 14 bp, Homo sapiens
aagtgggmtgcctg

SEQ ID NO: 40 - DNA, 14 bp, Homo sapiens
gcctggratgagct

SEQ ID NO: 41 - DNA, 14 bp, Homo sapiens
tggaatgwgctgaa

SEQ ID NO: 42 - DNA, 14 bp, Homo sapiens
taaataraagaatc

SEQ ID NO: 43 - DNA, 14 bp, Homo sapiens
aaatagwtaaataa

SEQ ID NO: 44 - DNA, 15 bp, Homo sapiens
ttagtctycattcac

SEQ ID NO: 45 - DNA, 15 bp, Homo sapiens
atcaasttagtcttc

SEQ ID NO: 46 - DNA, 15 bp, Homo sapiens
gatgcctrgaatgag

SEQ ID NO: 47 - DNA, 17 bp, Homo sapiens
gctgagctwcaaaggct

SEQ ID NO: 48 - DNA, 17 bp, Homo sapiens
gctgtgwctgaatgatg

SEQ ID NO: 49 - DNA, 18 bp, Homo sapiens
ctcagatsctctcaccta

SEQ ID NO: 50 - DNA, 18 bp, Homo sapiens
aggaggakgagcactctt

SEQ ID NO: 51 - DNA, 18 bp, Homo sapiens
gttgattttstcacctcc

SEQ ID NO: 52 - DNA, 18 bp, Homo sapiens
attcgggsgagctgaggc

SEQ ID NO: 53 - DNA, 18 bp, Homo sapiens
cggaggttgcrgtgagtt

SEQ ID NO: 54 - DNA, 18 bp, Homo sapiens
agactgasgtgggaggat

SEQ ID NO: 55 - DNA, 18 bp, Homo sapiens
tgtcctatttwtgaatgg

SEQ ID NO: 56 - DNA, 18 bp, Homo sapiens
atggtgaasaaactgtgg

SEQ ID NO: 57 - DNA, 18 bp, Homo sapiens
aaattgtsgaatacttct

SEQ ID NO: 58 - DNA, 18 bp, Homo sapiens
aggaattcwacatgcatg

SEQ ID NO: 59 - DNA, 18 bp, Homo sapiens
gtcaacaccraagataat

SEQ ID NO: 60 - DNA, 18 bp, Homo sapiens
aggcaaaawtatagtaaa

SEQ ID NO: 61 - DNA, 18 bp, Homo sapiens
tatagtaayagaaaccaa

SEQ ID NO: 62 - DNA, 18 bp, Homo sapiens
ataaaatastttttaggg

SEQ ID NO: 63 - DNA, 18 bp, Homo sapiens
tttattatasgtaaataa

SEQ ID NO: 64 - DNA, 19 bp, Homo sapiens
aattcatcwaactatatac

SEQ ID NO: 65 - DNA, 20 bp, Homo sapiens
agcctgaartataaacaaat

SEQ ID NO: 66 - DNA, 20 bp, Homo sapiens
aacaatagsataatggaatg

SEQ ID NO: 67 - DNA, 20 bp, Homo sapiens
aatggaatgtkaaaggaaaa

SEQ ID NO: 68 - DNA, 21 bp, Homo sapiens
aggaaaacraaccaatttaaa

SEQ ID NO: 69 - DNA, 21 bp, Homo sapiens
aggcttagtakgatctgctaa

SEQ ID NO: 70 - DNA, 21 bp, Homo sapiens
taactcagartcaggagtgtt

SEQ ID NO: 71 - DNA, 21 bp, Homo sapiens
aaggtcggyatttagctgaag

SEQ ID NO: 72 - DNA, 22 bp, Homo sapiens
cacccttcctyactcacttcct

SEQ ID NO: 73 - DNA, 21 bp, Homo sapiens
agaaaggcargacaaaatgaa

SEQ ID NO: 74 - DNA, 21 bp, Homo sapiens
ccaaaagtaakccaaaacaaa

SEQ ID NO: 75 - DNA, 22 bp, Homo sapiens
ccatgactrttttaagaggcta

SEQ ID NO: 76 - DNA, 24 bp, Homo sapiens
ttttagtttscttattctctctgt

SEQ ID NO: 77 - DNA, 20 bp, Homo sapiens
ttcagcctrgatgacagaac

SEQ ID NO: 78 - DNA, 24 bp, Homo sapiens
ctttgaaakttacagcattgtaga

SEQ ID NO: 79 - DNA, 20 bp, Homo sapiens
agtactgaacyggatgcaag

SEQ ID NO: 80 - DNA, 23 bp, Homo sapiens
tgcctggctrtgaaatttcattt

SEQ ID NO: 81 - DNA, 23 bp, Homo sapiens
cggatgtcctagaygcaggttat

SEQ ID NO: 82 - DNA, 24 bp, Homo sapiens
caagtttccrttcattttctgcat

SEQ ID NO: 83 - DNA, 24 bp, Homo sapiens
attcagcttcrtgttctctaacat

SEQ ID NO: 84 - DNA, 23 bp, Homo sapiens
aaaggggcatayatttataaaat

SEQ ID NO: 85 - DNA, 23 bp, Homo sapiens
caaggacataawatagagatatc

SEQ ID NO: 86 - DNA, 23 bp, Homo sapiens
agcttccaagmtcaaggaattct

SEQ ID NO: 87 - DNA, 23 bp, Homo sapiens
ccaaaataatrggtaatatatat

SEQ ID NO: 88 - DNA, 23 bp, Homo sapiens
tagaaagaagtagrgcattggcc

SEQ ID NO: 89 - DNA, 24 bp, Homo sapiens
tctccatctccartgagtattgag

SEQ ID NO: 90 - DNA, 24 bp, Homo sapiens
gcccaagsacataaatagagagat

SEQ ID NO: 91 - DNA, 24 bp, Homo sapiens
caaagagaactaraaattccatgt

SEQ ID NO: 92 - DNA, 25 bp, Homo sapiens
agtaaatgttctcyccttctgcaag

SEQ ID NO: 93 - DNA, 25 bp, Homo sapiens
gttttcctagarcctgttacttcat

SEQ ID NO: 94 - DNA, 22 bp, Homo sapiens
ccagggctttygtttattggga

SEQ ID NO: 95 - DNA, 24 bp, Homo sapiens
acaaaaattakccagtgtggtggt

SEQ ID NO: 96 - DNA, 24 bp, Homo sapiens
ccctggtagaakgtgcttgacaca

SEQ ID NO: 97 - DNA, 23 bp, Homo sapiens
gtgcagakagagtttgtggaatc

SEQ ID NO: 98 - DNA, 23 bp, Homo sapiens
gtgaccctgcttrggatacctat

SEQ ID NO: 99 - DNA, 23 bp, Homo sapiens
atcattcatccasccattcaccc

SEQ ID NO: 100 - DNA, 24 bp, Homo sapiens
tccctggggctycctgggaggctt

SEQ ID NO: 101 - DNA, 23 bp, Homo sapiens
agggaaatgtargtgtgaacagg

SEQ ID NO: 102 - DNA, 24 bp, Homo sapiens
acgcaatgggwgttttctccctcg

SEQ ID NO: 103 - DNA, 24 bp, Homo sapiens
gggagttttctycctcgagaatgt

SEQ ID NO: 104 - DNA, 24 bp, Homo sapiens
agggcacctcastaaagttctctt

SEQ ID NO: 105 - DNA, 24 bp, Homo sapiens
ttaaacaaatctargatcaggagt

SEQ ID NO: 106 - DNA, 24 bp, Homo sapiens
cctgtgccagakcacaatgtatct

SEQ ID NO: 107 - DNA, 24 bp, Homo sapiens
atcccaaggctctgarccctcaga

SEQ ID NO: 108 - DNA, 24 bp, Homo sapiens
tccacggcrtgtcatgaacatgtt

SEQ ID NO: 109 - DNA, 24 bp, Homo sapiens
ggcccacagggyactgctcccgtg

SEQ ID NO: 110 - DNA, 24 bp, Homo sapiens
agccccctgggkgctaagaacact

SEQ ID NO: 111 - DNA, 23 bp, Homo sapiens
gcaggacaraaaggatgatatat

SEQ ID NO: 112 - DNA, 24 bp, Homo sapiens
ggtcttgacgccyttccagatgct

SEQ ID NO: 113 - DNA, 23 bp, Homo sapiens
gaagagctgggwttggcctgtcc

SEQ ID NO: 114 - DNA, 23 bp, Homo sapiens
agtgtgcaggttamtgatgctgg

SEQ ID NO: 115 - DNA, 23 bp, Homo sapiens
actgggagggcmtggccggggct

SEQ ID NO: 116 - DNA, 24 bp, Homo sapiens
tttggactttaawcctatggaatg

SEQ ID NO: 117 - DNA, 22 bp, Homo sapiens
acagtttgggabttgaaatacg

SEQ ID NO: 118 - DNA, 25 bp, Homo sapiens
gagcagaaccccyccctggtccttc

SEQ ID NO: 119 - DNA, 752 bp, Homo sapiens
cctgcatcctgcacccccaggcatcccccctgcagcccccccagcatcccccctgcagcccccccagaacagggtgtttccccccctgcagcccccccagcatcccccctgcagcccccccagcatcccccctgcagcccccccagcatctcccctgcacccccagcatcccccctgcagcccttccagcatccccctgcacctctccaggatctccctgcaacccccattatcccccctgcacccctcgcagtatcccccctgcaccccccagcatccccccatgcaacccccggcatccagcattctccttgcaccctaccagtattcccccgcatcccggcccccctgcacccctccagcattctccttgcaccctaccagtattcccccgcatcccggcctccaagcctcccgcccaccttgcggtccccgccctggcgtctaggtggcaccagaatccctccaagcctcccgcccaccttgcggtccccgccctggcgtctaggtggcaccagaatcccgcgcggactccacccgctgggagctgccctcgcttgcccgtggttgtccagctcagtcccgcgcggactccacccgctgggagctgccctcgccggactccacccgctgggagctgccctcgcctccaagcctcccgcccaccttgcggtcccctaggtggcaccagaatccctccaagcctcccgcccaccttgcggtccccgccctggcgtctaggtggcacctcc

SEQ ID NO: 120 - DNA, 14 bp, Homo sapiens
  misc_feature (6)..(8): n is a, c, g, or t
ggtcannntgwcct

SEQ ID NO: 121 - DNA, 14 bp, Homo sapiens
ggtcasagaggacg

SEQ ID NO: 122 - DNA, 14 bp, Homo sapiens
ttttcgaccccccc

SEQ ID NO: 123 - DNA, 21 bp, Homo sapiens
aggagacctgrggttggagct

SEQ ID NO: 124 - DNA, 21 bp, Homo sapiens
agragacctgrggttggagct

SEQ ID NO: 125 - DNA, 16 bp, Homo sapiens
agaacatccctgtaca

SEQ ID NO: 126 - DNA, 15 bp, Homo sapiens
agaacattgggtttc

SEQ ID NO: 127 - DNA, 15 bp, Homo sapiens
agaacattttgtacg

SEQ ID NO: 128 - DNA, 15 bp, Homo sapiens
agaacagggtgttct

SEQ ID NO: 129 - DNA, 15 bp, Homo sapiens
agaacagggtgtttc

(19) United States
(12) Patent Application Publication
(10) Pub. No.: US 2014/0038836 A1
(43) Pub. Date: Feb. 6, 2014

(54) Novel Pharmacogene Single Nucleotide Polymorphisms and Methods of Detecting Same

(71) Applicant: AssureRx Health, Inc., Mason, OH (US)

(72) Inventors: Gerald A. Higgins, Takoma Park, MD (US); C. Anthony Altar, Mason, OH (US)

(21) Appl. No.: 13/904,792

(22) Filed: May 29, 2013

Related U.S. Application Data
(60) Provisional application No. 61/652,784, filed on May 29, 2012.

(51) Int. Cl. G06F 19/16; C12Q 1/68

(52) U.S. Cl. CPC G06F 19/16; C12Q 1/6827
USPC 506/9; 536/23.1; 435/320.1; 435/325; 435/6.11; 702/19

(57) ABSTRACT

The present invention provides pharmacogene polymorphisms and their use in predicting therapeutic effectiveness. The present invention also provides methods comprising targeted analysis of selected pharmacogenes in thousands of compiled whole human genome sequences for identifying polymorphic sequences associated with drug response are described. The methods also provide confirmation and validation of these pharmacogene polymorphisms, based on concordance between different sequencing technologies, and statistical error-checking. Imputation of the deleterious consequences of novel variants is predicted by bioinformatics analysis.

### INCORPORATION BY REFERENCE OF SEQUENCE LISTING

The contents of the text file named “42803_{—}504001US_ST25.txt”, which was created on Oct. 4, 2013 and is 21 KB in size, are hereby incorporated by reference in their entirety.

### BACKGROUND OF THE INVENTION

The effect of heredity on the responses of individuals to drugs is a topic of exceptional scientific interest. In the post-genomic era, researchers and clinicians are using human DNA sequence, genomic structures, human genetic variation, and changes in gene and protein expression, to more precisely define disease and develop new therapeutic interventions. Variations in genome sequence underlie differences in the way our bodies respond to drug treatment. The availability of thousands of whole human genomes now allows scientific researchers to detect novel variations in the genome that had not been previously discovered using other analytical methods.

There is great heterogeneity in the way individuals respond to medications, in terms of both host toxicity and treatment efficacy. There are many causes of this variability, including: severity of the disease being treated; drug interactions; and the individuals age and nutritional status. Despite the importance of these clinical variables, inherited differences in the form of genetic polymorphisms can have an even greater influence on the efficacy and toxicity of medications. Genetic polymorphisms in both drug-metabolizing enzymes (pharmacokinetic) and transporters, receptors, and other drug targets (pharmacodynamic) have been linked to inter-individual differences in the efficacy and toxicity of many medications.

Thus, there is a need in the art to identify new genetic polymorphisms to improve treatment outcome and for methods of more efficiently and effectively detecting these polymorphisms. The present invention addresses these needs.

### SUMMARY OF THE INVENTION

The present invention provides methods for interrogating thousands of aggregated whole human genome sequences, using targeted analysis of selected pharmacogenes, determining polymorphic sequences that may associate with drug response, executed on an inexpensive, energy-efficient, heterogeneous GPU-cluster based workstation.

The methods include aggregating populations of completed whole genome DNA sequences and performing a concordance check. The methods include scanning assembled whole human genomes for target enrichment of selected pharmacogenes, using genome browser coordinates for selected pharmacogenes based on user input. The methods include applying a multi-genome variant analysis algorithm to identify gene variants in said pharmacogenes, consisting of detection of novel single nucleotide polymorphisms (SNPs) and multi-nucleotide polymorphisms (MNPs), but not other structural variants, and apply statistical error-checking methods to validate SNPs and MNPs with allele frequencies of 0.1% to 99%.

The targeted, selected pharmacogenes had undetected nucleotide polymorphisms, including SNPs and MNPs. The ABCB1 gene contains 15 single nucleotide polymorphisms. The ADCYAP1R1 gene contains 5 single nucleotide polymorphisms and 1 multi-nucleotide polymorphism. The ADRA2A gene contains 2 single nucleotide polymorphisms and 1 multi-nucleotide polymorphism. The BDNF gene contains 2 single nucleotide polymorphisms. The COMT gene contains 3 single nucleotide polymorphisms. The CRHBP gene contains 5 single nucleotide polymorphisms. The CRHR1 gene contains 5 single nucleotide polymorphisms. The DBI gene contains 18 single nucleotide polymorphisms and 2 multi-nucleotide polymorphisms. The DRD2 gene contains 5 single nucleotide polymorphisms. The DRD4 gene contains 4 single nucleotide polymorphisms. The FKBP5 gene contains 10 single nucleotide polymorphisms. The GCR(NR3C1) gene contains 7 single nucleotide polymorphisms. The HTR2A gene contains 8 single nucleotide polymorphisms. The HTR2C gene contains 1 single nucleotide polymorphism and 2 multi-nucleotide polymorphisms. The NPY gene contains 2 single nucleotide polymorphisms. The NT3 gene contains 7 single nucleotide polymorphisms. The NTRK2 gene contains 10 single nucleotide polymorphisms. The OPRM1 gene contains 3 single nucleotide polymorphisms and 1 multi-nucleotide polymorphism. The SLC6A2 gene contains 2 single nucleotide polymorphisms and 2 multi-nucleotide polymorphisms. The SLC6A3 gene contains 12 single nucleotide polymorphisms. The SLC6A4 gene contains 10 single nucleotide polymorphisms and 1 multi-nucleotide polymorphism. The pharmacogene single nucleotide polymorphisms and multi-nucleotide polymorphisms are reported in a database.

The present invention provides a nucleic acid sequence comprising at least 10, at least 15 or at least 50 continuous nucleotides of the ABCB1 gene comprising at least one polymorphism of SEQ ID NOs: 1-15; of the ADCYAP1R1 gene comprising the polymorphism of SEQ ID NO: 16; of the ADRA2A gene comprising at least one polymorphism of SEQ ID NOs: 17-18; of the BDNF gene comprising at least one polymorphism of SEQ ID NOs: 19-20; of the COMT gene comprising at least one polymorphism of SEQ ID NOs: 21-23; of the CRHBP gene comprising the polymorphism of SEQ ID NO: 24; of the CRHR1 gene comprising at least one polymorphism of SEQ ID NOs: 25-28; of the DBI gene comprising at least one polymorphism of SEQ ID NOs: 29-46; of the DRD2 gene comprising at least one polymorphism of SEQ ID NOs: 47-51; of the DRD4 gene comprising at least one polymorphism of SEQ ID NOs: 52-54; of the FKBP5 gene comprising at least one polymorphism of SEQ ID NOs: 55-64; of the GCR gene comprising at least one polymorphism of SEQ ID NOs: 65-71; of the HTR2A gene comprising at least one polymorphism of SEQ ID NOs: 72-76; of the HTR2C gene comprising the polymorphism of SEQ ID NO: 77; of the NPY gene comprising at least one polymorphism of SEQ ID NOs: 78-79; of the NT-3 gene comprising at least one polymorphism of SEQ ID NOs: 80-83; of the NTRK2 gene comprising at least one polymorphism of SEQ ID NOs: 84-93; of the OPRM1 gene comprising at least one polymorphism of SEQ ID NOs: 94-96; of the SLC6A2 gene comprising at least one polymorphism of SEQ ID NOs: 97-98; of the SLC6A3 gene comprising at least one polymorphism of SEQ ID NOs: 99-110 or of the SLC6A4 gene comprising at least one polymorphism of SEQ ID NOs: 111-118.

The present invention provides a nucleic acid sequence of the ABCB1 gene comprising at least one polymorphism of SEQ ID NOs: 1-15; of the ADCYAP1R1 gene comprising the polymorphism of SEQ ID NO: 16; of the ADRA2A gene comprising at least one polymorphism of SEQ ID NOs: 17-18; of the BDNF gene comprising at least one polymorphism of SEQ ID NOs: 19-20; of the COMT gene comprising at least one polymorphism of SEQ ID NOs: 21-23; of the CRHBP gene comprising the polymorphism of SEQ ID NO: 24; of the CRHR1 gene comprising at least one polymorphism of SEQ ID NOs: 25-28; of the DBI gene comprising at least one polymorphism of SEQ ID NOs: 29-46; of the DRD2 gene comprising at least one polymorphism of SEQ ID NOs: 47-51; of the DRD4 gene comprising at least one polymorphism of SEQ ID NOs: 52-54; of the FKBP5 gene comprising at least one polymorphism of SEQ ID NOs: 55-64; of the GCR gene comprising at least one polymorphism of SEQ ID NOs: 65-71; of the HTR2A gene comprising at least one polymorphism of SEQ ID NOs: 72-76; of the HTR2C gene comprising the polymorphism of SEQ ID NO: 77; of the NPY gene comprising at least one polymorphism of SEQ ID NOs: 78-79; of the NT-3 gene comprising at least one polymorphism of SEQ ID NOs: 80-83; of the NTRK2 gene comprising at least one polymorphism of SEQ ID NOs: 84-93; of the OPRM1 gene comprising at least one polymorphism of SEQ ID NOs: 94-96; of the SLC6A2 gene comprising at least one polymorphism of SEQ ID NOs: 97-98; of the SLC6A3 gene comprising at least one polymorphism of SEQ ID NOs: 99-110 or of the SLC6A4 gene comprising at least one polymorphism of SEQ ID NOs: 111-118.

The present invention also provides methods for determining or predicting an anti-depressant or psychiatric drug response in a patient in need thereof by obtaining a biological sample from said patient; assaying the biological sample for the presence of at least one (e.g., at least 1, 2, 3, 4, or more) polymorphism in at least one (e.g., at least 1, 2, 3, 4, or more) pharmacogene in said sample, wherein the presence of at least one (e.g., at least 1, 2, 3, 4, or more) polymorphism indicates a modified response to the anti-depressant therapy. The at least one pharmacogene is selected from the pharmacogenes in Table 2. The at least one polymorphism in at least one pharmacogene is selected from SEQ ID NOs: 1-118.

In addition, the invention provides a method for interrogating thousands of aggregated whole human genome sequences, the method including (a) using a targeted analysis of one or more selected pharmacogenes and (b) determining polymorphic sequences that may associate with a drug response. The method can be executed on an inexpensive, energy-efficient, and heterogeneous graphics processing unit (GPU)-cluster based workstation.

In one embodiment, the method comprises the steps of (a) aggregating and performing a concordance check on populations of completed whole genome DNA sequences; (b) scanning assembled whole human genomes for target enrichment of one or more selected pharmacogenes, wherein the scanning is performed by using genome browser coordinates for the one or more selected pharmacogenes based on user input; (c) applying a multi-genome variant analysis algorithm to identify gene variants in said one or more pharmacogenes; (d) optionally, applying an algorithm to identify a potentially deleterious mutation that could impact a drug response; and (e) detecting a single nucleotide polymorphism (SNP), a multi-nucleotide polymorphism (MNP) or both SNP and MNP, but not other structural variants, and applying a statistical error-checking method to validate the SNP, MNP, or both SNP and MNP having allele frequencies of 0.1% to 99%.

In one embodiment, the pharmacogenes include the ABCB1 gene, the ADCYAP1R1 gene, the ADRA2A gene, the BDNF gene, the COMT gene, the CRHBP gene, the CRHR1 gene, the DBI gene, the DRD2 gene, the DRD4 gene, the FKBP5 gene, the GCR gene, the HTR2A gene, the HTR2C gene, the NPY gene, the NT3 gene, the NTRK2 gene, the OPRM1 gene, the SLC6A2 gene, the SLC6A3 gene, and the SLCA4 gene.

In an embodiment of the methods of the invention, the SNP, MNP, or both SNP and MNP is selected from one or more of the polymorphisms identified in SEQ ID NOs: 1-15 (gene: ABCB1), 16 (ADCYAPIR1), 17-18 (ADRA2A), 19-20 (BDNF), 21-23 (COMT), 24 (CRHBP), 25-28 (CRHR1), 29-46 (DBI), 47-51 (DRD2), 52-54 (DRD4), 55-64 (FKBP5), 65-71 (GCR), 72-76 (HTR2A), 77 (HTR2C), 78-79 (NPY), 80-83 (NT3), 84-93 (NTRK2), 94-96 (OPRM1), 97-98 (SLC6A2), 99-110 (SLC6A3), and 111-118 (SLC6A4).

The invention also features a method for determining the likelihood of an adverse or modified response to an anti-depressant or psychiatric drug in a patient in need thereof. The method includes obtaining a biological sample from said patient and assaying the biological sample for the presence at least one polymorphism in one or more pharmacogenes selected from those polymorphisms identified in SEQ ID NOs: 1-118. The presence of at least one polymorphism indicates that an adverse or modified response to the anti-depressant or psychiatric drug is likely.

Exemplary anti-depressant or psychiatric drugs include but are not limited to clozapine, fluvoxamine, escitalopram, paroxetine, amitriptyline, venlafaxine, citalopram, risperidone, nortriptyline, fluoxetine, olanzapine, tricyclic antidepressants, selective serotonin reuptake inhibitors, mitrtazapine, oxymetazoline, clonidine, epinephrine, norepinephrine, phenylephrine, dopamine, p-synephrine, p-tyramine, serotonin, p-octopamine, yohimbine, phentolamine, mianserine, chlorpromazine, spiperone, prazosin, propranolol, alprenolol, and pindolol.

The invention includes an isolated nucleic acid consisting of any one of the sequences identified by SEQ ID NOs: 1-118. In some aspects, the nucleic acid is a cDNA. The invention also includes a vector comprising an isolated nucleic acid consisting of any one of the sequences identified by SEQ ID NOs: 1-118. In addition, the invention includes a cell comprising an isolated nucleic acid consisting of any one of the sequences identified by SEQ ID NOs: 1-118.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims.

### BRIEF DESCRIPTION OF THE DRAWINGS

FIG. 1 is a schematic illustration of a novel polymorphism detection workflow of the present invention.

FIG. 2 is a graphical representation of the Bioinformatics workflow of the present invention.

FIG. 3 shows the method for aggregation and concordance checking of whole human genome sequences from multiple vendors.

FIG. 4 shows the target-enrichment module that allows the user to sequentially enter selected pharmacogenes of interest and that scans complete whole human genomes for pharmacogene sequences.

FIG. 5 shows the logic flow of the human genome population variant analysis algorithm.

FIG. 6 shows how the sliding window algorithm exploits texture memory in the CUDA architecture.

FIG. 7A lists data storage and transfer rate requirements for interactions between the different parts of the invention, based on current analysis of 17,131 whole human genomes.

FIG. 7B lists additional data storage and transfer rate requirements for interactions between the different parts of the invention, based on current analysis of 17,131 whole human genomes.

FIG. 8 shows the composition of 17,131 whole genomes used for testing the invention and the associated demographic data.

FIG. 9 lists the selected pharmacogenes that may impact drug response in psychiatry.

FIG. 10 shows a common use of the sliding algorithm in bioinformatics and other applications.

FIG. 11 shows a comparison of the alignment and variant analysis programs.

FIG. 12 shows the Pigeon hole filter associated with the sliding window algorithm.

FIG. 13 shows the accurate alignment computation in the GPU for a 1×2 mesh.

FIG. 14 shows that the HUGEPOPS algorithm performs both horizontal and vertical sliding window algorithms in parallel.

FIG. 15 is a schematic depicting a number of identified SLC6A2 SNPs.

FIG. 16 shows the comparison of the 5-HTTLPR MNPs in the SLC6A4 gene across racial subpopulations.

### DETAILED DESCRIPTION OF THE INVENTION

The present invention provides methods for interrogating thousands of aggregated whole human genome sequences, using targeted analysis of selected pharmacogenes, determining polymorphic sequences that may associate with drug response, executed on an inexpensive, energy-efficient, heterogeneous GPU-cluster based workstation.

The methods include aggregating populations of completed whole genome DNA sequences, and performing a concordance check. The methods include scanning assembled whole human genomes for target enrichment of selected pharmacogenes, using genome browser coordinates for selected pharmacogenes based on user input. The methods include applying a multi-genome variant analysis algorithm to identify gene variants in said pharmacogenes, consisting of detection of novel single nucleotide polymorphisms (SNPs) and multi-nucleotide polymorphisms (MNPs), but not other structural variants, and applying statistical error-checking methods to validate SNPs and MNPs with allele frequencies of 0.1% to 99%.

The targeted, selected pharmacogenes contain previously undetected nucleotide polymorphisms, including SNPs and MNPs. For example the ABCB1 gene contains 15 single nucleotide polymorphisms. The ADCYAP1R1 gene contains 5 single nucleotide polymorphisms and 1 multi-nucleotide polymorphism. The ADRA2A gene contains 2 single nucleotide polymorphisms and 1 multi-nucleotide polymorphism. The BDNF gene contains 2 single nucleotide polymorphisms. The COMT gene contains 3 single nucleotide polymorphisms. The CRHBP gene contains 5 single nucleotide polymorphisms. The CRHR1 gene contains 5 single nucleotide polymorphisms. The DBI gene contains 18 single nucleotide polymorphisms and 2 multi-nucleotide polymorphisms. The DRD2 gene contains 5 single nucleotide polymorphisms. The DRD4 gene contains 4 single nucleotide polymorphisms. The FKBP5 gene contains 10 single nucleotide polymorphisms. The GCR(NR3C1) gene contains 7 single nucleotide polymorphisms. The HTR2A gene contains 8 single nucleotide polymorphisms. The HTR2C gene contains 1 single nucleotide polymorphism and 2 multi-nucleotide polymorphisms. The NPY gene contains 2 single nucleotide polymorphisms. The NT3 gene contains 7 single nucleotide polymorphisms. The NTRK2 gene contains 10 single nucleotide polymorphisms. The OPRM1 gene contains 3 single nucleotide polymorphisms and 1 multi-nucleotide polymorphism. The SLC6A2 gene contains 2 single nucleotide polymorphisms and 2 multi-nucleotide polymorphisms. The SLC6A3 gene contains 12 single nucleotide polymorphisms. The SLC6A4 gene contains 10 single nucleotide polymorphisms and 1 multi-nucleotide polymorphism. The pharmacogene single nucleotide polymorphisms and multi-nucleotide polymorphisms identified by the methods of the invention are reported in a database.

The present invention provides a nucleic acid sequence comprising at least 5, at least 10, at least 15 or at least 50 continuous nucleotides of the ABCB1 gene comprising at least one (e.g., at least 1, 2, 3, 4, or more) polymorphism of SEQ ID NOs: 1-15; of the ADCYAP1R1 gene comprising the polymorphism of SEQ ID NO: 16; of the ADRA2A gene comprising at least one (e.g., at least 1, 2, 3, 4, or more) polymorphism of SEQ ID NOs: 17-18; of the BDNF gene comprising at least one (e.g., at least 1, 2, 3, 4, or more) polymorphism of SEQ ID NOs: 19-20; of the COMT gene comprising at least one polymorphism (e.g., at least 1, 2, 3, 4, or more) of SEQ ID NOs: 21-23; of the CRHBP gene comprising the polymorphism of SEQ ID NO: 24; of the CRHR1 gene comprising at least one (e.g., at least 1, 2, 3, 4, or more) polymorphism of SEQ ID NOs: 25-28; of the DBI gene comprising at least one (e.g., at least 1, 2, 3, 4, or more) polymorphism of SEQ ID NOs: 29-46; of the DRD2 gene comprising at least one (e.g., at least 1, 2, 3, 4, or more) polymorphism of SEQ ID NOs: 47-51; of the DRD4 gene comprising at least one (e.g., at least 1, 2, 3, 4, or more) polymorphism of SEQ ID NOs: 52-54; of the FKBP5 gene comprising at least one (e.g., at least 1, 2, 3, 4, or more) polymorphism of SEQ ID NOs: 55-64; of the GCR gene comprising at least one (e.g., at least 1, 2, 3, 4, or more) polymorphism of SEQ ID NOs: 65-71; of the HTR2A gene comprising at least one (e.g., at least 1, 2, 3, 4, or more) polymorphism of SEQ ID NOs: 72-76; of the HTR2C gene comprising the polymorphism of SEQ ID NO: 77; of the NPY gene comprising at least one (e.g., at least 1, 2, 3, 4, or more) polymorphism of SEQ ID NOs: 78-79; of the NT-3 gene comprising at least one (e.g., at least 1, 2, 3, 4, or more) polymorphism of SEQ ID NOs: 80-83; of the NTRK2 gene comprising at least one (e.g., at least 1, 2, 3, 4, or more) polymorphism of SEQ ID NOs: 84-93; of the OPRM1 gene comprising at least one (e.g., at least 1, 2, 3, 4, or more) polymorphism of SEQ ID NOs: 94-96; of the SLC6A2 gene comprising at least one (e.g., at least 1, 2, 3, 4, or more) polymorphism of SEQ ID NOs: 97-98; of the SLC6A3 gene comprising at least one (e.g., at least 1, 2, 3, 4, or more) polymorphism of SEQ ID NOs: 99-110 or of the SLC6A4 gene comprising at least one (e.g., at least 1, 2, 3, 4, or more) polymorphism of SEQ ID NOs: 111-118.

The present invention provides a nucleic acid sequence of the ABCB1 gene comprising at least one polymorphism of SEQ ID NOs: 1-15; of the ADCYAP1R1 gene comprising the polymorphism of SEQ ID NO: 16; of the ADRA2A gene comprising at least one polymorphism of SEQ ID NOs: 17-18; of the BDNF gene comprising at least one polymorphism of SEQ ID NOs: 19-20; of the COMT gene comprising at least one polymorphism of SEQ ID NOs: 21-23; of the CRHBP gene comprising the polymorphism of SEQ ID NO: 24; of the CRHR1 gene comprising at least one polymorphism of SEQ ID NOs: 25-28; of the DBI gene comprising at least one polymorphism of SEQ ID NOs: 29-46; of the DRD2 gene comprising at least one polymorphism of SEQ ID NOs: 47-51; of the DRD4 gene comprising at least one polymorphism of SEQ ID NOs: 52-54; of the FKBP5 gene comprising at least one polymorphism of SEQ ID NOs: 55-64; of the GCR gene comprising at least one polymorphism of SEQ ID NOs: 65-71; of the HTR2A gene comprising at least one polymorphism of SEQ ID NOs: 72-76; of the HTR2C gene comprising the polymorphism of SEQ ID NO: 77; of the NPY gene comprising at least one polymorphism of SEQ ID NOs: 78-79; of the NT-3 gene comprising at least one polymorphism of SEQ ID NOs: 80-83; of the NTRK2 gene comprising at least one polymorphism of SEQ ID NOs: 84-93; of the OPRM1 gene comprising at least one polymorphism of SEQ ID NOs: 94-96; of the SLC6A2 gene comprising at least one polymorphism of SEQ ID NOs: 97-98; of the SLC6A3 gene comprising at least one polymorphism of SEQ ID NOs: 99-110 or of the SLC6A4 gene comprising at least one polymorphism of SEQ ID NOs: 111-118.

The present invention also provides methods for determining an anti-depressant or psychiatric drug response in a patient in need thereof by obtaining a biological sample from said patient; assaying the biological sample for the presence at least one (e.g., at least 1, 2, 3, 4, or more) polymorphism in at least one (e.g., at least 1, 2, 3, 4, or more) pharmacogene in said sample, wherein the presence of at least one polymorphism indicates a modified response to the anti-depressant therapy. The at least one pharmacogene is selected from the pharmacogenes in Table 2. The at least one polymorphism in at least one pharmacogene is selected from SEQ ID NOs: 1-118.

The definition of pharmacogenomics by the U.S. FDA is the study of variations of deoxyribonucleic acid (DNA) and ribonucleic acid (RNA) characteristics as related to drug response. Pharmacogenetics relies on the application of common single nucleotide polymorphisms (SNPs) or combinations of SNPs to detect variations between individuals, or subpopulations of patients, that affect drug response or adverse drug events based on genotype. The customary focus used in pharmacogenetics has been on genes that encode pharmacokinetic proteins, such as the family of cytochrome P450 metabolic enzymes.

Pharmacogenomics uses data from whole human genomes or exomes, encompassing the entirety of SNPs and MNPs, haplotype markers, or alterations in gene expression or inactivation that may be correlated with pharmacological function and therapeutic response to a drug. Pharmacogenomics uses genetic sequence and genomics information in patient management to enable therapy decisions. In some cases, the pattern or profile of the change rather than the individual biomarker is relevant to diagnosis. In pharmacogenomics, researchers are able to look at variations in all the genes in a group of individuals simultaneously to determine the basis for variations in drug response. In pharmacogenomics, a gene is a locatable region of genomic sequence, corresponding to a unit of inheritance, which is associated with regulatory regions, transcribed regions, and/or other functional sequence regions.

With the knowledge that certain genetic changes result in alterations in patient responses to drugs, the hope is that clinicians will be better able to make decisions about treatments for their patients. An individual patient has an inherited ability to metabolize, eliminate, and respond to specific drugs. Correlation of polymorphisms with pharmacogenomic traits identifies those polymorphisms that impact drug toxicity and treatment efficacy. This information can be used by doctors to determine what course of medicine is best for a particular patient and by pharmaceutical companies to develop new drugs that target a particular disease or particular individuals within the population, while decreasing the likelihood of adverse effects. Drugs can be targeted to groups of individuals who carry a specific allele or group of alleles. For example, individuals who carry allele A1 at polymorphism A may respond best to medication X while individuals who carry allele A2 at polymorphism A respond best to medication Y. A trait may be the result of a SNP, MNP, an interplay of several genes or gene polymorphisms, or through gene by environment interactions.

In addition, some drugs that are highly effective for a large percentage of the population prove dangerous or even lethal for a very small percentage of the population. These drugs typically are not available to anyone. Pharmacogenomics can be used to correlate a specific genotype with an adverse drug response. If pharmaceutical companies and physicians can accurately identify those patients who would suffer adverse responses to a particular drug, the drug can be made available on a limited basis to those who would benefit from the drug.

In the clinical setting, pharmacogenomics may enable clinicians to select the appropriate pharmaceutical agents, and the appropriate dosage of these agents, for each individual patient. That is, pharmacogenomics can identify those patients with the right genetic makeup to respond to a given therapy, and also can identify those patients with genetic variations in the genes that control the metabolism of pharmaceutical compounds, so that the proper dosage can be administered. A pharmacogene is any gene involved in the response to a drug, and includes both pharmacodynamics genes (those that are associated with the effects of a drug on an individual) and pharmacokinetic genes (genes involved in the metabolism of a drug).

Although both SNP-based genotyping and whole genomic profiling provide increasing degrees of accuracy for guiding drug prescribing for the individual patient, data collected from pooled genomic sequences may provide even more power for such tests, especially when combined with targeted resquencing.

Targeted re-sequencing is a variation of re-sequencing where only a small subset of the genome is sequenced, such as the exome, a promoter (e.g., 5′-HTTLPR of SLC6A4), a particular chromosome, a set of genes, or a region of interest. By focusing all of the sequencing on a small region of the genome, it is possible to detect low levels of variation that might have otherwise been missed. Some researchers have started to use targeted re-sequencing for genome-wide association studies (GWAS) instead of arrays as it is better suited for measuring rare alleles. A subset of the genome is typically targeted in one of two main ways, either by amplifying the genes or region of interest with long range PCR, or by capturing the region of interest by hybridizing with complementary oligonucleotides.

In long range PCR, primers are designed against regions of interest, and the amplified products are purified and used as input for library preparation. Multiplexing the PCR reactions can improve the workflow and reduce costs. This method has the advantage of being relatively simple with no need for specialized equipment. However, it can be very laborious. Also, not all regions are easily amplified, and the region that can be amplified in a single reaction is fairly limited.

For the sequence capture (or target enrichment) method, there are two main subtypes. In the first subtype, capture is based on microarrays used for hybridization of targeted regions. A sequencing library is generated and then hybridized to the capture array. The portion of the library that was captured is then eluted off the array and sequenced. The second and more common method, solution-based capture, uses capture oligos (or baits), which are hybridized to the target DNA in solution. Those capture oligos that have bound to the complementary target DNA are then collected and purified using a magnetic bead-based system or other selection system. The target DNA is then eluted off the beads and sequenced. The array-based method is often used when the target design will only be used across a small number of samples (up to 20 or so) as it is easier to make small batches. The solution-based method scales more easily and is generally cheaper when used across a larger number of samples. Research shows that it outperforms the array-based method. Compared to the long range PCR method, both capture methods have the advantage of working with highly complex targets. They are currently less expensive than long range PCR, and costs are being driven down as more companies bring target enrichment solutions to the market.

Approaches that combine targeted loci known to be involved with drug response, with populations of pooled genome sequences, provide the optimal approach for identification of specific individual polymorphisms that are of most relevance to that individual's response to a drug. This is because it provides the most discrimination of that individual's pharmacogene variants, such as SNPs and MNPs, against a background of a much larger sample, locating the proverbial “needle in the haystack” that provides the best fit for that specific individual.

In the methods of the invention, targeted regions of interest (ROI), such as selected pharmacogenes, are chosen for sequencing across the mixed population library based upon collective insights into the biology of the drug response. Specific primers are designed to extract ROI from the population library by inverse PCR. Library circularization and inverse PCR allow the DNA bar-code to be retained during extraction. The resultant PCR reactions yield directly sequencable amplicons containing target regions from the individuals within the population library. Each PCR reaction is carried out separately, which allows primer design to be ‘singleplex’. This avoids problems associated with alternative multiplex extraction methods, and thus yields high physical coverage across targets. This approach itself avoids the need to sequence the entire genome; only the targeted ROI needs to be sequenced. Once extracted, all amplicons are pooled prior to sequencing using an appropriate next generation sequencing platform.

The resulting sequencing data are assembled for each amplicon, and sorted on a per individual basis by reading the unique DNA bar-code. Each individual within the population library is identified as homozygous or heterozygous for any variants identified. Such variants may be rare single nucleotide polymorphisms (SNPs) or small insertions or deletions.

This approach works well if a large number of biological samples containing both the genomic DNA from a large pool of human genomes are available for extraction and sequencing, along with DNA extracted from a given individual that will be prescribed a drug based on how their polymorphisms differ from the larger pool of sequences.

However, the emergence of thousands to millions of whole human genome sequences mitigates the need to collect both pooled population samples as a background for precision resolution of any one individual's pattern of pharmacogene polymorphisms that are determinative for personalization of drug efficacy and toxicity. Thus, by obtaining completed, whole genome sequences for analysis, and performing concordance checking, it is possible to determine stringent alignment between thousands of sequences when integrated into the same format. When using a targeting system as described herein, the concordance between pharmacogenes from these experiments has ranged from 99.4-99.8% versus 98.92% across the aligned sequences generated from three different sequencing platforms.

This invention addresses the next era of bioinformatics requirements—the need to run queries against large populations of human genome sequences, ChiPseq, RNAseq, and related aggregated data. Determining relationships between populations of whole genome sequences represents a first step in almost all studies that hinge on patterns of genetic variation. The most widely used algorithms in this emerging domain employ similarity/distance measures that can be constructed using genetic data, and are used in clustering algorithms to identify distinct ancestry profiles. An alternative approach is to examine the Principal Components, which is typically done two components at a time. For example, visualization using a heatmap of the ordered matrix of clusters shows the similarity between each one and may be more informative since it allows variation to be assessed simultaneously at multiple different levels. Although clustering the sample into ‘populations’ with discrete ancestry profiles also represents a useful starting point in approaches that seek to infer the historical processes that have led to differentiation between members of the sample, whether on short or long timescales, its assumptions are questionable. Unlike studies of historical ancestors of many millennia ago, when genome sequencing and analysis technology were not available but could have defined differences between racial/ethnic human genome populations with more accuracy, the examination of variation in studies such as the 1000 Genomes Project, which samples from presumably genetically more separated tribes or ethnic subpopulations, have demonstrated that “out-breeding” in these populations is much more prevalent than is assumed. Indeed, even statisticians have criticized the 1000 Genomes Project exon sequencing on a preponderance of false positive rare SNPs (Tintle et al, Genet Epidemiol. 2011; 35(Suppl 1): S56-S60 2011), which is equally explained by the presence of rare variants through mating with unrelated individuals.

One of the most exciting prospects of whole-genome polymorphism data is the increased power to characterize not only the recent adaptive history of natural populations, but also the prevalence of positive and negative natural selection. Negative selection reduces variation in the genome by eliminating some mutations, holding others to low frequency, and also causing the loss of variants linked to deleterious alleles (background selection). As a favorable mutation increases in frequency in a population, linked neutral variants will either become fixed along with it or be lost from the population. The size of the region of the genome affected by such a “selective sweep” is determined mainly by the strength of selection and the rate of recombination.

It has been argued that well mapped, aligned, calibrated reads, and assembled whole genomes cannot be relied on to accurately identify SNPs, MNPs, and other structural variants without application of statistical error correction to separate artifacts generated by next generation sequencing platforms from real genomic variation. Elaborate statistical methods have been applied to decrease the number of Type I false-positive errors and other machine artifacts. On the other hand, some have argued that every SNP found with genome-wide significance should be validated on another platform to verify that its significance is not an artifact of study design—the College of American Pathologists says that accurately matched genome sequences generated by 2 different sequencing machines determines accuracy.

In the past, when genome sequence assembly was a priority, many algorithms in bioinformatics have used just the GPU mainly to speed up just the fitness evaluation (usually the most time-expensive process). However, as the programming tools improve, newer computational approaches run the whole optimization algorithm on the GPU side, with diminished need of CPU interaction.

The present invention provides novel methods for the aggregation, concordance, and target enrichment of selected pharmacogenes based on user input, as well as multi-genome analysis and error-checking. The methods are scalable to tens of thousands of completed human genome sequence data. The invention further provides for analysis of the pooled DNA sequences, which may be specifically designed to interrogate the desired selected pharmacogenes for particular characteristics, such as, for example, the presence or absence of a polymorphism.

The present invention provides methods for identification of novel variants in pharmacodynamics genes that have been identified in the scientific literature as being associated with inter-patient differences in drug response to a psychotropic medication. The process includes target-enriched analysis of gene sequences and their flanking regions, including exons (protein-coding domains), introns (intervening sequences) and promoter sequences (transcriptional regulatory sequences) from a pool of 17,131 whole human genomes obtained from public sources. These whole genomes provide a sample of the residents of the United States identified as to age, race and gender, combined from data acquired from three different sequencing technologies. Imputation of critical genomic variants, including single nucleotide polymorphisms and other variants show that these novel variants have deleterious consequences for psychotropic drug response. This invention provides a foundation for optimizing the configuration of a whole genome-based pharmacogenomics test to guide drug therapy in psychiatry, using aggregated whole genomic profiling of individual patients, rather than single or combinations of single nucleotide polymorphism genotype-based pharmacogenetic tests.

This invention provides a method for analysis of thousands of whole human genome sequences to detect novel polymorphisms in selected pharmacogenes that have been associated with drug response in psychiatry. Disclosed are novel polymorphisms have been detected in genes that mediate psychotropic drug response. The whole genome, sequence-based analysis method described herein, is a more accurate, faster, less-expensive, and more efficient strategy to discover potentially deleterious gene mutations that may impact psychotropic drug response when compared to existing methods that rely on the use selected pharmacogenes based on published single nucleotide polymorphisms and multi-nucleotide polymorphisms drawn from existing published scientific and medical literature that have relied on genome-wide association studies (GWAS) that provide less accurate data. Combining novel polymorphisms discovered by this strategy with known variants that associate with inter-patient variability in drug response in psychiatry, delivers an aggregated molecular diagnostic test that provides a more powerful approach than previously available for directing medication therapy in psychiatry based on targeted genomic profiling within the context of a large pool of complete whole genome sequences.

The invention comprises five integrated and distinct parts: (1) Use of a desktop workstation for efficient, rapid and accurate collection of pooled human genome sequences, ranging from thousands to millions of said sequence data, featuring cloud storage and fast input/output and data transfer rates, (2) Aggregation and concordance checking of whole human genome sequences generated by more than 1 sequencing platform/technology, (3) Target enrichment of the pooled sequences en masse using genome browser coordinates selected by the user for choice of targeted sequences, followed by extraction of said sequences into an ordered and indexed matrix, (4) Application of a novel “climbing” algorithm analysis that interrogates every base in a ordered arrangement of the sequences, and separates using masking and alignment with 1 or more reference sequences, and classifying said SNP-containing and MNP-containing sequences into separate bins, and (5) Reporting to a database and outputting to a user interface.

(1) Use of a desktop workstation for efficient, rapid and accurate collection of aggregated human genome sequences, ranging from thousands to millions of said sequence data, featuring cloud storage and fast input/output and data transfer rates. Increases in supercomputing power achieved through parallelization using mutli-threaded GPUs, distributed cluster computing and Fast Programmable Gate Array (FPGA) technology has brought the ability to analyze thousands of whole human genome sequences to the desktop workstation, as demonstrated by this invention. In the present configuration, algorithms are designed to take advantage of multiple operations performed in a simultaneous manner, with simple arithmetic operations performed concurrently using distributed threads on the GPU, minimizing exchange of information between host CPU and device GPUs through the allocation of most functions to the CUDA cores. In the current configuration, power efficiency is achieved as well:

TABLE 1
Comparison of Analyzing 10,000 Whole
Genome Sequences on a Workstation

| | | | | Cost of | Cost of |
|---|---|---|---|---|---|
| | | | En- | Energy | Storage |
| Work- | | | ergy | per | per |
| station | Institution | Algorithm | Cost | Execution | Year |
| Invention | Home Office | HUGEPOPS | $0.13 | $1.20 | Onsite - |
| | | | kW-hr | | ~$1K |
| | | | | | Cloud - |
| | | | | | $10K |
| SeqNFind ™ | NHGRI* | GAMMA | $0.05 | $2.30 | Onsite - |
| | | | kW-hr | | $7M** |

*National Human Genome Research Institute - Figures from Laura Elnitski, Ph. D., Genome Technology Branch.
**Includes datacenter overhead. Based on data obtained Apr. 19, 2012.

(2) Aggregation and concordance checking of whole human genome sequences generated by more than 1 sequencing platform/technology. The present invention broadly relates to cost-effective, flexible and rapid methods for reducing nucleic acid sample complexity to enrich for target nucleic acids of interest and to facilitate further processing and analysis, based entirely on pooled genome sequence data, negating the need for sample collection, sample storage, and resquencing of samples. The captured target nucleic acid sequences, which are of a more defined, less complex genomic population are more amenable to detailed genetic analysis. Thus, the invention provides for methods for enrichment of target nucleic acid sequences against a background of a complex pooled population sample of sequences. Each data file must contain paired reads from a single library, a library split over many files, or a completed whole genome sequence such as would be delivered by Complete Genomics, Inc. as a tar file.

Accepted formats are fasta, fastq, fasta.gz, sam, bam, eland, gerald and tar. The algorithm is scalable. The files are all converted to AGP, the new NCBI standard, using the proprietary file conversion application called ‘MassConvert.’ This uses a modification of the public algorithm at the National Center for Biotechnology Information (NCBI) for AGP file conversion, that supports algorithm-based scaling to thousands to millions of genomes that are automatically aligned in any order in a neighbor-joining (NJ) mesh, consisting of an alignment algorithm that recognizes and assigns a start base, end base, strand and chromosome coordinate for every genome. This alignment algorithm is as follows: modification of the “Parallel progressive multiple sequence alignment on comparable meshes” It differs in that instead of being “global”, it is a hybrid algorithm that is “infitidunal”, that is, scalable to an ∞-1 number of sequences. The NJ takes a distance matrix between all the pairs of sequences and represents it as a connected matrix. NJ then finds the shortest distance pair of nodes and replaces it with a new node. This process is repeated until all the nodes are merged.

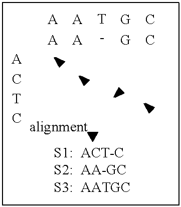

1. Initially, all the pair-wise distances are given in form of a matrix D of size m×m, where m is the number of input whole genome sequences.

2. Calculation is made to determine the average distance from node i to all the other nodes by ri=Σm1Dijm−2.

3. The pair of nodes with the shortest distance (i,j) is a pair that gives minimal value of Mij, where Mij=Dij−ri−rj.

4. A new node u is created for shortest pair (i,j), and the distances from u to i and j are: diu=Dij2+(ri−rj)2, and dj,u=dij−diu.

5. The distance matrix D is updated with the new node u to replace the shortest distance pair (i,j), and the distances from all the other nodes to u is calculated as Dvu=Div+djv−DU. These steps are repeated for m−1 iterations to reduce distance matrix D to one pair of nodes.

The difference as embodied in this algorithm of this invention is that when the progressive sequence alignment begins with a pre-aligned set of sequences, negating ‘progressive alignment’, only necessitating the pair-wise dynamic programming of two pre-aligned groups of sequences, avoiding the computationally expensive dynamic programming back-tracking on the r-mesh. This greatly increases the ‘speed-up’ when parallelized, as well as scalability of the algorithm to millions of long sequences.

(3) Target enrichment of the pooled sequences en masse using genome browser coordinates selected by the user for choice of targeted sequences. The method uses a modification of the MochiView software, which is written in Java, that transparently incorporates the Java DB database within the software. The database architecture is designed to scale well even with very large quantities of data (e.g, up to 5×10¹⁵ bytes of data without performance loss). (See, e.g., Homann and Johnson, MochiView: versatile software for genome browsing and DNA motif analysis BMC Biology 2010, 8:49 for all methods described herein). Promoter recognition is based on the method of Zeng et al. Briefings in Bioinformatics. Vol 10, No. 5. 498-508 (2009), incorporated herein by reference.

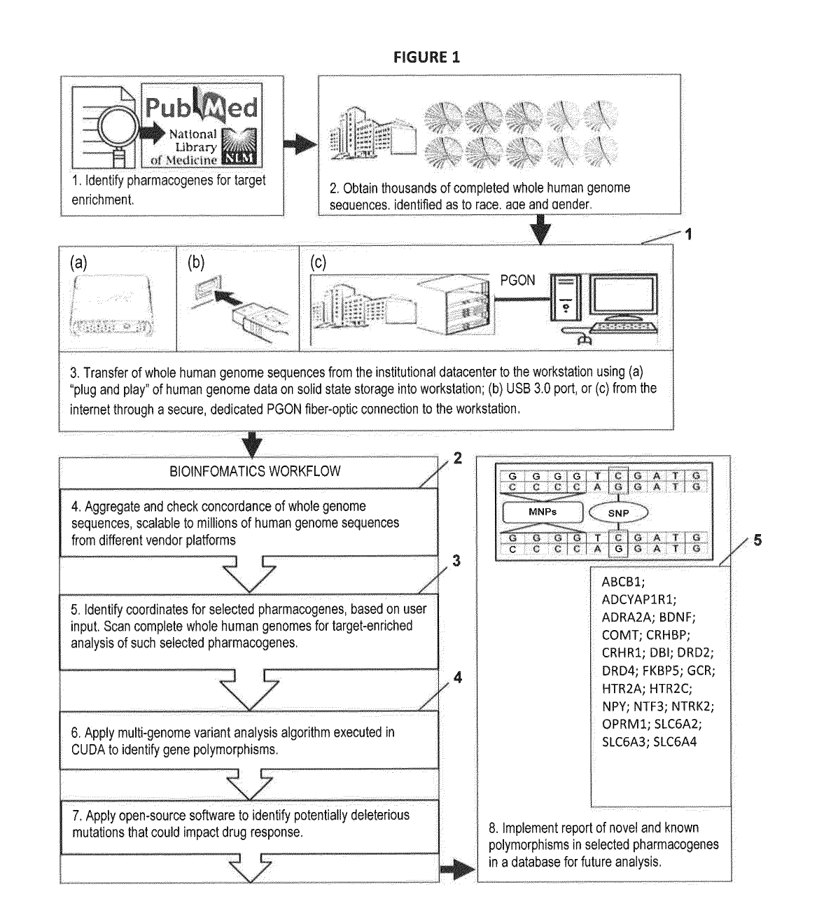

(4) Application of a sliding window algorithm analysis that interrogates every base in a ordered arrangement of the sequences, and separates using masking and alignment with 1 or more reference sequences, and classifying said SNP-containing and MNP-containing sequences into separate bins. The invention uses a novel application of the sliding window algorithm that has been used in genomic analyses, a general bioinformatics approach used in a number of genomic analyses. In this scenario, some property (e.g., sequence density) is computed for the portion of the genome within the bounds of a fixed window. As shown in FIG. 1, the window slides by a fixed amount across the genome, and the property is recomputed relative to the new window bounds. There are many different applications and variations of the sliding window approach, but they all follow this same general template. The sliding window technique is a widely used algorithmic primitive. For example, the sliding window approach has been used to improve the spatial resolution of predicted binding sites using ChIP-Seq data, DNA structural variations that are anomalies in a genome where portions of chromosomes have been added, deleted, or otherwise rearranged, and to analyze sequence polymorphisms.

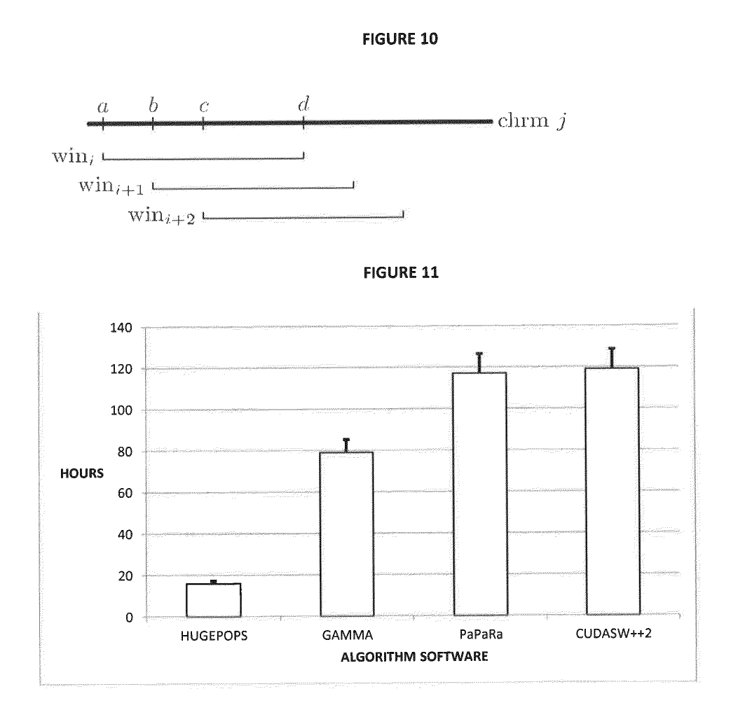

The sliding window algorithm has two main parameters, windows size and step size (i.e., the distance between successive windows). While window size is generally determined by experimental factors (e.g., sequence read length), step size is a tunable parameter and has a direct impact on accuracy and performance. Each window calculates a local statistic; as the step size increases, the gap between these statistics increases, which in turn decreases the resolution of any prediction (e.g., inflection points). As the step size decreases, more windows are required to analyze the genome, and the computational complexity becomes correspondingly larger. FIG. 10 shows a common use of the sliding algorithm in bioinformatics and other applications. In this case, the sliding window algorithm considers chromosome (chrom) j; where the window length is IdI-IaI, and the step size is IbI-IaI. Each window is offset from the previous window by the same step size.

Most recent attempts to parallelize high-throughput algorithms have been focused on algorithms that have large kernels that perform a large amount of computation per thread. In contrast, the sliding window algorithm has a small kernel and performs only a small amount of work per thread, making it a poor candidate for cluster-based parallelization, yet an ideal candidate for parallelization on Single Instruction Multiple Data (SIMD) architectures such as graphics processing units (GPUs) with highly multicore architectures such as NVIDIA's Compute Unified Device Architecture (CUDA) architecture for parallelizing the sliding window algorithm.

The Human Genome Population Polymorphism Sensor (HUGEPOPS) algorithm of the present invention provides the following superior, and unexpected, properties:

This is not a short read genome sequence assembly problem—these whole human genome sequences have been checked using redundant measures and can be easily ordered as to start and end points, so target coordinates of selected genes can be identified using a “loose” window to start the climbing algorithm;

Re-formulation of the sliding windows algorithm to run in both vertical and horizontal directions, comprising a anti-diagonal matrix, when comparing a query sequence, such as a specific selected pharmacogene, against a large pool of complete whole human genome sequences;

Parallelization of the algorithm to take advantage of texture cache memory in CUDA architecture to write 2D data, so that the sequence data does not have to access stored memory, which is very time consuming;

Perform optimized data compression within CUDA cores, using the Hoffman compression algorithm for JPEG compression, relieving any residual load on the CPU.

Match query lengths of the climbing algorithm to the registry values in CUDA.

In tests, only 0.25% of the data/algorithm require sequentical processing, which increases speed-up, according to Amdahl's Law. In the case of parallelization, Amdahl's law states that if P is the proportion of a program that can be made parallel (i.e., benefit from parallelization), and (1−P) is the proportion that cannot be parallelized (remains serial), then the maximum speedup that can be achieved by using N processors is:

$$S ( N ) = \frac{1}{( 1 - P ) + \frac{P}{N}} .$$

In the limit, as N tends to infinity, the maximum speedup tends to 1/(1−P). In practice, performance to price ratio falls rapidly as N is increased once there is even a small component of (1−P).

As an example, if P is 90%, then (1−P) is 10%, and the problem can be sped up by a maximum of a factor of 10, no matter how large the value of N used. For this reason, parallel computing is only useful for either small numbers of processors, or problems with very high values of P: so-called embarrassingly parallel problems. A great part of the craft of parallel programming consists of attempting to reduce the component (1−P) to the smallest possible value. P can be estimated by using the measured speedup SU on a specific number of processors NP using

$${P}_{estimated} = \frac{\frac{1}{SU} - 1}{\frac{1}{NP} - 1} .$$

P estimated in this way can then be used in Amdahl's law to predict speedup for a different number of processors.

Others have implemented local and global sequence alignment algorithms in the parallel CUDA environment, such as:

CUDASW++2: optimizing Smith-Waterman sequence database searches for CUDA-enabled graphics processing units;

GAMMA, multi-sequence variant analysis algorithm, developed by BGI.

PaPaRa: An alternative to the Smith-Waterman approach, distributing load to both GPUs and the CPU.

A comparison of these alignment and variant analysis programs is shown in FIG. 11, using a 32 base sequence query length against the dataset of assembled and pre-aligned genomes. FIG. 11 shows a mean±S.E.M of 6 runs. Statistical comparisons are not required to decide that HUGEPOPS has a speed-up of 4-fold against GAMMA, a variant detection algorithm that was developed for human genome research by BGI in association with NVIDIA Corporation. The units are not expressed in GCUPS (Giga Cell Units Per Second) because they are not suitable for such an application.

The workstation had ˜8Tflops, with the following characteristics: 8×C2075 Tesla Fermi GPUs with 6 GB memory, 12 MB cache comprising 2,888 CUDA cores; Dual Intel® Xeon X5690 CPU, hexa 3.46 GHz cores, 12 MB cache; 96 GB 1333 MHz ECC DDR3 main memory; 36 TB solid state storage and power consumption during execution of the HUGEPOPS algorithm: 25,600 watts over 16 hours.

The Human Genome Population Polymorphism Sensor (HUGEPOPS) comprises several components, taking advantage of the characteristics of the CUDA GPU that were designed for display of 3-dimensional graphics. In the broadest sense these include the following:

A. Re-formulation of a sliding window algorithm to include both horizontal and vertical windows (referred to as a “climbing” algorithm), creating a numerically redundant analysis that interrogates every base in a ordered arrangement of the sequences, and separates using masking and alignment with 1 or more reference sequences, and classifying said SNP-containing and MNP-containing sequences into separate bins.

B. Use of texture memory cache for running the parallelization algorithm, which is fine for 2D data analysis in this invention. The texture unit processes one group of four threads per cycle. Texture instruction sources are texture coordinates, and the outputs are filtered samples. Texture is a separate unit external to the SM connected via the SMC. The issuing SM thread can continue execution until a data dependency stall. Each texture unit has four texture address generators and eight filter units, for a peak Tesla Fermi rate of 1500 38.4 gigabilerps/s (a bilerp is a bilinear interpolation of four samples). Each unit supports full-speed 2:1 anisotropic filtering, as well as high-dynamic-range (HDR) 512-bit floating-point data format filtering. The texture unit is deeply pipelined. Although it contains a cache to capture filtering locality, it streams hits mixed with misses without stalling. Thus the HUGEPOPS algorithm can be executed without accessing global memory. It writes directly to the surface object, which would normally be used as a shader texture in 3D modeling and real-time simulation. The device memory automatically manages the cache, and provides boundary detection without computational deficit.

C. The HUGEPOPS algorithm defines any consecutive 12 base sequence from the pre-selected target pharmacogene sequence against aggregated and concordance-checked completed whole genome DNA sequences as a pattern. A pattern or read which contains any N will be ignored, since N signifies an unknown value read during the chemical process, in which case there is no point in matching that read. A mismatch is defined as unequal base pairs at the same offset in both the pattern and read. An insertion in a read (pattern) is defined as an extra base pair or more inserted at an offset only in the read (pattern), not the pattern (read). Likewise, a deletion in a read (pattern) is defined as a missing base pair at an offset only in the read (pattern), not the pattern (read). Note that an insertion in the pattern is equal to a deletion in the read and vice versa. Because the 17,131 whole genome sequences were completed, and checked before being sent to the National Institutes of Health, and we checked them again after receipt, and they were generated using different sequencing technologies and platforms, and as in the instantiation, targeting specific pharmacogenes that represent less than 0.5% of the reference genome, this greatly reduces the problem space in which HUGEPOPS has to operate. Thus, most of the assumptions that define a useful heuristic or other algorithm that is intended to assemble an entire whole genome sequence from short reads, as may be generated by next generation sequencing methods are ignored. This greatly reduces the complexity of the problem.

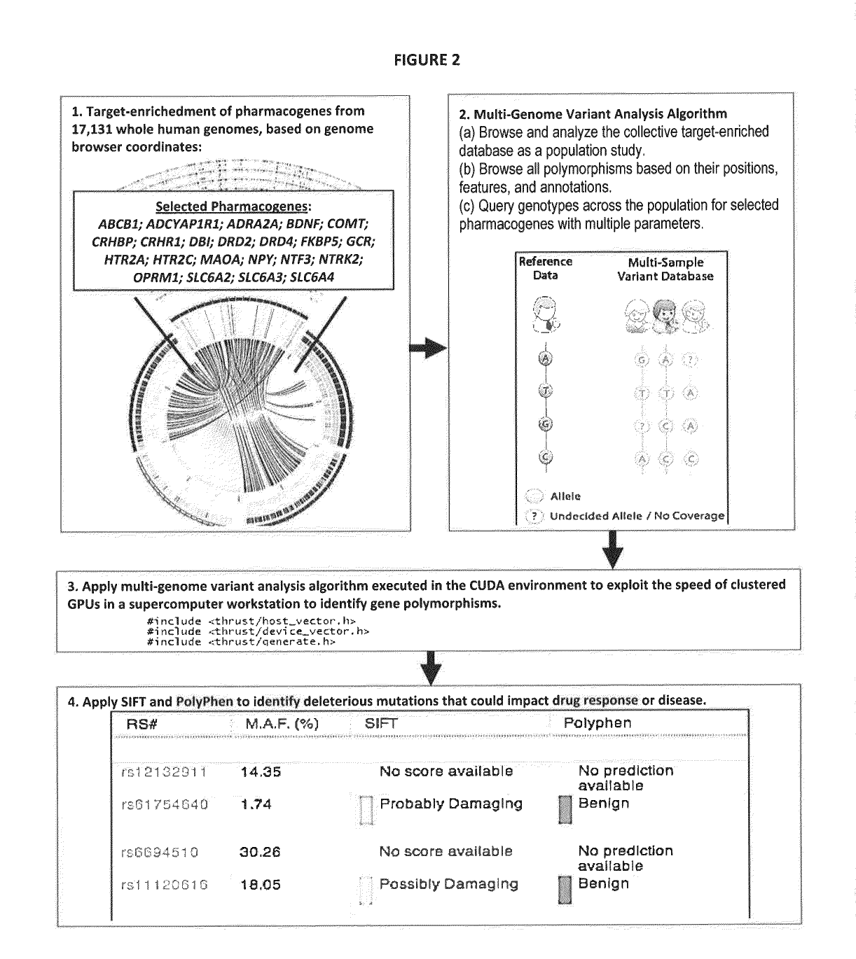

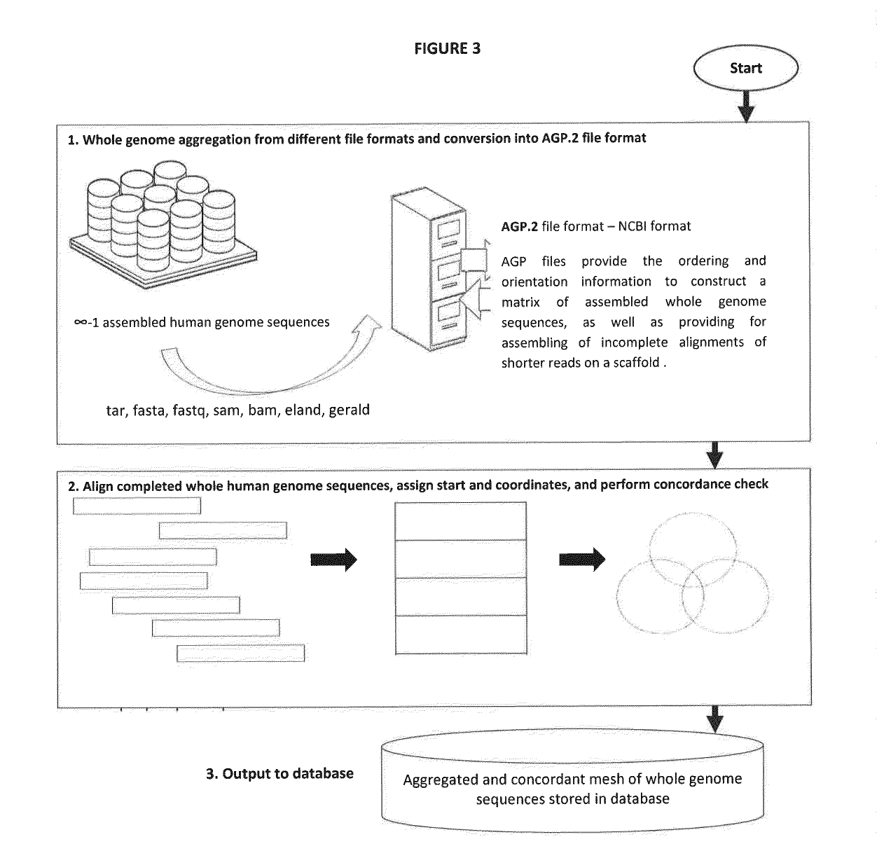

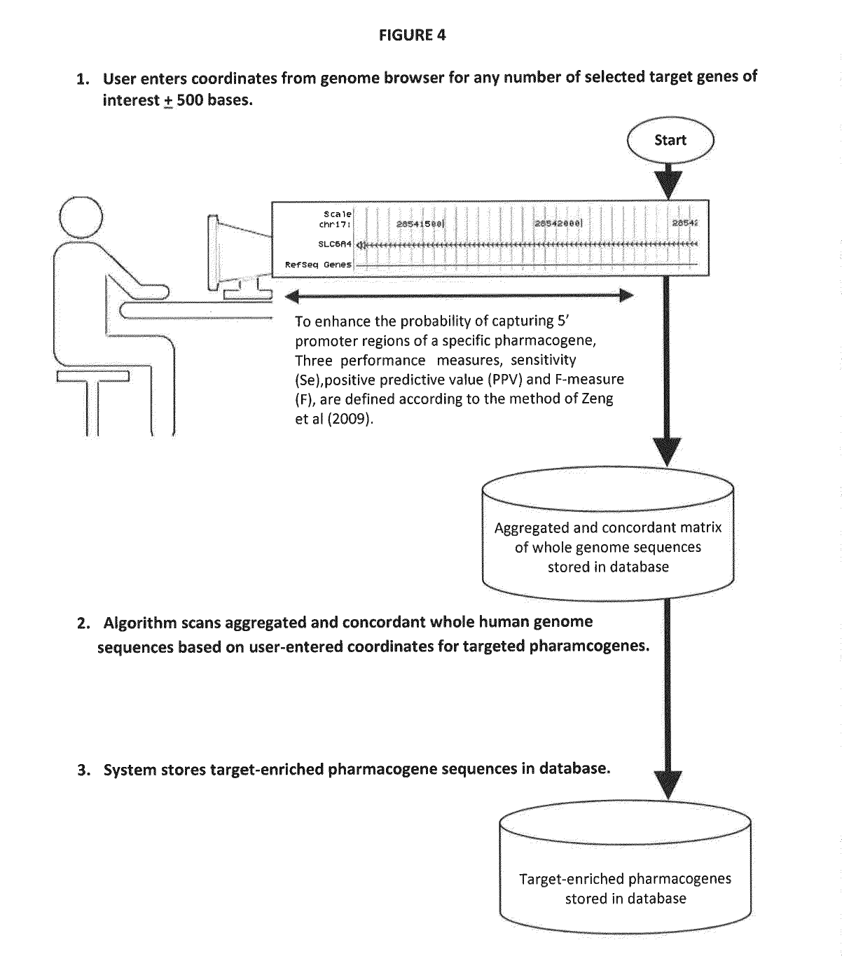

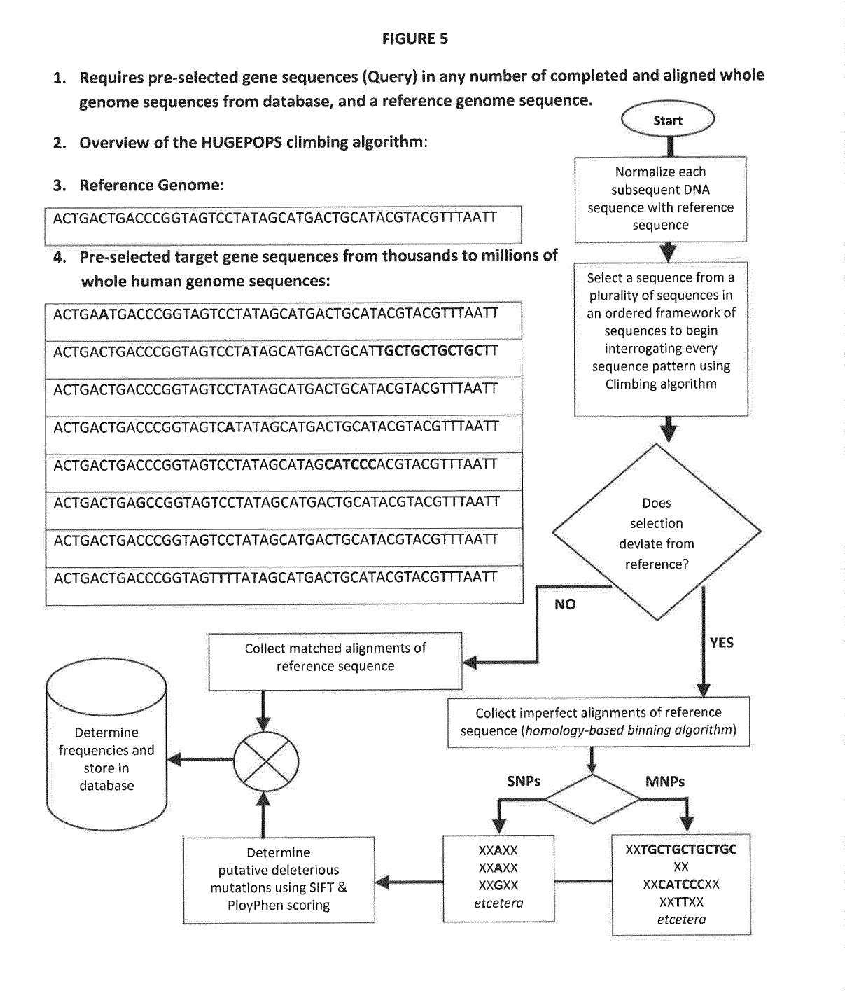

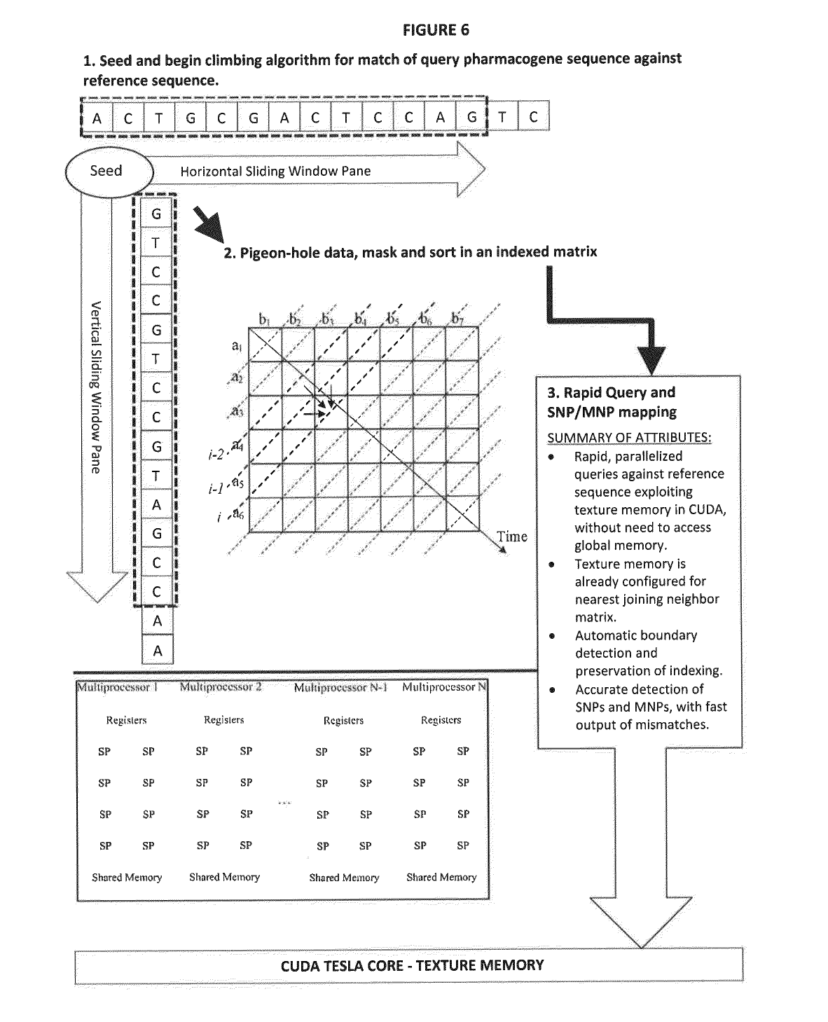

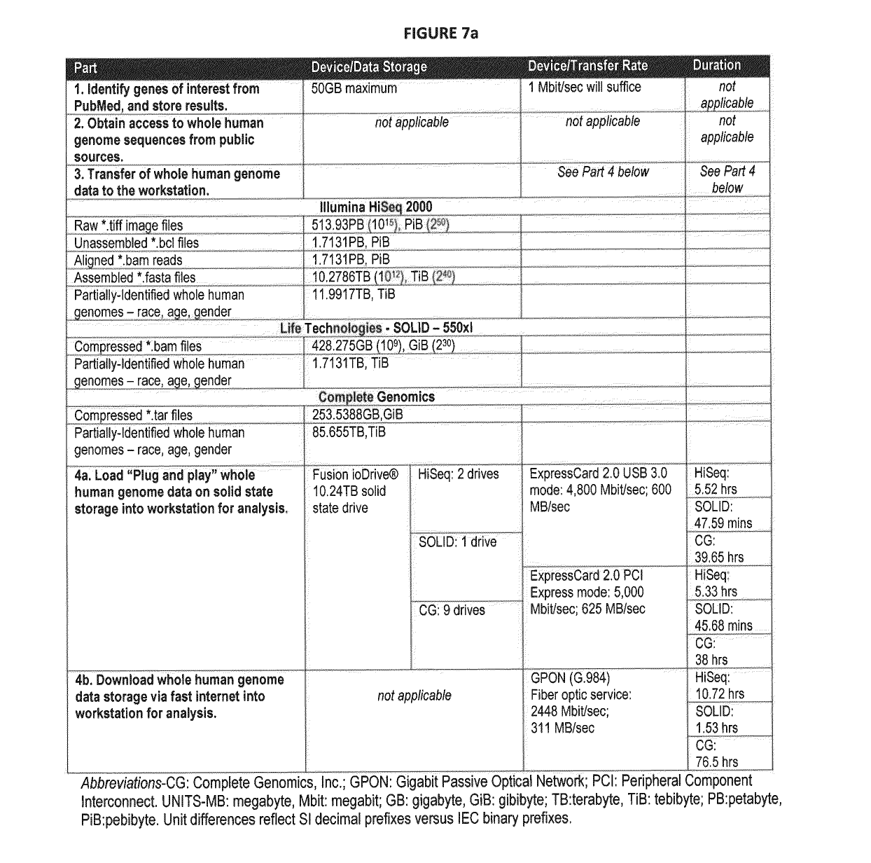

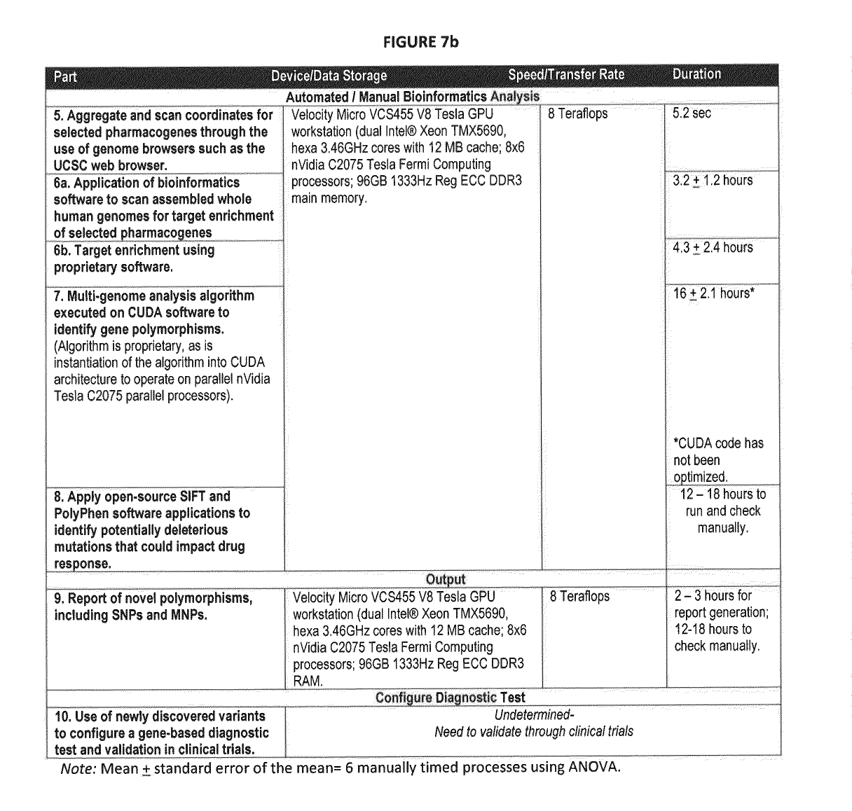

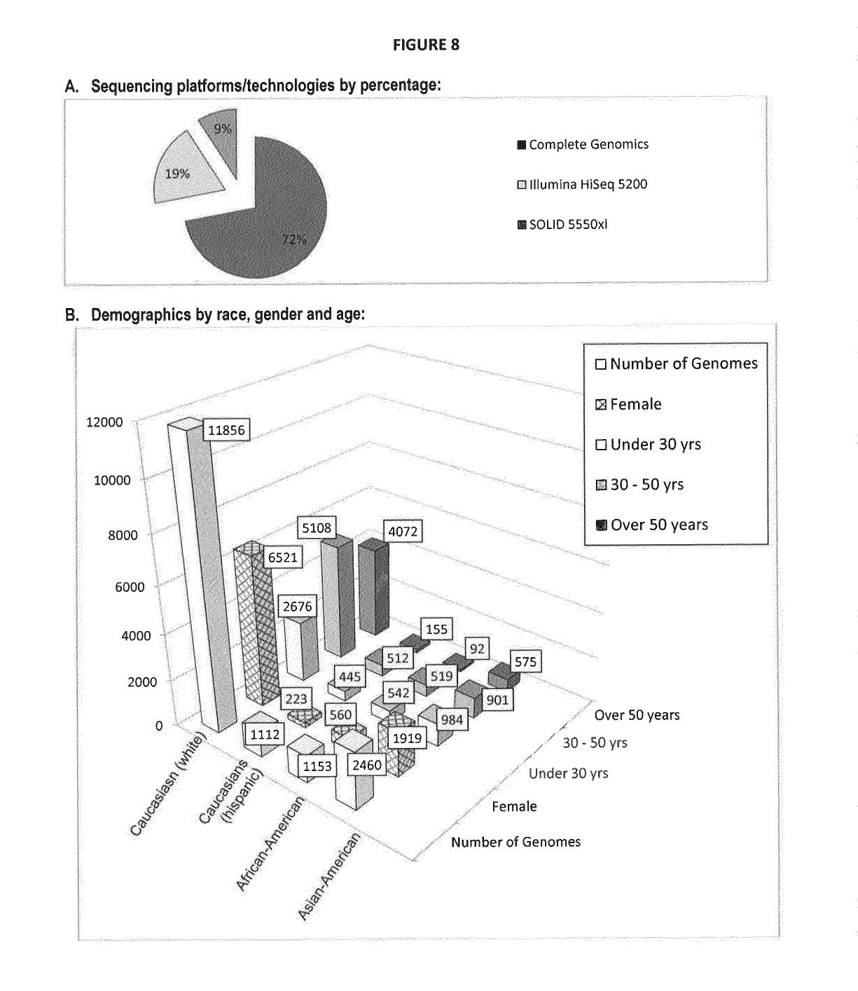

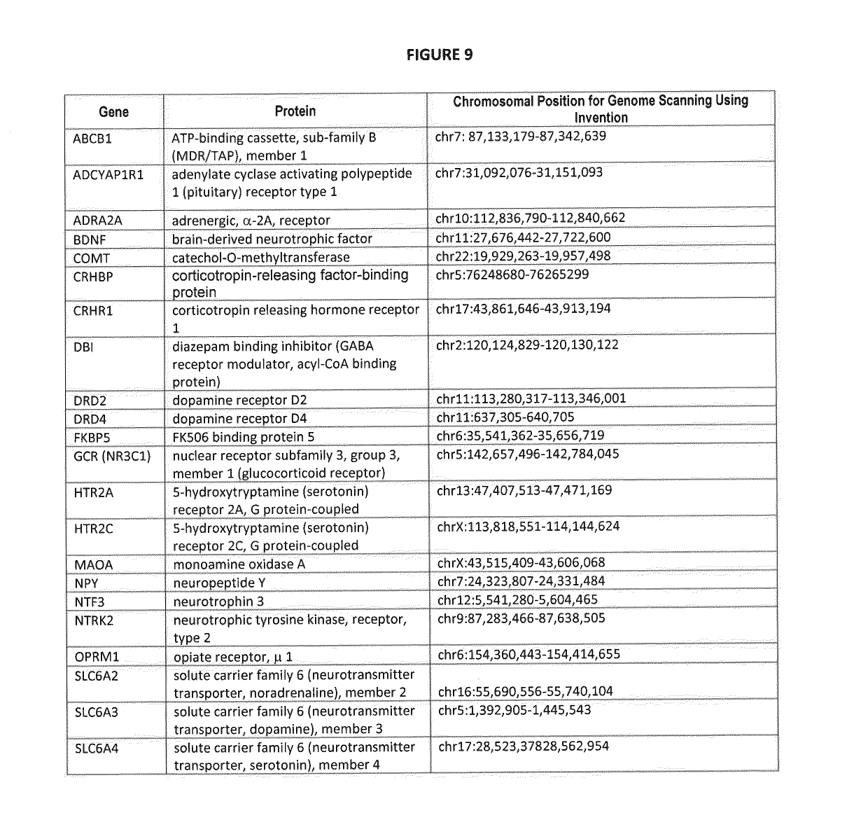

In the genome process step, a genome is split into patterns with length k (k=1/(d+1)) by using a sliding window-based scheme, called a “climbing algorithm”, and converted to numeric data type using 2-bits-per-base as shown in FIG. 2. However, unlike the typical scheme shown in FIG. 2, the size of both horizontal and vertical sliding window is equal to the length of pattern (See FIG. 3). Two data structures, seed and genome sliding window array, are utilized to record each seed and its position and sliding window position, respectively. The seed and sliding window array are stored in texture memory of the GPU. The algorithm performs highly parallelized exact query matching on the GPU. Each query sequence is matched against the reference sequence in time proportional to its length by navigating the 32×32 texel blocks of the reference on the GPU in a 2-bits-per-base×2-bits-per-base mesh used by the climbing algorithm. If the query is present in the reference sequence one or more times, then the algorithm reports the node contains the last character of the query. From this, the algorithm can report the number of occurrences and positions of the query in the reference in time proportional to the number of occurrences of the query in the reference. The CUDA architecture, a program can utilize textures for storing large read-only data, and reads from textures are cached using a proprietary 2D caching scheme, optimized for applying textures for graphics applications. Therefore, the algorithm optimizes the 2D locality of the matrix in these textures by organizing the nodes in 32×32 texel blocks.

Although it has been suggested that this so-called “climbing algorithm”, as designed by Wozniak (1997) for graphical display can be optimized by suppressing either the vertical or horizontal components of the diagonal array, this is not what we have found through empirical testing. FIG. 3 shows the diagonal parallelization used in the HUGEPOPS algorithm, although this algorithm does use the Smith and Waterman algorithm. Instead, HUGOPOPS extends the “global” sequence alignment of general global alignment technique in the Needleman-Wunsch algorithm that determines the distance of two sequences, using a novel dynamic programming method that is scalable to millions of human genome sequences, combining this approach with an anti-diagonal query matches to reference sequence. The method assumed that the length of the sequences in question are n and the total number of divisions are k=p+r. Using the sliding window-based climbing algorithm, the problem is defined as the horizontal division of the length 1=ⁿ, the probability of a random pattern of length n having p non-masked divisions exactly matching their counterparts in the read is shown below. In this case, we are comparing each selected query target against a reference genome, which can be defined as the latest version of the HuRef release, or the newer NCBI human reference genome sequence.

$${P}_{m} = ( \begin{matrix}k & … & k \\ ⋮ & ⋱ & ⋮ \\ p & … & p\end{matrix} ) {( \frac{1}{4} )}^{( \frac{16}{4} ) \cdot ( 2 )} = 6 \cdot {( \frac{1}{4} )}^{16}$$

The assumption is that the combined sequence length of all pre-selected target pharmacogenes will amount to less than 0.5% of the entire 3.2 bp length of the human genome in any batch run (<160,000,000 bp), so that the hypothetical number of random matches in this subset of the human genome is 1.6×10⁷. If you designate this as [symbol] , then the probability of a mismatch in this dataset is close to □, and the number of random matched sequences is <4.

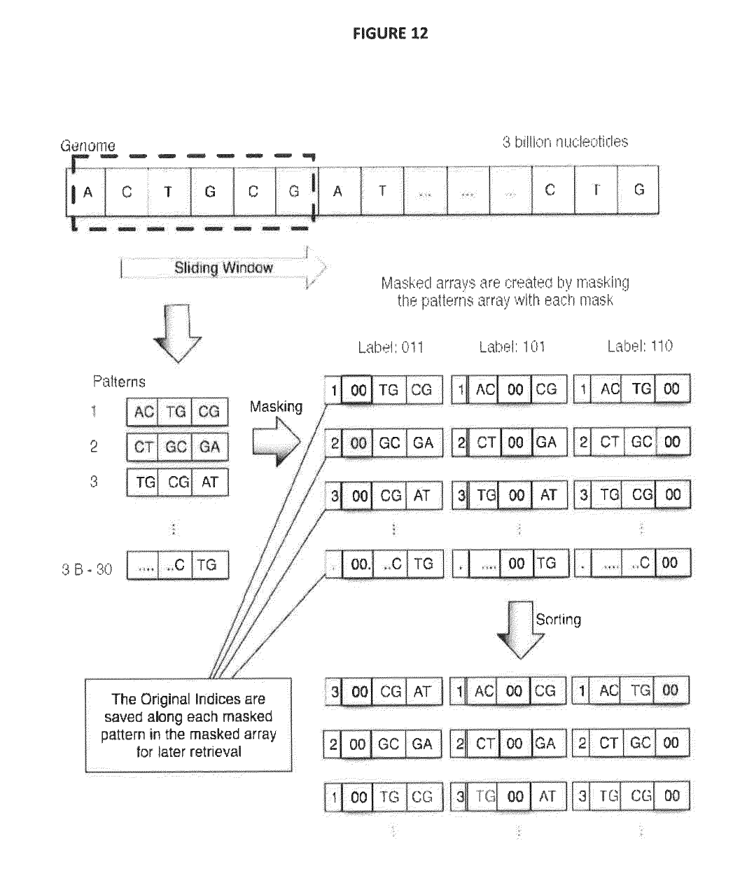

FIG. 12 shows the Pigeon hole filter associated with the sliding window algorithm. This is an instance where the sliding window with distributed filter (shown in FIG. 12) is based on the pigeon hole principle. In this example, pattern/reads are sought which are 1 mismatch apart. First, the pattern/reads are divided into 3 divisions. The pigeon hole principle states that at least one of divisions should be exactly matching. Leveraging this fact, the divisions can be masked that might have errors and a search is done for exact matches in the unmasked divisions. In this case, there are only three ways to mask one division out of the 3: 0FF, F0F and FF0.

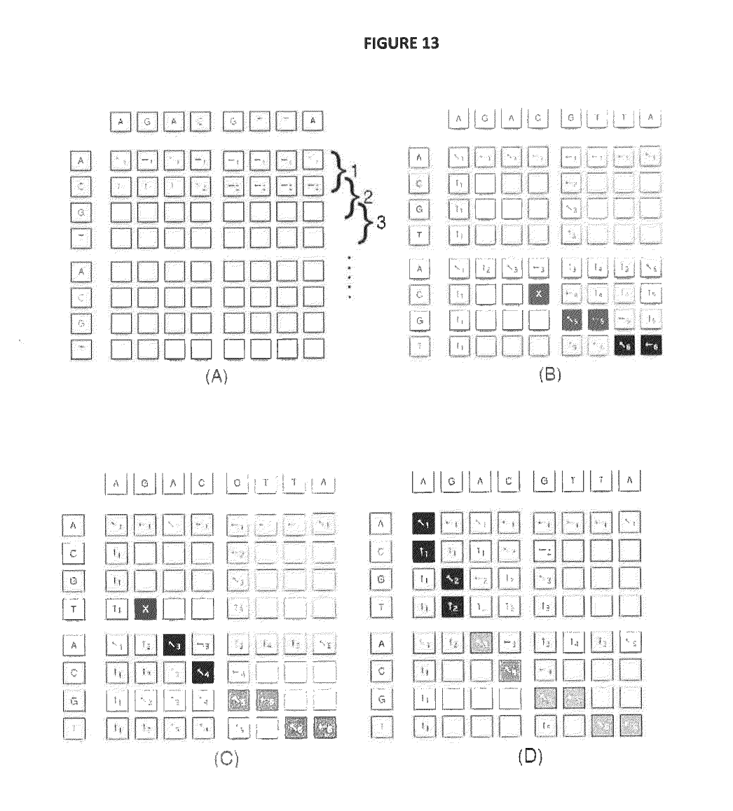

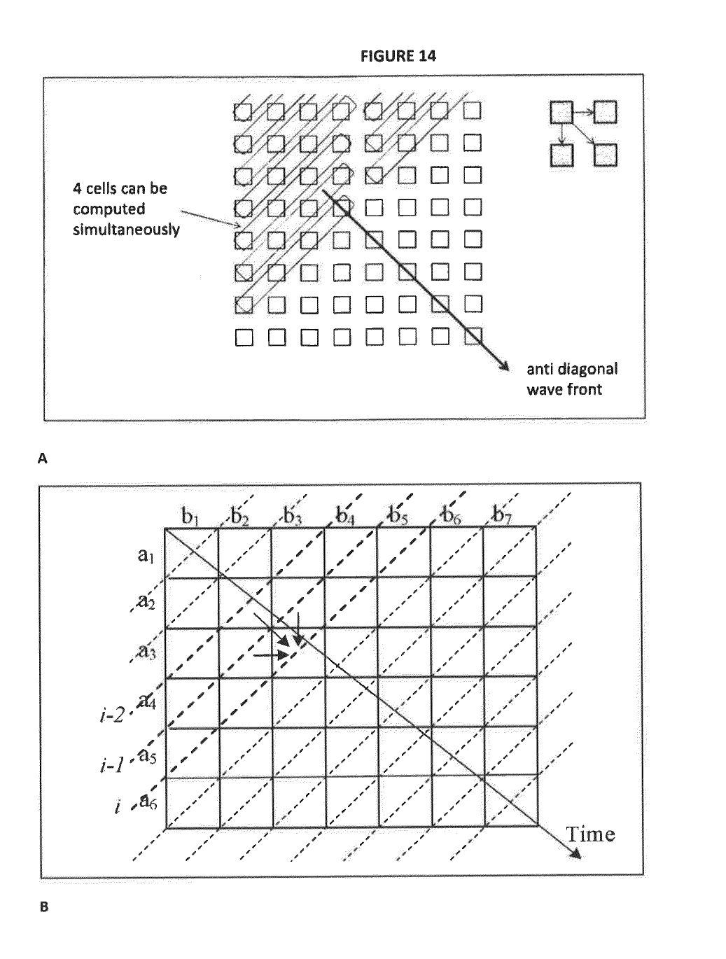

FIG. 13 shows the accurate alignment computation in the GPU for a 1×2 mesh. (A) The first pass of the algorithm keeps only two active rows of the alignment matrix while scanning it from top to bottom. During this scanning pass, it computes the boundary values of the smaller trivial quadrants for later access by the second pass of the algorithm, shown as shadowed cells in (B). (B) The second pass of the algorithm relies on the boundary values calculated in the previous pass. Having these values ready for each quadrant, we can start from the last quadrant and compute the inner values using a simple Needleman-Wunch dynamic programming variant. The algorithm then starts tracking back from the last element of the matrix and follows the directions to find the exit cell, denoted by letter ‘X’. (C) Keeping a record of the trace-back so far, it is continued in a new quadrant using the exit value of the previous quadrant. (D) The algorithm finally exits the larger alignment matrix through a quadrant either on the left edge or top edge of the alignment matrix. However, the method extends this approach by using an anti-diagonal wave front (See FIG. 14) with a speed-up of 180-fold over the approach used in FIG. 13, exploiting the ability of the texture memory to execute a diagonal mesh as shown in FIG. 14.

Using the same approach as shown in FIG. 13, FIG. 14 shows the HUGEPOPS algorithm performs both horizontal and vertical sliding window algorithms in parallel. There is no loss of speed, so neither horizontal nor vertical sliding windows dependencies need to be suppressed. In 3.1, as originally proposed by Wozniak (1997); In 3.2, as executed in HUGEPOPS, which employs a modification of the Needleman-Wunsch algorithm.

Algorithm execution:

```
      Parallel For-Loops to fill diagonal matrix with two sequences Seq1 and Seq2
      using two different threads per core
    For i=2 to Length of Data Array
      DataArray [0,i] = Seq1[i−2]
    For j=2 to Depth of Data Array
      DataArray [j,0] = Seq1[i−2]
1.2. Parallel For-Loops to fill diagonal matrix with two sequences (seq1 and seq2) using two
  different threads per core.
For-Loop
    For i=2 to Length of Pointer Array
      PointerArray [0,i] = Seq1[i−2]
    For j=2 to Depth of Pointer Array
      PointerArray [j,0] =Seq1[i−2]

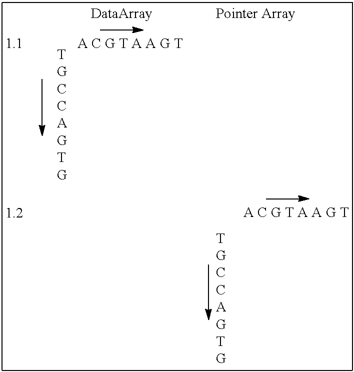

1.3 Initializing the anchor point of the diagonal Matrix
    DataArray [1,1] = 0
1.4 Parallel For-Loops to fill diagonal matrix with GAP values using two different GPU threads
executing each For-Loop
    Temp = 0
    For i=2 to Length of DataArray
    Temp = Temp + GAP
    DataArray [1,i] = Temp
    Temp = 0
    For j=2 to Depth of DataArray
    Temp = Temp + GAP
    DataArray [j,1] = Temp
duration1 = 1
For (loop1 = 0 ; loop1 < duration1 ; loop1++)
    itemp = 2
    jtemp = duration1
    For a = 0 to loop1
      str = itemp+,+jtemp
      newArr[loop1, a] = str itemp++
      jtemp--
      if (durationl < length)
      duration1++
    iitemp = length/2 + 1 duration2 = length/2 newI = length
For ( loop2 = duration2 ; loop2 >= 0 ; loop2--)
    itemp = iitemp jtemp = length
For (int a = loop2 ; a >= 0 ; a--) str = itemp+,+jtemp newArr[newI−1, a] = str itemp++
    jtemp—
    newI++
    iitemp++
    if (duration2 >= length)
      duration2—
1.5 Initializing the anchor point of the Pointer diagonal matrix
    PointerArray [1,1] = 0
1.6 Parallel For-Loops to fill Pointer diagonal matrix with GAP values using two different GPU
  threads executing each For-Loop
    Temp = 0
    For i=2 to Length of PointerArray
      Temp = Temp + GAP
      PointerArray [1,i] = Temp
    Temp = 0
    For j=2 to Depth of PointerArray
      Temp = Temp + GAP
      PointerArray [j,1] = Temp

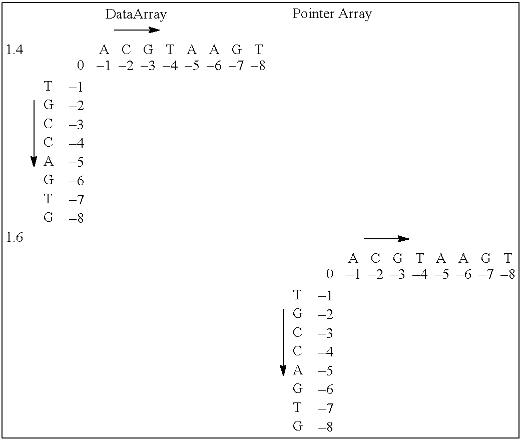

$No . of Threads = Ceil [ \frac{No . of values in the current diagonol}{Threshold [ Upper limit ]} ]$
Where Threshold is the range of values from which we
select the number of values to be solved per thread.
$Workload = Ceil [ \frac{No . of values in the current diagonal}{No . of Threads} ]$
Workload is the number of values to be solved per thread.
```

For each new diagonal, a new session is created. Each session consists of one or more threads depending on the length of the diagonal and the length of the query sequence. Each new session is independent of the results of any other session. As long as the threads of a session are running, an infinite number of sessions can be created, depending on the number of GPU cores that are available.

The method implements the distributed filtering scheme to find the right set of masks and distribute them across the computing nodes of the cluster. Once the masks are found, each ‘mapper’ program creates its corresponding set of masked arrays in the memory and starts processing through the reads one by one. If any read after being masked (and shifted in the process) can be matched in a masked array, it will be inserted in a buffer along with the matching pattern for further processing.

The implementation of the HUGEPOPS algorithm described herein involved many optimizations required to reduce the memory usage of each thread. Since the amount of computation per data input (and eventually output) is quite considerable, the computation is not memory bound, therefore we thrive to increase the utilization of the GPU to maximize the performance of this algorithm. The method calculates the maximum amount of register and shared memory available to the program for each thread for certain device occupancy.

The method uses a distributed filter to transform the non structured computational problem of finding all matches for each read into the reference sequence to a structured problem of pairs of potentially matching reads/patterns. The structured problem can then be delegated to a hardware accelerator, such as GPU, to accurately weed out all false positives. In the end, the results are accurate. There are neither false positives nor false negatives, and every SNP and MNP can be found using this window-sliding algorithm to a population frequency of 0.1%.

The next step in the method is to apply the ‘Sorting Tolerant From Intolerant’ (SIFT) multi-step algorithm that uses a sequence homology-based approach to classify amino acid substitutions that would occur based on SNPs or MNPs located in exons of selected targeted genes. SIFT, an open source program, detects non-synonymous single nucleotide polymorphisms (nsSNP) occurring in a coding gene that may cause an amino acid substitution in the corresponding protein product, thus affecting the phenotype of the host organism. Non-synonymous variants constitute more than 50% of the mutations known to be involved in human inherited diseases. This demonstrates the important role of the non-synonymous variation in human health and the strong effects it can have on an organism's phenotype. With ˜122,000 human nsSNPs in single nucleotide polymorphism database (dbSNP), a database of genetic variation hosted by the National Center for Biotechnology Information (NCBI) (http://www.ncbi.nlm.nih.gov/projects/SNP/), there is a significant need to characterize nsSNPs, with respect to their effect on the corresponding protein function.

The next step in the method is to apply the open-source PolyPhen-2 algorithm, which detects damaging mutations as a consequence of genome sequence variation in exons. PolyPhen-2 calculates Naïve Bayes posterior probability that this mutation is damaging and reports estimates of false positive (the chance that the mutation is classified as damaging when it is in fact non-damaging) and true positive (the chance that the mutation is classified as damaging when it is indeed damaging) rates. A mutation is also appraised qualitatively, as benign, possibly damaging, or probably damaging. The method chooses both HumDiv- and HumVar-trained PolyPhen-2. Diagnostics of Mendelian diseases requires distinguishing mutations with drastic effects from all the remaining human variation, including abundant mildly deleterious alleles. Thus, HumVar-trained PolyPhen-2 is first used for this task. Next, the HumDiv-trained PolyPhen-2 is be used for evaluating rare alleles at loci potentially involved in complex phenotypes, where even mildly deleterious alleles must be treated as damaging. Scores are entered into the database.

The next step in the method is to calculate allele frequencies of the novel SNPs and MNPs that were detected by this invention. A modification of the Expectation-Maximization algorithm, first described for large populations by Excoffier and Slatkin (1995) is executed, with the following changes: For allele frequency estimation, there is not an assumption of 2 equal frequencies, and the process is repeated in a looped, iterative and redundant manner. Although the E-M algorithm is iterative, the iterative process is maximized.

Finally the method reports all SNP and MNP polymorphisms to an indexed database with classification such that post-processing of resultant data can be assessed to understand selected target variant sequences. From this massed sequence data, detailed examination of human population genomics can be performed, and sequences can be tested in trials to determine the clinical utility of sequence polymorphisms that can inform a molecular diagnostic test.

The present invention provides a method of compiling, aggregating and performing a concordance analysis, including reference to the latest NCBI release 52, of thousands of complete whole human genomes, said sequences generated by different sequencing technologies. The method exploits recent advances in information technology; combining fast file downloads (e.g., PGON) and/or data transfer using high speed, large capacity solid state storage (e.g., Express Card 2.0 PCI) to a GPU-cluster personal computer workstation optimized to provide over 8 Teraflops of compute speed for data processing executed in CUDA “Fermi” architecture. CUDA is the most advanced GPU computing architecture with over three billion transistors and featuring up to 512 CUDA cores. A workstation configured in the manner disclosed in this invention supports supercomputing performance at 10% of the cost a traditional CPU-only server and at 0.1% of the power requirements of a single GPU-cluster server located in an institutional datacenter. The method involves conversion of different file formats to a uniform file format that can be used in other parts of the invention, relying on the ease of use and efficiency of the AGP 2.0 file format conversion. The method also provides a mode in which a user may select targeted gene coordinates using common genome browsers for subsequent enrichment. The method also provides a process to extract only selected pharmacogenes and flanking regions that include vital regulatory sequences. The method also provides a mechanism to perform multi-genome variant analysis and validation of common and rare SNPs and MNPs, whose output can be used to configure pharmacogenic-based diagnostic tests in medicine.

The present invention also provides a method of performing human population genomics in epidemiology. The method accepts completed whole genomes that can be identified as to disease phenotype, endophenotype, ethnicity, age, gender and other characteristics. The compiling and aggregation module records and stores annotated data such as these descriptors, as well as sequence data. The selection process is particularly useful for genomic analysis of a complex human population, with regards to disease risk and drug response, and lends itself to rapid determination of those subpopulations or individuals that may be at greatest danger to an acute or chronic environmental event that may impact the individual based on its genome polymorphisms.

The present invention can relate to configuration of an inexpensive and powerful workstation that can be made portable for deployment for genome research in hospitals, reference and commercial diagnostic laboratories, academic medical centers, pharmaceutical and biotechnology companies, for fast determination of selected, targeted genes for polymorphism analysis. The process of supporting genome sequence data in a secure cloud environment negates the purchase of expensive, costly and energy inefficient servers for database access.

The present invention additionally provides a method for making a population of selection probes to be used for life science research, clinical research and other applications. The selection probes are particularly useful if they are a subset of a complex population. For example, a particularly useful population of selection probes would be derived from a subset of complete whole genomes for identification of an individual in forensic science.

The present invention provides novel single nucleotide polymorphisms (SNPs) and multiple polynucleotide polymorphisms (MNPs) located in various target pharmacogenes and methods of using these SNPs and MNPs to determine response to treatment (e.g., of a psychotropic disorder or depression) or determine the potential for adverse events in response to therapeutic strategies.

The skilled artisan, reading the present application would recognize that the specific location of the disclosed SNPs and MNPs in the complete sequences (exon and/or intron sequences) of the pharmacogenes described herein can be assessed and determined, without undue burden, using widely acceptable and readily available websites to access genome sequence data (e.g., UCSC Genome Browser, Integrative Genomics Viewer, Ensemble, Genbank etc.).

Table 2 shows the analysis of selected pharmacogenes in 17,131 whole genomes

| | | Novel SNP(s) | | | Number of | Vali- |
|---|---|---|---|---|---|---|
| | | Number in | Number in | Novel | Replicated | da- |
| | Gene | exons | introns | MNP(s) | Runs | tion |
| 1 | ABCB1 | 15 | — | — | 6 | Yes |
| 2 | ADCYAP1R1 | 1 | 4 | 1 | 6 | Yes |
| 3 | ADRA2A | 2 | — | 1 | 6 | Yes |
| 4 | BDNF | 2 | — | — | 6 | Yes |
| 5 | COMT | 3 | — | — | 6 | Yes |
| 6 | CHRHBP | 1 | — | — | 3 | Yes |
| 7 | CRHR1 | 5 | — | — | 6 | Yes |
| 8 | DBI | 18 | — | 2 | 6 | Yes |
| 9 | DRD2 | 5 | — | — | 6 | Yes |
| 10 | DRD4 | 3 | 1 | — | 6 | Yes |
| 11 | FKBP5 | 10 | — | — | 6 | Yes |
| 12 | GCR | 7 | — | — | 6 | Yes |
| 13 | HTR2A | 5 | 3 | — | 6 | Yes |
| 14 | HTR2C | 1 | — | 2 | 6 | Yes |
| 15 | MAOA | — | — | — | 6 | Yes |
| 16 | NPY | 2 | — | — | 6 | Yes |
| 17 | NT3 | 4 | 3 | — | 6 | Yes |
| 18 | NTRK2 | 10 | — | — | 6 | Yes |
| 19 | OPRM1 | 3 | — | 1 | 6 | Yes |
| 21 | SKG1 | — | — | — | 6 | Yes |
| 22 | SLC6A2 | 2 | — | 2 | 6 | Yes |
| 23 | SLC6A3 | 12 | — | | 6 | Yes |
| 24 | SLC6A4 | 8 | 2 | 1 | 7 | Yes |

| | | | |
|---|---|---|---|
| TOTAL | 121 | 13 | 10 |

TABLE 3
Shows exon SNPs detected by the invention, and their frequencies and putative
deleterious consequences.

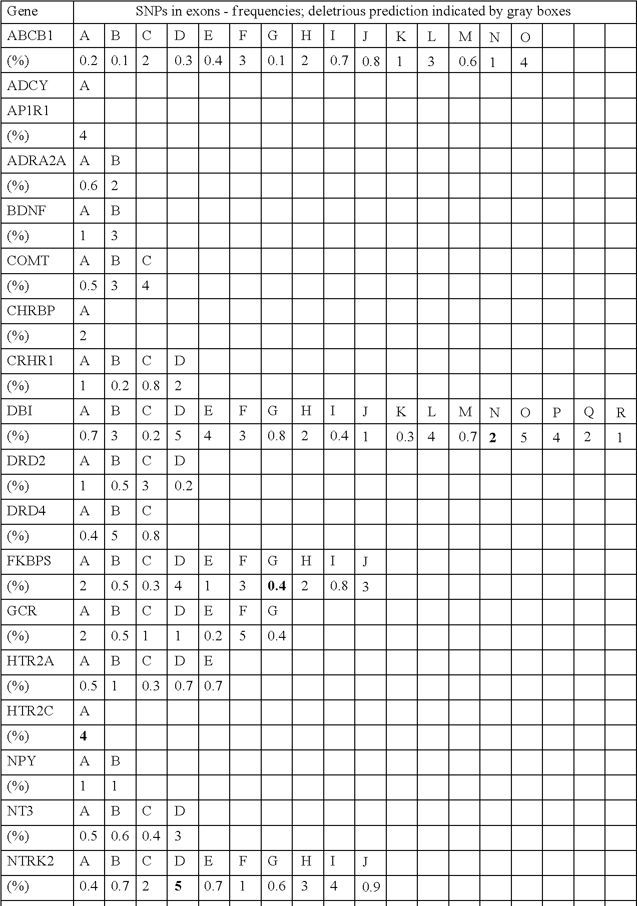

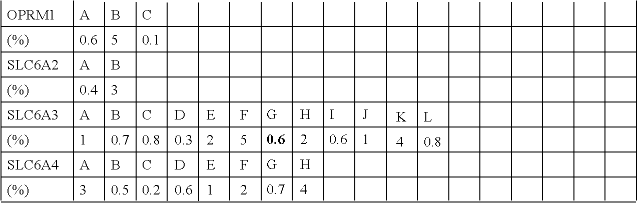

### ABCB1 (HGNC Nomenclature)

The delivery of drugs to the brain is hindered by the physiological interface separating the CNS from its vascular supply—the blood-brain barrier (BBB). As a consequence, the BBB is the major rate-limiting step for drug distribution to different brain regions. One of the major hurdles that inhibit drug permeability is the super-family of ATP-binding cassette (ABC) proteins, including ABCB1, and some of these 49 proteins convey multidrug resistance (MDR) to the BBB. In the central nervous system (CNS), most ABC transporters are oriented to expel drugs in one direction into the blood, but not into the cerebrospinal fluid (CSF). For psychotropic drugs, ABCB1 acts as a major gatekeeper at the BBB1. There is extensive literature regarding ABCB1 gene variants and “multi-drug” resistance. The ABCB1 gene encodes P-glycoprotein (P-gp), a major efflux transporter protein that traverses not only the BBB, but also the endothelial lining of the gastrointestinal system and urinary system. So, it is important to recognize that ABCB1 variants may influence access of psychotropic drugs, both to CNS targets and/or by limiting absorption through the lining of the gut.

Structure of the ABCB1 Gene: The term ABC transporter was introduced by Christopher Higgins in 1992. The name is based on the highly conserved ATP-Binding Cassette, which includes 49 genes in human that have been identified to date. The gene is located on Chromosome 7: 87,133,175-87,342,564. Analysis of human cell lines, liver tissue, and lymphocytes consistently show ABCB1 to contain 29 exons in a genomic region spanning 209.6 kb. The ABCB1 promoter region contains a few low-frequency polymorphisms and is relatively invariant compared to other genes in the genome. The numbering of exons reflects the fact that the ABCB1 gene can be transcribed from two different promoters, an upstream promoter and a downstream promoter, the latter being preferentially expressed in most cell lines. The upstream promoter is found at the beginning of exon-1, and the downstream promoter is located within exon 1. The ATG translation initiation codon is located within exon 2. Thus the protein-coding sequence of the ABCB1 gene comprises 27 exons, 14 of which encode the first half and 13 encode the second half of the protein. There are 28 introns, 26 of which interrupt the protein-coding sequence. The human ABCB1 gene does not have a TATA box in the promoter, but instead contains an initiator element (Inr) defined by the consensus Py-Py-A(+1)-N-(T/A)-Py-Py. In the absence of a TATA box, initiator elements direct basal transcription and also ensure accurate transcriptional initiation. Transient transfection studies reveal that the sequence between −6 and +11 bp is sufficient for proper initiation of transcription. A recent study showed that NF-κB and CREB are the most profound protein regulators of ABCB1 gene expression. The messenger RNA (mRNA) of ABCB1 is 4872 base pairs in length, including the 5′ untranslated region (UTR), which gives rise to a protein that is 1280 amino acids in length, named P-glycoprotein (P-gp). The secondary structure of P-gp reveals two homologous halves to the protein, each containing six transmembrane domains and a nucleotide-binding domain. The existence and number of putative splice variants is as yet undetermined. Alternative transcripts for ABCB1 have been predicted from sequence alignments with human complementary DNA (cDNA). The human brain expresses the most transcripts of any human tissue, with 19 identified.

ABCB1 Polymorphisms: There are several hundred SNPs in the large ABCB1 gene. Less than 100 SNPs have been identified in the coding region; more are contained in the 5′UTR and 3′UTR, and within introns. Fifty-three new SNPs have been recently found by deep-sequencing of 18.5 kb of the ABCB1 gene to a coverage of 30-fold or greater. These more recently discovered variants are rare, and have not been examined in association with psychotropic drug response. The first systematic investigation on ABCB1 SNPs revealed a significant correlation of a silent polymorphism in exon 26 (3435C>T; rs1045642) with intestinal P-gp expression levels and oral bioavailability of digoxin, showing significantly decreased intestinal P-gp expression and increased digoxin plasma levels after oral administration among homozygote 3435TT carriers. The frequency of the putatively most interesting 3435C>T SNP differs significantly between ethnicities. The variant 3435TT allele has a prevalence of 0.03 in Africans, 0.20-0.24 in Oriental populations, and 0.31-0.34 among Caucasians. Such genotypic differences may contribute to interethnic differences of drug responses in certain populations. Three single nucleotide polymorphisms (SNPs) occur frequently and exhibit strong linkage disequilibrium, creating a common haplotype at positions 1236C>T (rs1128503), 2677G>T (rs2032582) and 3435C>T (rs1045642). This common haplotype is mentioned in some of the association data. Recent studies show that variations in this haplotype block is responsible for most CNS drug response in humans, but it is not rs1045642 that is responsible, but rather rs2032582.

Data from PharmGkb.org on ABCB1 haplotypes is shown in Table 4.

TABLE 4

| | TYPE/ | STRENGTH OF | | |
|---|---|---|---|---|
| SNP | EFFECT | EVIDENCE* | DRUG | DISEASE |
| rs2032582 | Efficacy | 2 | efavirenz, nelfinavir | HIV |
| rs2032582 | Efficacy | 2 | cytarabine, idarubicin | Acute myeloid leukemia |
| rs2032582 | Toxicity/ADR | 2 | carboplatin, cisplatin, | Ovarian Neoplasms |
| | | | docetaxel, paclitaxel, | |
| | | | taxanes | |
| rs2032582 | Toxicity/ADR | 2 | Platinum compounds, | Ovarian Neoplasms |
| | | | taxanes | |
| rs2032582 | Efficacy | 2 | anthracyclines and related | Breast Neoplasms |
| | | | substances | |
| rs2032582 | Efficacy | 2 | paclitaxel, taxanes | Breast Neoplasms |
| rs1045642 | Toxicity/ADR | 3 | prednisone, tacrolimus | |
| rs1045642 | Efficacy | 3 | methotrexate | Arthritis, Rheumatoid |
| rs1045642 | Dosage | 3 | fexofenadine | |
| rs1045642 | Efficacy | 3 | anthracyclines and related | |
| | | | substances, | |
| | | | cytarabine, doxorubicin, | |
| | | | epirubicin, idarubicin | |
| rs1045642 | Toxicity/ADR | 3 | nortriptyline | Major Depressive |
| | | | | Disorder, |
| | | | | Hypotension |
| rs1045642 | other | 3 | lansoprazole, tacrolimus | Gastroesophageal |
| | | | | Reflux, Transplantation |
| rs1045642 | Toxicity/ADR | 3 | nevirapine | HIV Infections |
| rs1045642 | Efficacy | 3 | anthracyclines and related | Breast Neoplasms |
| | | | substances, taxanes | |
| rs2229109 | Other | 3 | vinblastine | |
| rs2229109 | Other | 3 | paclitaxel | |
| rs2229109 | Other | 3 | verapamil | |
| rs2229109 | Other | 3 | prazosin | |
| rs2229109 | Other | 3 | forskolin | |
| rs2229109 | Other | 3 | calcein | |
| rs2229109 | Other | 3 | bisantrene | |
| rs9282564 | Other | 3 | verapamil | |
| rs9282564 | Other | 3 | prazosin | |
| rs9282564 | Other | 3 | forskolin | |
| rs9282564 | Other | 3 | calcein | |
| rs9282564 | Other | 3 | paclitaxel | |
| rs9282564 | Other | 3 | vinblastine | |
| rs9282564 | Other | 3 | bisantrene | |
| rs72552784 | Other | 3 | vinblastine | |
| rs72552784 | Other | 3 | paclitaxel | |
| rs72552784 | Other | 3 | prazosin | |
| rs72552784 | Other | 3 | forskolin | |
| rs72552784 | Other | 3 | calcein | |
| rs72552784 | Other | 3 | bisantrene | |
| rs72552784 | Other | 3 | verapamil | |

*Strength of Evidence: (2) p < 0.05 after error correction and at least 1 replicated study of >100 participants. (3) One study, either in vivo or in from in vitro data.

ABCB1 Polymorphism Nomenclature: In recent years, the bulk of published studies have adopted the gene nomenclature used throughout the National Center for Biotechnology Information (NCBI) databases. For example, the HUGO nomenclature of the National Human Genome Research Institute (NHGRI) must be used by all grant recipients of federal funding, and defines the standard for the nomenclature of genes, their products and genetic variants. The rs1045642 SNP shows the greatest ethnic variation of all of the ABCB1 SNPs studied to date. Since it is a functional SNP, it will certainly show heterogeneity in psychotropic drug response, depending on the subpopulation being studied. Multiple studies have demonstrated the following:

Allele and genotype frequencies of the 3435C>T SNP (rs1045642) according to ethnicity are shown in Table 5.

TABLE 5

| | | | | Genotype | | |
|---|---|---|---|---|---|---|
| | Summed | Allele | | Frequencies | | |
| | Sample Size | Frequencies | | (averaged - no | | |
| | (n = number | (averaged) | | range provided) | | |
| Ethnicity | of studies) | C | T | CC | CT | TT |
| African | 861 (9) | 82% | 18% | 66% | 31% | 3% |
| South | 1125 (6) | 60% | 40% | 34% | 35% | 31% |
| American | | | | | | |
| Asian | 3501 (27) | 49% | 51% | 29% | 47% | 24% |
| Indian | 115 (3) | 41% | 59% | 18% | 61% | 21% |
| South Asian | 124 (4) | 58% | 42% | 32% | 48% | 20% |
| Middle East | 396 (2) | 61% | 39% | 41% | 42% | 17% |
| Caucasian | 7225 (36) | 44% | 56% | 22% | 44% | 34% |

Association of 3435C>T (rs1045642) with Clozapine Response: Consoli, et al. Pharmacogenomics. 10(8):1267-76 (2009) examined clozapine and norclozipine plasma levels, as well as clozapine response, in a small sample of psychotic Caucasian patients. They examined carriers of 3 SNPs: 3435C>T (rs1045642); 1236C>T (rs1128503) and 2677G>T (rs2032582). The authors tested for HWE, with a frequency of wild type alleles at 45% (rs1045642), 54% (rs1128503) and 55% (rs2032582) for SNPs on exons 26, 21 and 21 respectively. Patients with 3435CC or 2677GG genotypes had significantly lower dose-normalized clozapine levels than those who were heterozygous or TT carriers.

An important finding was that psychotic patients that were carriers of 3435CC (n=15) required higher clozapine doses to achieve the same plasma concentrations as CT or TT patients. They required significantly higher doses of clozapine to reach the same clinical benefit, 246+142 mg/day versus 140+90 mg/day for 24 CT and 21 TT patients. Although the sample size of this study was small, there appears to be an effect in Caucasians where the 3435CC genotype makes them more resistant to clozapine. This effect might be mediated through gene-gene interactions with CYP450 enzymes, a change in substrate, or through increased expression of P-gp.

Association of 3435C>T rs1045642 with Antidepressant Drug Response Side Effects: Roberts, et al. Pharmacogenomics J. 2(3):191-6 (2002) examined this SNP in Caucasian patients with major depression enrolled in a randomized antidepressant treatment trial of nortriptyline and fluoxetine, and observed a significant association between nortriptyline-induced postural hypotension and 3435C>T (chi2=6.78, df=2, P=0.034). Their results suggest that the 3435TT allele of ABCB1 is a risk factor for occurrence of nortriptyline-induced postural hypotension (OR=1.37, P=0.042, 95% CI 1.01-1.86). This study suggests that use of nortripyline by Caucasian carriers of the 3435TT genotype is more likely to experience postural hypotension as a side effect of antidepressant use.

Efficacy: In Fukui, et al. Ther. Drug Monit. 29:185-9 (2008), the C3435T SNP was investigated and shown to affect mean fluvoxamine plasma concentration. This study involved 62 Japanese outpatients, of which 55 were diagnosed with major depressive disorder. Subjects were given fluvoxamine in 50 mg/day increments up to a 200 mg/day dosage. Serum levels were obtained after 2 weeks on the same dosage in order to obtain steady state levels. Significant association between plasma concentration and 3435TT genotype was observed at the 200 mg/day dosage, but not at the 150 mg/day, 100 mg/day, or 50 mg/day dosages. In Asian patients, the 3435TT genotype seems to define a poor metabolizer phenotype.

Lin, et al. Pharmacogenet. Genomics. 21(4):163-70 (2011) examined 28 ABCB1 SNPs and their association with Major Depressive Disorder and remission following treatment with escitalopram. The study included 100 patients of Asian ethnicity, and examined metabolites of escitalopram at weeks 2, 4 and 8. They found significant association of the ABCB1 SNPs rs192242 (p=0.0028) and rs1202184 (p=0.0021) with the severity of depressive symptoms as assessed by the Hamilton Rating Scale for Depression adjusted with Hamilton Rating Scale for Anxiety. Importantly, they found that that the haplotype block rs1882478-rs2235048-rs1045642-rs6949448 (from intron to intron 26) was strongly correlated with remission rate following escitalopram treatment (global p=0.003). More detailed analysis showed that carriers of the 3435TT (rs1045642) genotype were associated with a slower remission on the antidepressant (p=0.001). In Asian patients, the 3435TT genotype seems to convey treatment resistance to escitalopram.

Kato, et al. Prog. Neuropsychopharmacol. Biol. Psychiatry 32:398-404 (2008) examined 3 functional polymorphisms, including (C3435T: rs1045642, G2677T/A: rs2032582 and C1236T: rs1128503) with response to paroxetine in a Japanese major depression sample (62 patients) followed for 6 weeks. Analysis of covariance at week 6 with baseline scores included in the model as covariate showed significant association of the non-synonymous SNP G2677T/A with treatment response to paroxetine (p=0.011). In contrast, the haplotype block (3435C-2677G-1236T) resulted associated with poor response (p=0.006). On further analysis, the 3435TT genotype accounted for the majority of this poor response to paroxetine (p=0.0008). The authors noted that the variants were not in linkage disequilibrium as strong as previously reported, which they attributed to the small sample size used in this study. In Asian patients, the 3435TT genotype seems to convey treatment resistance to paroxetine.

Uhr, et al. Neuron. 57:2039 (2008) examined the association of multiple ABCB1 SNPs in a large Caucasian population. Patients were subdivided into two groups according to the antidepressant property as P-gp substrate. Patients taking antidepressants that are substrates of P-gp received amitriptyline, paroxetine, venlafaxine, or citalopram, and patients taking antidepressants that are not substrates of P-gp received mirtazapine for at least 4 weeks. Trained raters using the 21 item HAM-D scale assessed the severity of psychopathology at admission. Patients fulfilling the criteria for at least one moderate depressive episode (HAM-D R 14) entered the analysis. Remission was defined as reaching a total HAM-D score of less than 10. All highly associated SNPs were located in introns and with the exception of rs2235015 located in a single haplotype block. However, upon further examination, the genotype 3435TT (rs1045642) showed an association (p=0.044) with response at week 5 in grouped (substrate and non-substrate) data. Although intronic sequences were most closely associated with P-gp substrate-based, antidepressant response, carriers of the 3435TT genotype showed a positive effect correlated with antidepressant drug response in this study.

Interaction between the ABCB1 3435C>T SNP and CYP2D6*10/*10 Metabolizers. Yoo, et al. Br. J. Pharmacol. 164, 433-443 (2011) studied the pharmacokinetics of risperidone according to genetic polymorphisms in CYP2D6 and ABCB1 (3435C>T and 2677G>T/A) in a population of healthy subjects (n=72) who were administered 2 mg of the drug. There were no significant differences in the AUC of risperidone in the ABCB1 3435C>T genotypes. Unlike the single 3435C>T genotypes, carriers of the 3435TT genotype in individuals with the CYP2D6*10/*10 genotype were associated with statistically significant differences in the pharmacokinetic parameters of risperidone—the AUC of risperidone was significantly (P=0.001) higher in 3435TT subjects than in 3435CC subjects who were CYP2D6*10/*10. If the P-gp transporter and CYP2D6 enzyme sequentially and independently affect the disposition of risperidone, the pharmacokinetic parameters of risperidone will mostly be dependent on the enzymatic activity of CYP2D6, and the metabolic ratio of risperidone will not change with the ABCB1 activity. The metabolic ratios of risperidone were significantly (P=0.004) associated and changed with the 3435TT genotype groups with CYP2D6*10/*10. Moreover, the metabolic ratios of risperidone were significantly (P=0.006) higher in 3435TT than in 3435CC with CYP2D6*10/*10. These results showed that the influence of genetic polymorphisms in the ABCB1 and CYP2D6 genes on the pharmacokinetics of risperidone was combined, and that the interplay of P-gp and CYP2D6 enzymes may play an important role in the disposition of risperidone. The CYP2D6*10/*10 genotype is a major variant in Asians, and is associated with decreased CYP2D6 activity resulting from the formation of an unstable enzyme. Approximately 50% of Koreans carry this allele, whereas only 2% of Caucasians carry this genotype.

Epistasis: Studies using direct sequencing have revealed additional SNPs that had not been previously assessed in association studies. For example, in a multi-gene study targeting the various genes involved in the pathway of antidepressant drug response in Mexican-Americans with Major Depressive Disorder (MDD), the investigators re-sequenced seven candidate genes of importance in the pathophysiology of the disease. Using a hypothesis-driven, targeted deep sequencing approach, the study looked at a group of genes that reflected a succession of events relevant to drug action at four levels: (1) Entry of the antidepressant drug into the brain (ABCB1); (2) Binding of the drug to monoaminergic transporters (SLC6A2, SLC6A3 and SLC6A4); (3) Distal effects at the transcription level (CREB1—regulates ABCB1 gene transcription); and (4) Subsequent changes in neurotrophin and neuropeptide receptors (neurotrophic tyrosine kinase type 2 receptor (NTRK2), important in synaptic function and neural plasticity, and corticotropin-releasing hormone receptor 1 (CRHR1), which regulates the HPA axis). Using this approach, the researchers found an additional 28 SNPs in the ABCB1 gene that had not been previously identified, and thus had not been investigated in previous association studies (see Table 6). In addition to the 28 new SNPs discovered in the ABCB1 gene through the use of direct sequencing and analysis, they found a total of 204 new SNPs in all 7 genes that had never been found. Given the small size of the study (n=272), and the need to use a statistical correction method for multiple associations, no significant associations between the known SNPs or the newly discovered ones revealed strong association with disease or antidepressant drug response.

TABLE 6
Deep sequencing reveals additional SNPs in the ABCB1 gene that may be involved in antidepressant response:

| SNP | Downstream | 3′ UTR | Intron | 5′ UTR | Upstream | Synonymous | Nonsynonymous | ALL | Sequence |
|---|---|---|---|---|---|---|---|---|---|
| NEW | 0 | 0 | 20 | 4 | 0 | 1 | 3 | 28 | 18.5 kb |
| dbSNP | 0 | 4 | 37 | 4 | 1 | 2 | 5 | 53 | |
| TOTAL | 0 | 4 | 57 | 8 | 1 | 3 | 8 | 81 | |

*Synonymous SNPs; Those nudeotide substitutions that do not change the amino acid (due to wobble); Nonsynonymous SNPs: Nucleotide substitutions that result in a change to the amino acid.

Summary: From these studies, the ABCB1 SNP 3435C>T (rs1045642) seems to have an association with clozapine response in Caucasians, with the 3435CC genotype conveying some degree of drug resistance. For antidepressant drugs, the 3435TT genotype in Asians administered fluvoxamine, escitalopram and paroxetine showed significant treatment resistance. In Asians with CYP2D6*10/*10 and ABCB1 3435TT genotypes had significantly elevated metabolic rates compared with the combination of CYP2D6*10/*10 and ABCB1 3435TT genotypes. This is significant in Asians, but probably not in Caucasians, because of the low frequency of the CYP2D6*10/*10 allele in Caucasians. Preliminary data suggest that the 3435TT (rs1045642) genotype in Caucasians shows an association with a broad spectrum of antidepressant drugs, whether they are substrates of P-gp (e.g., amitriptyline, paroxetine, venlafaxine, or citalopram) or not. The physiological consequences of ABCB1 transporter genetic variants are still only partly understood. The overall bioavailability of drugs seems to be only moderately influenced by the currently known ABCB1 SNPs, at least as compared to variants of the CYP450 system, with the ABCB1 3435C>T SNP having the greatest impact—although this may be a “marker” SNP for rs2032582, which is located in the same haplotype block. It is interesting to note that among bioavailability studies performed in Caucasians, 3435TT carriers presented higher plasma concentrations, whereas among Asians this was the case for 3435CC subjects, indicating possible different haplotype clusters in these ethnicities. Finally, although the 3435C>T genotype frequency difference is most pronounced in Africans and African-Americans, no studies have been undertaken in these populations with regard to ABCB1 SNPs and psychotropic drug response. Further studies are required to define the relationship of ABCB1 SNPs and psychotropic response.

The results of this invention detected all of the known, validated SNPs contained in the dbSNP database as of Apr. 20, 2012 (http://www.ncbi.nlm.nih.gov/projects/SNP), but also found other, more rare SNPs that showed concordance across all 3 sequencing platform outputs. The novel SNPs listed as M, N and O in Table 7 below are in the same haplotype block as rs2032582. None had putative effects on the translated protein, as predicted by SIFT and PolyPhen 2 scoring.

The REF SEQ ID (GRCh37.p5) is incorporated herein by reference.

TABLE 7
Novel SNPs in the ABCB1 exons that may impact
drug response.

| | | | | SEQ |
|---|---|---|---|---|
| | | | | ID |
| | SNP | Position | MAF | NO: |
| A | AGAGGTG C/G AACGGAAGC | chr7: 87,342,572 | 0.2% | 1 |
| B | TCCGGGCC G/C GGAGCAGT | chr7: 87,342,870 | 0.1% | 2 |
| C | AAGGG G/A CCGCAATGGAG | chr7: 87,229,528 | 2% | 3 |
| D | ATACTATC T/A TCATTTACT | chr7: 87,190,712 | 0.3% | 4 |
| E | ACAAA A/T GAAAGAACTT G | chr7: 87,190,565 | 0.4% | 5 |
| F | GGGTGTAAGT G/C AG | chr7: 87,193,455 | 3% | 6 |
| G | GATACTGGCCCA A/T A | chr7: 87,192,683 | 0.1% | 7 |
| H | GCAT T/A TGCAAATGCAAG | chr7: 87,179,992 | 2% | 8 |
| I | ATCT T/A GAAGGGTCTGAA | chr7: 87,179,617 | 0.7% | 9 |
| J | CAGGTGGCTCT G/C GATAAG | chr7: 87,178,658 | 0.8% | 10 |
| K | CTAGAAGGTT C/G GGGAAG | chr7: 87,160,603 | 1% | 11 |
| L | ATTTTCAG C/G | chr7: 87,145,992 | 3% | 12 |
| | TGTTGTCTTTG | | | |
| M | TGACTATGC C/G AAAGCCAAA | chr7: 87,145,911 | 0.6% | 13 |
| N | GTGGGCAG C/G AGTGGCTGTG | chr7: 87,144,615 | 1% | 14 |
| O | ATTGCCAT A/ | chr7: 87,135,290 | 4% | 15 |
| | TGCTCGTGCCCTTG | | | |

ADCYAP1R1

The adenylate cyclase activating polypeptide 1 (pituitary) receptor type I, also known as the PACAP receptor, is a seven trans-membrane protein that produces at least seven isoforms by alternative splicing. Each isoform is associated with a specific signaling pathway and a specific expression pattern. The PACAP receptor, which is thought to play an integral role in brain development, and preferentially binds PACAP in order to stimulate a cAMP-protein kinase A signaling pathway. The endogenous ligand, PACAP, also activates the VIP receptors, VPAC1 and VPAC2. PAC 1 receptors are predominantly expressed in the central nervous system, particularly in the olfactory bulb, thalamus, hypothalamus, dentate gyrus and granule cells of the cerebellum. They are also found in the adrenal medulla and pancreas. PACAP receptors are involved in daytime regulation of the biological clock, emotional control of behavior, anxiolysis and control of adrenal medulla catecholamine release. The human ADCYAP1R1 gene has been localized to chromosome 7p14, 31, 092, 076-31, 151, 089.

ADCYAP1R1SNP rs2267735 and PTSD in female African-Americans: Pituitary adenylate cyclase-activating polypeptide (PACAP) is known to broadly regulate the cellular stress response. In contrast, it is unclear if the PACAP/PAC1 receptor pathway has a role in human psychological stress responses, such as posttraumatic stress disorder (PTSD). A single SNP in an estrogen response element within ADCYAP1R1, rs2267735, predicts PTSD diagnosis and symptoms in females only. This SNP also associates with fear discrimination and with levels of ADCYAP1R1 messenger RNA expression in human brain. Previous studies found that in heavily traumatized female subjects, there was a significant sex-specific association of PACAP blood levels with fear physiology, PTSD diagnosis and symptoms in females (N=64, replication N=74, p<0.005). Using a tag-SNP genetic approach (44 single nucleotide polymorphisms, SNPs) spanning the PACAP (ADCYAP1) and PAC1 (ADCYAP1R1) genes, they found a sex-specific association with PTSD, rs2267735, a SNP in a putative estrogen response element (ERE) within ADCYAP1R1, predictive of PTSD. Thus, their data suggest that PACAP/PAC1 receptor expression and signaling may be integrally involved in regulating the psychological and physiological responses to traumatic stress. Further, the finding of an association of an estrogen responsive element—embedded ADCYAP1R1SNP with PTSD, is consistent with the “glucocorticoid hypothesis of PTSD”, with fear- and estrogen-dependent regulation of PACAP systems within stress-responsive regions of the brain. These data may begin to explain sex-specific differences in PTSD diagnosis, symptoms, and fear physiology. Future work targeting the PACAP/PAC1 receptor system may lead to novel and robust biomarkers as well as to further our understanding of the neural mechanisms underlying pathological responses to stress with potential therapeutic targets towards the prevalent and debilitating syndrome of PTSD.

The results of this invention detected all of the known, validated SNPs contained in the dbSNP database as of Apr. 20, 2012 (http://www.ncbi.nlm.nih.gov/projects/SNP), but also found other, more rare SNPs that showed concordance across all 3 sequencing platform outputs. The novel SNP is listed as A in Table 9 below. It did not have putative effects on translated protein, as predicted by SIFT and PolyPhen 2 scoring. However, as demonstrated in Example 2, a MNP was identified that interfered with the ERE in the wild type ADCYAP1R1 sequence. Because of the large sample size of whole genomes available, a test was performed of the known SNP found to be associated with PTSD by ethnicity, by performing a test of the female and ethnically-identified cohort against rs2267735 SNP at chr7:3, 108, 667-31, 117, 836, to determine allele frequency in the population. The results are shown below in Table 8.

TABLE 8
ALLELE FREQUENCY OF SNP rs2267735

| | U.S. Population - 11,676 female genome sequences among 17,131 genome sequences | | | |
|---|---|---|---|---|
| | Caucasians | Caucasians | African- | Asian- |
| | (White) | (Hispanic) | Americans | Americans |
| C/G | 47.92%/52.08% | 46.77%/53.23% | 66.24%/33.76% | 46.12%/53.88% |
| | Presumptive ‘Ancestral’ Genome Sequences from 1000 genomes project | | | |
| | “averaged” | | “averaged” | “averaged” |
| | CEU + TSI | MEX | YRI + MKK + ASW | JPT + CHB + CHD |
| C/G | 45.8%/54.2% | 43.0%/57.0% | 64.0%/36.0% | 48.93%/48.93% |

CEU: Utah residents with Northern and Western European ancestry; TSI: Toscans in Italy
MEX: Mexican ancestry in Los Angeles, California
YRI: Yoruba in Ibadan, Nigeria; MKK: Maasai in Kinyawa, Kenya; ASW: African ancestry in Southwest USA
JPT: Japanese in Tokyo, Japan; CHB: Han Chinese in Beijing, China; CHD: Chinese in Metropolitan Denver, Colorado

The REF SEQ ID (GRCh37.p5) is incorporated herein by reference.

TABLE 9
Novel SNP in ADCYAP1R1 exons that may impact
drug response.

| | | | | SEQ |
|---|---|---|---|---|
| | | | | ID |
| | SNP | Position | MAF | NO: |
| A | CGCTTGCTAAT A/C | chr7: 31,104,185 | 4% | 16 |
| | TTATTATAAGAT | | | |

ADRA2A

This is one of the alpha-2-adrenergic receptors, members of the G protein-coupled receptor superfamily. The family includes 3 highly homologous subtypes: alpha2A, alpha2B, and alpha2C. These receptors have a critical role in regulating neurotransmitter release from sympathetic nerves and from adrenergic neurons in the central nervous system. Studies in mouse revealed that both the alpha2A and alpha2C subtypes were required for normal presynaptic control of transmitter release from sympathetic nerves in the heart and from central noradrenergic neurons; the alpha2A subtype inhibited transmitter release at high stimulation frequencies, whereas the alpha2C subtype modulated neurotransmission at lower levels of nerve activity. This gene encodes alpha2A subtype, and it contains no introns in either its coding or untranslated sequences. ADRA2A is a small gene with a sequence length of <4000 bp. The rank order of potency for agonists of this receptor is oxymetazoline>clonidine>epinephrine>norepinephrine>phenylephrine>dopamine>p-synephrine>p-tyramine>serotonin=p-octopamine. For antagonists, the rank order is yohimbine>phentolamine=mianserine>chlorpromazine=spiperone=prazosin>propanolol>alprenolol=pindolol.

ADRA2A polymorphisms and pharmacogenomics: Metabolic syndrome in patients taking antipsychotic medications: Previous studies found an association between the 1291C/G polymorphism (rs1800544) in the promoter region of the ADRA2A gene and clozapine- or olanzapine-induced weight gain. In both studies, in Asians, the G allele was associated with increased weight gain expressed as a >7% (Wang et al.; 8.45 kg vs 2.79 kg; p=0.023) or 10% (odds ratio [OR]:2.58 [95% CI 1-1.21-5.51]) increase in body weight. In contrast, another study showed that an association in the opposite direction was found for Caucasians. Caucasian patients carrying the C allele experienced more weight gain than patients with the G/G genotype (3.73 kg vs 0.23 kg; p=0.013), demonstrating the potential impact of ethnicity on the association. These results are consistent with the instant data and those of the 1000 Genomes Project in Table 10.

TABLE 10
ALLELE FREQUENCY OF SNP rs1800544

| | U.S. Population -17,131 genome sequences | | | |
|---|---|---|---|---|
| | Caucasians | Caucasians | African- | Asian- |
| | (White) | (Hispanic) | Americans | Americans |
| C/G | 69.88%/30.12% | 53.89%/46.11% | 19.67%/80.33% | 32.45%/67.55% |
| | Presumptive ‘Ancestral’ Genome Sequences from 1000 genomes project | | | |
| | “averaged” | | | “averaged” |
| | CEU + TSI | MEX | YRI | JPT + CHB |
| C/G | 72.50%/27.50% | 53.0%/47.0% | 23.7%/76.3% | 27.50%/72.50% |

CEU: Utah residents with Northern and Western European ancestry
MEX: Mexican ancestry in Los Angeles, California
YRI: Yoruba in Ibadan, Nigeria
JPT: Japanese in Tokyo, Japan; CHB: Han Chinese in Beijing, China

Attention deficit hyperactivity disorder (ADHD) and ADRA2A polymorphisms: SNP association studies have found no significant association between rs1800544 or rs553668 and ADHD, either in children or adults (see de Cerqueira, C. C. S., et al. Psychiatry Res. (2010) ADRA2A polymorphisms and ADHD in adults: Possible mediating effect of personality, incorporated herein by reference). Instead, a more complex picture is emerging, suggesting that, in adults with personality trait components of ADHD, including novelty seeking, harm avoidance and persistence, there is a highly significant correlation between the haplotype block that contains rs1800544 and rs553668 and ADHD.

The REF SEQ ID (GRCh37.p5) is incorporated herein by reference.

TABLE 11
Novel SNPs in ADRA2A pharmaocogene exons
that may impact drug response.

| | | | | SEQ |
|---|---|---|---|---|
| | | | | ID |
| | SNP | Position | MAF | NO: |
| A | GAGCGCGGGC | chr10: 112,838,563 | 0.6% | 17 |
| | C/G CGAGCG | | | |
| B | AGCGCAGCGC | chr10: 112,838,576 | 2% | 18 |
| | G/C GGCCCC | | | |

Brain Derived Neurotropic Factor (BDNF)

The protein encoded by this gene is a member of the nerve growth factor family. It is induced by cortical neurons and is necessary for survival of striatal neurons in the brain. Expression of this gene is reduced in both Alzheimer's and Huntington disease patients. This gene may play a role in the regulation of stress response and in the biology of mood disorders. Multiple transcript variants encoding distinct isoforms have been described for this gene. In humans, the gene is located on chromosome 11, from 27,676,440 to 27,743,605 reverse strand, spanning 67,165 nucleotides. The gene produces up to 18 transcripts through alternative splicing mechanisms, in a tissue-specific manner. There is also BDNF-AS1 gene (antisense RNA 1; non-protein coding) that may play a role in the regulation of transcription at the mRNA level.

BDNF acts as a signal for proper axonal growth and when secreted from target tissues, it binds to TrkB receptors and is internalized to signal in the nucleus to stimulate neurite outgrowth. BDNF is known to be required for proper development and survival of dopaminergic, GABAergic, cholinergic, and serotonergic neurons. BDNF also serves essential functions in the mature brain in synaptic plasticity and is crucial for learning and memory. BDNF and TrkB are co-localized at pre- and postsynaptic sites, where BDNF can be released in an activity-dependent manner. Presynaptic BDNF signaling promotes neurotransmitter release, whereas postsynaptic BDNF signaling is involved in enhancing various ion channel function including the a-amino-3-hydroxy-5-methyl-4-isoxazolepropionic acid receptor, the NMDA receptor, transient receptor potential cation channels, as well as sodium and potassium channels. BDNF acts at both excitatory and inhibitory synapses, and experimental evidence suggests that BDNF may modulate both spontaneous and stimulated neuronal activity.

Further studies of loss of BDNF signaling in the adult brain have led to the discovery of many more roles for BDNF in the modulation of behavior. In addition to its importance in learning, other studies have shown that BDNF plays an important role in cognition as well as mood-related behaviors. For this reason, BDNF is widely studied in relation to neuropsychiatric diseases, including but not limited to major depressive disorder, schizophrenia, bipolar disorder, addiction, Rett syndrome, and eating disorders.

BDNF polymorphisms and pharmacogenomics: Major depressive disorder (MDD): Researchers have examined the BDNF gene for SNPs that may be linked to MDD. One of the most common BDNF SNPs, rs6265, in humans is located at codon 66, resulting in a Val to Met (V66M) protein variant, which prevents the activity-dependent release of BDNF. Although this polymorphism does seem to affect human cognition, the contribution of this mutation to the pathological features of MDD or to suicidality still remains unclear. Recent studies have revealed that men homozygous for the mutation may be at greater risk for MDD, and this SNP may increase susceptibility for MDD after early-life stress.

Eating disorders: Variations in BDNF are associated with susceptibility to bulimia nervosa (BN). Several genes with an essential role in the regulation of eating behavior and body weight are considered candidates involved in the etiology of eating disorders, but no relevant susceptibility genes with a major effect on anorexia nervosa or bulimia nervosa have been identified. BDNF has been implicated in the regulation of food intake and body weight in rodents. A strong association between the rs6265 BDNF variant and restricting and low minimum body mass index in Spanish patients has been reported. Another single nucleotide polymorphism located in the promoter region of the BDNF gene had an effect on BN and late age at onset of weight loss. These are two variants associated with the pathophysiology of eating disorders (ED) in different populations. These variants support a role for BDNF in the susceptibility to aberrant eating behaviors.

Antipsychotic drug response in schizophrenia: Three functional genetic polymorphisms in BDNF are associated with risperidone response in schizophrenic Chinese patients from Shanghai. The frequency of the 230-bp allele of the (GT)n dinucleotide repeat polymorphism was much higher in responders than in risperidone non-responders and that the difference was statistically significant even after Bonferroni's adjustment for multiple testing. It was also found that two haplotypes constructed with the three polymorphisms were significantly related to the response to risperidone, which implied that patients with the 230-bp allele of the (GT)n dinucleotide repeat polymorphism or the 230-bp/C-270/rs6265G haplotype had a better response to risperidone than those with other alleles or haplotypes (especially those with the 234-bp allele and the 234-bp/C-270/rs6265A haplotype). These findings are consistent with the roles of 230 and 234-bp alleles of the (GT)n dinucleotide repeat polymorphism in the therapeutic response to risperidone, which indicates that the effects of haplotypes were mainly driven by the (GT)n dinucleotide repeat polymorphism and that genotyping of the dinucleotide repeat polymorphism is sufficient to assess the major influence of BDNF on response. The 230-bp allele and the 170-bp allele contain the same number of dinucleotide repeats. The studies indicated that a lower number of dinucleotide repeats was associated with a better response to antipsychotics.

Epistasis: BDNF SNPs have been shown to have synergistically interact with other genes and SNPs (e.g., an interaction between rs6265 and CRHR1SNPs).

The REF SEQ ID (GRCh37.p5) is incorporated herein by reference.

TABLE 12
Novel SNPs in BDNF pharmacogene exons
that may impact drug response.

| | | | | SEQ |
|---|---|---|---|---|
| | | | | ID |
| | SNP | Position | MAF | NO: |
| A | GAAGTCCT | chr11: 27,699,480 | 1% | 19 |
| | G/C GGGT | | | |
| B | ATT T/A | chr11: 27,699,475 | 3% | 20 |
| | TTTACCAAC | | | |

Catechol-O-methyltransferase

Catechol-O-methyltransferase is one of several enzymes that degrade catecholamines, such as dopamine, epinephrine, and norepinephrine. In humans, the catechol-O-methyltransferase protein is encoded by the COMT gene. The regulation of catecholamines is impaired in a number of medical conditions. Several pharmaceutical drugs target COMT to alter its activity and therefore the availability of catecholamines.

The COMT protein is encoded by the gene COMT spanning chromosome 22 from 19,929,263-19,957,498. The gene is associated with allelic variants. COMT degrades catecholamines, including dopamine. Two main COMT protein isoforms are known. In most assayed tissues, a soluble cytoplasmic (S-COMT consisting of 4 exons) isoform predominates. In the brain, a longer membrane-bound form (MB-COMT consisting of 6 exons) is the major species. Although expressed widely, COMT appears to be a minor player in dopamine clearance compared with neuronal synaptic uptake by the dopamine transporter and subsequent monoamine oxidase (MAO) metabolism. However, in the prefrontal cortex (PFC) where dopamine transporter expression is low, the importance of COMT appears to be greater.

The structure of the COMT gene, which lies on chromosome 22q11, produces two major transcripts. A number of putative regulatory elements have been discovered in the COMT gene, which may explain the differential expression of the long and short transcripts in different tissues. These include numerous estrogen response elements, and estradiol has been shown to down-regulate COMT expression in cell culture. A recent report suggests that MB-COMT exists in two forms which may be differentially affected by the Val/Met genotype. Thus, there may be a level of genetic complexity including possible gender-specific effects.

COMT polymorphisms: A common G>A polymorphism is present in COMT that produces a valine-to-methionine (Val/Met) substitution at codons 108 and 158 of S-COMT and MB-COMT, respectively, that results in a trimodal distribution of COMT activity in human populations. The polymorphism is usually referred to as the Val/Met locus, but is also known by the reference sequence identification code rs4680 (previously rs165688). Terminology varies: the Valine (Val) allele is also referred to as the high activity (H) allele or the G allele. Polymorphism and haplotype frequencies at COMT have been shown to vary substantially across populations. For example, the Val allele has been reported at frequencies varying between 0.99 and 0.48.14 Moreover, in certain Asian populations, a second functional variant, Ala72Ser, (MB COMT nomenclature) has been reported. Hence, population origin of samples is a potentially important variable for interpreting genetic studies of COMT.

In terms of many studies showing association of the rs4680 to a variety of psychiatric diseases, including Panic Disorder, OCD, ADHD, Bipolar Disorder and Schizoaffective disorder, the best evidence suggests that it plays a major role in the etiology of Schizophrenia. Other strong associations include adenomyosis endometriosis, aggressive personality traits, alcoholism, anorexia nervosa, breast cancer, cognitive function, eating disorders, estradiol, sex hormone binding globulin, heroin abuse, hormone disturbance, hypertension, information processing, menarche, menopause, neuroticism, ovarian cancer, oxidative stress, Parkinson's disease, performance on the Wisconsin Card Sorting Test, prostate carcinoma, smoking cessation, and suicide.

From the bulk of the literature, the following conclusions can be drawn:

A strong body of data supports an effect of the COMT SNP rs4680 (Val/Met) locus on frontal lobe function (Val associated with poorer function).

Both positional and functional evidence makes the COMT gene a strong a priori candidate for involvement in psychosis and other psychiatric phenotypes.

There has been substantial study of schizophrenia and to a lesser extent, bipolar disorder, at least for the rs4680 polymorphism.

A single, simple main effect of rs4680 can be excluded for schizophrenia and bipolar disorder.

Positive findings from studies of multiple polymorphisms are promising and appear to be more common than expected by chance alone.

Despite more extensive study, the genetic evidence for the involvement of COMT in psychosis is less compelling than for dysbindin, neuregulin 1, DISC1 or DAOA.

The optimal clinical phenotype definition for studies of COMT is not yet known

Phenotypes other than schizophrenia and bipolar disorder have yet to be studied in large samples.

For all phenotypes, there is a requirement for more studies, larger samples and systematic analysis of variation across the gene.

As a consequence of both its chromosomal location in a region of interest for psychosis and mood disorders and its function as an enzyme involved in catabolism of monoamines, COMT has been one of the most studied genes for psychosis. On the basis of prior probabilities, it would seem surprising if variation at COMT did not have some influence either on susceptibility to psychiatric phenotypes, modification of the course of illness, or moderation of response to treatment. There is now robust evidence that variation at COMT influences frontal lobe function. However, despite considerable research effort, it has not proven straightforward to demonstrate and characterize a clear relationship between genetic variation at COMT and psychiatric phenotypes.

The REF SEQ ID (GRCh37.p5) is incorporated herein by reference.

TABLE 13
Novel SNPs in COMT pharmacogene exons
that may impact drug response.

| | | | | SEQ |
|---|---|---|---|---|
| | | | | ID |
| | SNP | Position | MAF | NO: |
| A | GACCCGATC C/A | chr22: 19,929,220 | 0.5% | 21 |
| | TACACCTGCT | | | |
| B | CTGCGCCGGACCG G/T | chr22: 19,929,384 | 3% | 22 |
| | GGCGGGT | | | |
| C | TCGGGGCGGG G/C | chr22: 19,929,460 | 4% | 23 |
| | GCCTTCA | | | |

CRHBP (Corticotropin-Releasing Hormone Binding Protein)

The CRHBP protein is a potent stimulator of synthesis and secretion of preopiomelanocortin-derived peptides. Although corticotropin-releasing hormone (CRH) concentrations in the human peripheral circulation are normally low, they increase throughout pregnancy and fall rapidly after parturition. Maternal plasma CRH probably originates from the placenta. Human plasma contains a CRH-binding protein which inactivates CRH and which may prevent inappropriate pituitary-adrenal stimulation in pregnancy.

The human CRHBP gene has been cloned and mapped to the distal region of chromosome 13. The gene consists of 7 exons and 6 introns. The mature protein has 10 cysteines and 5 tandem disulfide bridges, 4 of which are contained within exons 3, 5, 6, and 7. One bridge is shared by exons 3 and 4. The signal peptide and the first 3 amino acids of the mature protein were encoded by an extreme 5′ exon. Primer extension analyses revealed the transcriptional initiation site to be located 32 bp downstream from a consensus TATA box. The promoter sequence contained a number of putative promoter elements, including an AP-1 site, three ER-half sites, the immunoglobulin enhancer elements NF-kappa B and INF-1, and the liver-specific enhancers LFA1 and LFB1.

CRHBP polymorphisms, suicide, and anti-depressant drug response: A SNP in the CRHBP gene, rs10473984, is located at the 3′ end of the gene, and is highly associated with suicidal behavior in patients with schizophrenia. The T allele, associated with poorer response to citalopram treatment, was also associated with higher corticotropin serum concentrations in depressed and non-depressed individuals. This suggests that this allele is associated with reduced CRHBP expression and thus higher levels of free CRH, thereby increasing corticotropin secretion. In addition, individuals with clinically significant depressive symptoms carrying the GG genotype (associated with best treatment outcome) of this SNP showed the least degree of dexamethasone suppression of corticotropin. Previous studies have shown that depressed patients with dexamethasone non-suppression of HPA-axis activation at treatment initiation have a beneficial treatment-response profile.

Results to date support the role of the CHRBP SNP rs10473984 and the CRF system in treatment response to citalopram in patients with MDD. Results to date expand upon previous preclinical and clinical studies that demonstrated a central role of this system in the pathophysiology of depression and mechanism of action of antidepressants. Results support the notion that genetic variants in components of the CRH system might be most relevant in predicting treatment response in anxious depression.

The REF SEQ ID (GRCh37.p5) is incorporated herein by reference.

TABLE 14
Novel SNPs in CRHBP pharmacogene exons
that may impact drug response.

| | | | | SEQ |
|---|---|---|---|---|
| | | | | ID |
| | SNP | Position | MAF | NO: |
| A | CTCAG T/C | chr5: 76,257,015 | 2% | 24 |
| | TTCTGCCATTG | | | |

CRHR1 (Corticotropin Releasing Hormone Receptor 1)

The CRHR1 gene encodes a G-protein coupled receptor that binds neuropeptides of the corticotropin releasing hormone family that are major regulators of the hypothalamic-pituitary-adrenal pathway. The encoded protein is essential for the activation of signal transduction pathways that regulate diverse physiological processes including stress, reproduction, immune response and obesity. Alternative splicing results in multiple transcript variants, one of which represents a read-through transcript with the neighboring gene MGC57346. CRHR1 is an important mediator in the stress response. Cells in the anterior lobe of the pituitary gland known as corticotropes express CRHR1 receptors and will secrete adrenocorticotropic hormone (ACTH) when stimulated. CRHR1 receptors are abundantly expressed in the CNS with major expression in the cortex, cerebellum, hippocampus, amygdala, olfactory bulb and pituitary. In the periphery, CRHR1 receptors are expressed at low levels in the skin, ovary, testis and adrenal gland. CRHR1 receptors regulate ACTH release and the stress response. The human gene encoding the CRHR1 receptor is localized on chromosome 17 (17q12-q22).

CRHR1 polymorphisms: Variations in the CRHR1 gene are associated with enhanced response to inhaled corticosteroid therapy in asthma. CRHR1 receptor antagonists are being actively studied as possible treatments for depression and anxiety. The risk of suicide, which causes about 1 million deaths each year, is considered to augment as the levels of stress increase. Dysregulation in the stress response of the hypothalamic-pituitary-adrenocortical (HPA) axis, involving the corticotrophin-releasing hormone (CRH) and its main receptor (CRHR1), is associated with depression, frequent among suicidal males. There is a highly reproducible association between a SNP in the CRHR1 gene (rs4792887) with people exposed to low levels of stress who attempt suicide. Results from healthy controls and a preliminary sample of MDD participants show that the CRHR1SNP rs110402 moderates neural responses to emotional stimuli, suggesting a potential mechanism of vulnerability useful for the development of MDD. In addition, studies of gene X gene and gene X environment interactions show that CRHR1 SNPs are significantly associated with polymorphisms in the CHRBP, FKBP05 and SLC6A4 genes. CRHR1 polymorphisms have also been associated with binge-drinking in several studies (See, e.g., Treutline et al. Molecular Psychiatry, 11:594-602, 2006).

The REF SEQ ID (GRCh37.p5) is incorporated herein by reference.

TABLE 15
Novel SNPs in CRHR1 pharmacogene exons
that may impact drug response.

| | | | | SEQ |
|---|---|---|---|---|
| | | | | ID |
| | SNP | Position | MAF | NO: |
| A | GGCCAGGC A/T | chr17: 43,887,382 | 1% | 25 |
| | CGTGGCT | | | |
| B | CGGGCTTG G/C/T | chr17: 43,887,520 | 0.2% | 26 |
| | TGGTG | | | |
| C | CCGGGCTT G/C | chr17: 43,887,514 | 0.8% | 27 |
| | GTGGTGG | | | |
| D | CCCA G/T | chr17: 43,887,397 | 2% | 28 |
| | CGCTTTGGGAGG | | | |

DBI (Diazepam Binding Inhibitor Protein)

The DBI gene encodes diazepam binding inhibitor (DBI), a protein that is regulated by hormones and is involved in lipid metabolism and the displacement of betacarbolines and benzodiazepines, which modulate signal transduction at type α gamma-aminobutyric acid receptors located at post-synaptic sites in the brain. The protein is conserved from yeast to mammals, with the most highly conserved domain consisting of seven contiguous residues that constitute the hydrophobic binding site for medium- and long-chain acyl-Coenzyme A esters. Diazepam binding inhibitor also mediates the feedback regulation of pancreatic secretion and the postprandial release of cholecystokinin, in addition to its role as a mediator in corticotropin-dependent synthesis of steroids in the adrenal gland. Three pseudogenes located on chromosomes 6, 8 and 16 have been identified. Multiple transcript variants encoding different isoforms have also been described for this gene.

Diazepam-binding inhibitor (DBI) is a highly conserved 10 kD polypeptide expressed in various organs and implicated in the regulation of multiple biological processes such as GABAα/benzodiazepine receptor modulation, acyl-CoA metabolism, steroidogenesis, and insulin secretion. The gene is differentially regulated by androgen, including multiple transcripts originating from multiple transcription start sites and alternative processing. The most abundant type of transcripts (referred to as type 1 transcripts) encode a DBI protein of 86 amino acids, while the minor type (type 2 transcripts) harbors an insertion of 86 bases and might encode an unrelated protein of 67 amino acids. Examination of a cloned DBI gene revealed a structural organization of four exons present in all transcripts and one alternatively used exon present only in type 2 transcripts. The promoter region is located in a CpG island and lacks a canonical TATA box. Transient transfection of DBI promoter fragments into transfected cells demonstrated that a 1.1 kb region upstream of the translation start site is able to drive high-level expression of luciferase in transfected cells in an androgen-regulated fashion. Taken together these data indicate that the isolated human gene encoding DBI is functional, has a high degree of structural similarity with the corresponding rat gene, exhibits hallmarks of a typical housekeeping gene, and harbors cis-acting elements that are at least partially responsible for androgen-regulated transcription.

The REF SEQ ID (GRCh37.p5) is incorporated herein by reference.

TABLE 16
Novel SNPs in DBI pharmacogene exons
that may impact drug response.

| | | | | SEQ |
|---|---|---|---|---|
| | | | | ID |
| | SNP | Position | MAF | NO: |
| A | CAGG A/T ACCACATTT | chr2: 120,127,424 | 0.7% | 29 |
| B | CATTTCA G/C GTACTT | chr2: 120,127,455 | 3% | 30 |
| C | TGTGGCAA G/T TGGCT | chr2: 120,127,471 | 0.2% | 31 |
| D | ATTGGA C/G AATTGC | chr2: 120,127,490 | 5% | 32 |
| E | TACATTT C/T CATTTC | chr2: 120,127,513 | 4% | 33 |
| F | TCCA C/G CGCTTGGAG | chr2: 120,127,521 | 3% | 34 |
| G | GCAGTTT G/C TTTCAG | chr2: 120,127,587 | 0.8% | 35 |
| H | AAGCGC T/A CAGGGAC | chr2: 120,127,624 | 2% | 36 |
| I | CCAACTGCA G/C ATGA | chr2: 120,127,750 | 0.4% | 37 |
| J | TTCACGG G/C CAAGGC | chr2: 120,128,343 | 1% | 38 |
| K | AAGTGGG A/C TGCCTG | chr2: 120,128,358 | 0.3% | 39 |
| L | GCCTGG A/G ATGAGCT | chr2: 120,128,366 | 4% | 40 |
| M | TGGAATG A/T GCTGAA | chr2: 120,128,370 | 0.7% | 41 |
| N | TAAATA A/G AAGAATC | chr2: 120,127,397 | 2% | 42 |
| O | AAATAG T/A TAAATAA | chr2: 120,127,390 | 5% | 43 |
| P | TTAGTCT T/C CATTCAC | chr2: 120,127,413 | 4% | 44 |
| Q | ATCAA G/C TTAGTCTTC | chr2: 120,127,403 | 2% | 45 |
| R | GATGCCT G/AGAATGAG | chr2: 120,128,364 | 1% | 46 |

DRD2 (Dopamine Receptor Type 2)

The DRD2 gene encodes the D2 subtype of the dopamine receptor. This G-protein coupled receptor inhibits adenylyl cyclase activity. A missense mutation in this gene causes myoclonus dystonia; other mutations have been associated with schizophrenia. Alternative splicing of this gene results in two transcript variants encoding different isoforms. A third variant has been described, but it has not been determined whether this third form is normal or due to aberrant splicing. D2 receptors are members of the dopamine receptor G-protein-coupled receptor family that also includes D1, D3, D4 and D5. They are located primarily in the caudate putamen, nucleus accumbens and olfactory tubercle where they are involved in the modulation of locomotion, reward, reinforcement and memory and learning. The human D2 receptor gene has been localized to chromosome 11 (11q22-23).

DRD2 polymorphisms: The D2 dopamine receptor (DRD2) has been one of the most extensively investigated gene in neuropsychiatric disorders. After the first association of the TaqI A DRD2 minor (A1) allele with severe alcoholism in 1990, a large number of international studies have followed. A meta-analysis of these studies of Caucasians showed a significantly higher DRD2 A1 allelic frequency and prevalence in alcoholics when compared to controls. Variants of the DRD2 gene have also been associated with other addictive disorders including cocaine, nicotine and opioid dependence and obesity. It is hypothesized that the DRD2 is a reinforcement or reward gene. The DRD2 gene has also been implicated in schizophrenia, posttraumatic stress disorder, movement disorders and migraine. Phenotypic differences have been associated with DRD2 variants. These include reduced D2 dopamine receptor numbers and diminished glucose metabolism in brains of subjects who carry the DRD2 A1 allele. In addition, pleiotropic effects of DRD2 variants have been observed in neurophysiologic, neuropsychologic, stress response, personality and treatment outcome characteristics.

Three polymorphisms in DRD2 have received the greatest attention. These include the Taq1A polymorphism, which is located approximately 10 kb from the 3′ end of the gene and has no known functional effect; the −141-C Ins/Del polymorphism in the promoter region, which has been associated with lower expression of the D2 receptor in vitro (487) and higher D2 density in the striatum in vivo; and Ser311Cys, a relatively common coding polymorphism that has been shown to reduce signal transduction via the receptor. At least fourteen studies have examined the relationship between DRD2 polymorphisms and efficacy of both FGAs and SGAs, while twenty-one studies have investigated adverse effects, including TD, weight gain and neuromalignant syndrome. In a recent meta-analysis of four different genes and TD, a significant association was found with the Taq1A polymorphism in DRD2.

Many antipsychotic medications carry a substantial liability for weight gain, and one mechanism common to all antipsychotics is binding to the DRD2 receptor. Examination of the relationship between −141C Ins/Del (rs1799732) (a functional promoter region polymorphism in DRD2), and antipsychotic-induced weight gain, in deletion allele carriers shows significantly more weight gain after 6 weeks of treatment regardless of assigned medication. Although deletion carriers were prescribed higher doses of olanzapine (but not risperidone), dose did not seem to account for the genotype effects on weight gain. It is possible that DRD2 promoter region variation may render D2 receptors differentially sensitive to the effects of antipsychotic medications on reward signals associated with food intake and satiety.

The REF SEQ ID (GRCh37.p5) is incorporated herein by reference.

TABLE 17
Novel SNPs in DRD2 pharmacogene exons
that may impact drug response.

| | | | | SEQ |
|---|---|---|---|---|
| | | | | ID |
| | SNP | Position | MAF | NO: |
| A | GCTGAGCT A/T | chr11: 113,313,103 | 1% | 47 |
| | CAAAGGCT | | | |
| B | GCTGTG T/A | chr11: 113,313,127 | 0.5% | 48 |
| | CTGAATGATG | | | |
| C | CTCAGAT C/G | chr11: 113,313,147 | 3% | 49 |
| | CTCTCACCTA | | | |
| D | AGGAGGA G/T | chr11: 113,313,189 | 0.2% | 50 |
| | GAGCACTCTT | | | |
| E | GTTGATTTT C/G | chr11: 113,313,256 | 5% | 51 |
| | TCACCTCC | | | |

DRD4 (Dopamine Receptor Type 4)

The DRD4 gene encodes the D4 subtype of the dopamine receptor. The D4 subtype is a G-protein coupled receptor which inhibits adenylyl cyclase. It is a target for drugs which treat schizophrenia and Parkinson disease. Mutations in this gene have been associated with various behavioral phenotypes, including autonomic nervous system dysfunction, attention deficit/hyperactivity disorder, and the personality trait of novelty seeking. This gene contains a polymorphic number (2-10 copies) of tandem 48 nucleotide repeats; the sequence shown contains four repeats. DRD4 has been examined as a gene of interest for behavioral and psychiatric phenotypes in part because of its genetic variability. The DRD4 gene contains a 48-base pair variable number of tandem repeats (VNTR) in exon III with lengths varying from two to 11 repeats, three with common variant of 2(D4.2), 4 (D4.4) and 7 repeats (D4.7). Variations in length of the VNTR have been shown to have functional effects on the receptor. In vitro, while the D4.7 variant does not appear to bind dopamine antagonists and agonists with greater affinity than the D4.2 or D4.4 variants. D4 receptors are structurally very similar to D2 receptors and are localized in various brain regions, including the cerebral cortex, amygdala, hypothalamus, the pituitary and other limbic brain structures. Expression of D4 receptors in the prefrontal cortex is of particular interest for behavioral phenotypes as these regions are involved in attention and cognition. DRD4 VNTR variation has been associated with a wide array of behavioral tendencies and psychiatric conditions. Among the most consistent are the association between 7R+ and ADHD and the finding that 7R+ individuals exhibit augmented anticipatory desire response to stimuli signaling dopaminergic incentives, such as food, alcohol, tobacco, gambling, sexual promiscuity and progressive beliefs.

The REF SEQ ID (GRCh37.p5) is incorporated herein by reference.

TABLE 18
Novel SNPs in DRD4 pharmacogene exons
that may impact drug response.

| | | | | SEQ |
|---|---|---|---|---|
| | | | | ID |
| | SNP | Position | MAF | NO: |
| A | ATTCGGG G/C GAGCTGAGGC | chr11: 638,979 | 0.4% | 52 |
| B | CGGAGGTTGC A/G GTGAGTT | chr11: 639,023 | 5% | 53 |
| C | AGACTGA G/C GTGGGAGGAT | chr11: 639,157 | 0.8% | 54 |

FK06 Binding Protein 51 (FKBP5)

FKBP5 is a 51 kDa protein encoded by a gene on the short arm of human chromosome 6 (6p21.31) in the human. It regulates glucocorticoid receptor (GR) sensitivity. When it is bound to the receptor complex, cortisol binds with lower affinity and nuclear translocation of the receptor is less efficient. FKBP5 mRNA and protein expression are induced by GR activation via intronic hormone response elements and this provides an ultra-short feedback loop for GR-sensitivity. The protein encoded by this gene is a member of the immunophilin protein family, which plays a role in immunoregulation and basic cellular processes involving protein folding and trafficking. This encoded protein is a cis-trans prolyl isomerase that binds to the immunosuppressants FK506 and rapamycin. FKBP5 is thought to mediate calcineurin inhibition. FKBP5 also interacts functionally with mature hetero-oligomeric progesterone receptor complexes along with the 90 kDa heat shock protein and P23 protein. The gene FKBP5 has been found to have multiple polyadenylation sites. Alternative splicing results in multiple transcript variants.

FKBP5 pharmacogenomics: Polymorphisms in the gene encoding this co-chaperone have been shown to be correlated with differential upregulation of FKBP5 following GR activation and differences in GR sensitivity and stress hormone system regulation. Alleles associated with enhanced expression of FKBP5 following GR activation lead to an increased GR resistance and decreased efficiency of the negative feedback of the stress hormone axis in healthy controls. This results in a prolongation of stress hormone system activation following exposure to stress. This dysregulated stress response might be a risk factor for stress-related psychiatric disorders. In fact, these same alleles are over-represented in individuals with major depression, bipolar disorder and post-traumatic stress disorder. In addition, these alleles are also associated with faster response to antidepressant treatment. Thus, FKBP5 is a potential therapeutic target for the prevention and treatment of stress-related psychiatric disorders.

Data from PharmGkb.org is shown in Table 19:

| | TYPE/ | STRENGTH OF | | |
|---|---|---|---|---|
| SNP | EFFECT | EVIDENCE* | DRUG | DISEASE |
| rs3800373 | Efficacy | 2 | antidepressants | Depression |
| rs1360780 | Efficacy | 2 | antidepressants | Depression |

FKBP5 and antidepressant drug response: Several FKBP5 polymorphisms are associated with differential response to antidepressant drugs. There have been multiple studies in Caucasians, Asians, and other ethnicities of an association between polymorphisms in FKBP5 and response to antidepressant drugs in 280 depressed patients of the MARS sample as well as a small independent German replication sample. Patients homozygous for the high-induction alleles responded over 10 days faster to antidepressant treatment than patients with the other two genotypes. This effect appears independent of the class of antidepressant drug, as it was observed in groups of patients treated with either tricyclic antidepressants, selective serotonin reuptake inhibitor or mirtazapine. This suggests that the mechanisms by which FKBP5 is involved in treatment response are downstream of the primary binding profile of antidepressant drugs. This finding has now been supported in two further studies, the STAR*D cohort as well as an additional German sample. The odd ratios (ORs) in these replication studies were much smaller than the ones reported initially—about 5.0 to 23.0 reported initially—and ranged from about 1.3 to 1.8, much more within the expectations for more complex genetic phenotypes. Two smaller studies, with Spanish and Korean ethnic groups, have reported negative associations. The differences in ORs could indicate either an over-estimation of the effect size in the initial sample (also termed “winners curse”) or an actual difference in the samples (such as ethnicity or disease sub-types). In addition, in the absence of placebo controlled data, it cannot be excluded that the observed association between the high-induction FKBP5 polymorphisms and response to antidepressant is in fact a pharmacogenetic effect or related to an inherently different duration of depressive episodes in these patients.

As described above, the high-induction alleles of FKBP5 that are associated with GR resistance in healthy controls are associated with enhanced GR-sensitivity in depressed patients as compared to patients carrying the other alleles. In fact, in the patients carrying the genotypes associated with faster response to antidepressant treatment, HPA-axis hyper-activity as measured by the Dex—CRH test at in-patient admission was significantly reduced compared to the other patients. This might have facilitated the normalization of HPA-axis hyperactivity that is associated with clinical response to most antidepressant treatments.

FKBP5 and PTSD: There are many studies showing that FKBP5 SNPs are strongly associated with posttraumatic stress disorder, and can even be used to define subtypes of the disorder. The FKBP5 SNP rs9296158 genotype increases the risk for PTSD with early trauma. Also, rs9296158 may be used to identify biologically different subtypes of PTSD in that the genotype groups differed with respect to PTSD-related changes in GR sensitivity. This was reflected in genotype- and PTSD-dependent differences in the expression of GR-dependent transcripts in whole blood.

The REF SEQ ID (GRCh37.p5) is incorporated herein by reference.

TABLE 20
Novel SNPs in FKBP5 pharmacogene exons that may impact drug response.

| | | | | SEQ ID |
|---|---|---|---|---|
| | SNP | Position | MAF | NO: |
| A | TGTCCTATTT T/A TGAATGG | chr6: 35,598,999 | 2% | 55 |
| B | ATGGTGAA C/G AAACTGTGG | chr6: 35,599,011 | 0.5% | 56 |
| C | AAATTGT G/C GAATACTTCT | chr6: 35,599,034 | 0.3% | 57 |
| D | AGGAATTC A/T ACATGCATG | chr6: 35,599,054 | 4% | 58 |
| E | GTCAACACC A/G AAGATAAT | chr6: 35,599,104 | 1% | 59 |
| F | AGGCAAAA T/A TATAGTAAA | chr6: 35,599,152 | 3% | 60 |
| G | TATAGTAA C/T AGAAACCAA | chr6: 35,599,161 | 0.4% | 61 |
| H | ATAAAATA C/G TTTTTAGGG | chr6: 35,599,235 | 2 | 62 |
| I | TTTATTATA C/G GTAAATAA | chr6: 35,599,341 | 0.8% | 63 |
| J | AATTCATC A/T AACTATATAC | chr6: 35,599,307 | 3% | 64 |

GCR(NR3C1)

The glucocorticoid receptor (GR, or GCR) also known as NR3C1 (nuclear receptor subfamily 3, group C, member 1) is the receptor to which cortisol and other glucocorticoids bind. The GR is expressed in almost every cell in the body and regulates genes controlling development, metabolism, and immune response. Because the receptor gene is expressed in several forms, it has many different (pleiotropic) effects in different parts of the body. When the GR binds to glucorticoids, its primary mechanism of action is the regulation of gene transcription. The unbound receptor resides in the cytosol of the cell (the part of the cell outside of the nucleus). After the receptor is bound to glucocorticoid, the receptor-glucorticoid complex can take either of two paths. The activated GR complex up-regulates the expression of anti-inflammatory proteins in the nucleus or represses the expression of pro-inflammatory proteins in the cytosol (by preventing the translocation of other transcription factors from the cytosol into the nucleus). In humans, the GR protein is encoded by NR3C1 gene, which is located on chromosome 5 (501) and spans 126,549 bases.

In the absence of hormone, the glucocorticoid receptor (GR) resides in the cytosol complexed with a variety of proteins, including heat shock protein 90 (hsp90), the heat shock protein 70 (hsp70) and the protein FKBP52 (FK506-binding protein 52). The endogenous glucocorticoid hormone cortisol diffuses through the cell membrane into the cytoplasm and binds to the glucocorticoid receptor (GR) resulting in release of the heat shock proteins. The resulting activated form GR has two principal mechanisms of action, transactivation and transrepression. A direct mechanism of action involves homodimerization of the receptor, translocation via active transport into the nucleus, and binding to specific DNA responsive elements activating gene transcription. This mechanism of action is referred to as transactivation. The biologic response depends on the cell type. In the absence of activated GR, other transcription factors such as NF-κB or AP-1 themselves are able to transactivate target genes. However activated GR can complex with these other transcription factors and prevent them from binding their target genes and hence repress the expression of genes that are normally upregulated by NF-κB or AP-1. This indirect mechanism of action is referred to as transrepression.

The GR is abnormal in familial glucocorticoid resistance. In the CNS, the glucocorticoid receptor is gaining interest as a novel representative of neuroendocrine integration, functioning as a major component of endocrine influence—specifically the stress response—upon the brain. The receptor is now implicated in both short and long-term adaptations seen in response to stressors and may be critical to the understanding of psychological disorders, including some or all subtypes of depression. Indeed, long-standing observations such as the mood dysregulations typical of Cushing's disease demonstrate the role of corticosteroids in regulating psychological state; recent advances have demonstrated interactions with norepinephrine and serotonin at the neural level. Dexamethasone is an agonist, and RU486 and cyproterone are antagonists of the GR. Also, progesterone and DHEA have antagonistic effects on the GR.

GCR Polymorphisms: Carriers of the 22-Glu-Lys-23 allele are relatively more resistant to the effects of glucocorticoids (GCs) with respect to the sensitivity of the adrenal feedback mechanism than non-carriers, resulting in a better metabolic health profile. Carriers have a better survival than non-carriers, as well as lower serum CRP levels. The 22-Glu-Lys-23 polymorphism is associated with a sex-specific, beneficial body composition at young-adult age, as well as greater muscle strength in males.

The REF SEQ ID (GRCh37.p5) is incorporated herein by reference.

TABLE 21
Novel SNPs in GCR pharmacogene exons that may impact drug response.

| | SNP | Position | MAF | SEQ ID NO: |
|---|---|---|---|---|
| A | AGCCTGAA A/G TATAAACAAAT | chr5: 142,720,722 | 2% | 65 |
| B | AACAATAG G/C ATAATGGAATG | chr5: 142,720,762 | 0.5% | 66 |
| C | AATGGAATGT T/G AAAGGAAAA | chr5: 142,720,775 | 1% | 67 |
| D | AGGAAAAC A/G AACCAATTTAAA | chr5: 142,720,787 | 1% | 68 |
| E | AGGCTTAGTA G/T GATCTGCTAA | chr5: 142,720,830 | 0.2% | 69 |
| F | TAACTCAGA A/G TCAGGAGTGTT | chr5: 142,720,846 | 5% | 70 |
| G | AAGGTCGG C/T ATTTAGCTGAAG | chr5: 142,750,206 | 0.4% | 71 |

Hydroxytryptamine Receptor 2A (HTR2A/5-HTR2A/Serotonin Receptor 2A)

HTR2A is a serotonin receptor. This is one of the several different receptors for 5-hydroxytryptamine (serotonin), a biogenic hormone that functions as a neurotransmitter, a hormone, and a mitogen. This receptor mediates its action by association with G proteins that activate a phosphatidylinositol-calcium second messenger system. This receptor is involved in tracheal smooth muscle contraction, bronchoconstriction, and control of aldosterone production. HTR2A receptors are located primarily in the neocortex, caudate nucleus, nucleus accumbens, olfactory tubercle, hippocampus and vascular and non-vascular smooth muscle cells. HTR2A receptors play a role in appetite control, thermoregulation and sleep. HTR2A receptors are also involved, along with various other 5-HT receptor populations, in cardiovascular function and muscle contraction. The human HTR2A receptor gene has been localized to chromosome 13 (13q14-q21).

HRT2A polymorphisms: HTR2A and antidepressant response: Several polymorphisms in the 5HT2A gene (−1438-G/A and 102-T/C in the promoter and His425Tyr in the coding region), display an association with treatment response to clozapine, as well as tardive dyskinesia. The strongest evidence for an association between an HTR2A SNP and selective serotoninergic re-uptake inhibitor (SSRI) antidepressant drug response is rs7997012, which is an intronic single nucleotide variant. In the STAR*D study, rs7997012 has been significantly associated with response to the SSRI drug citalopram, and other studies demonstrate significant association with fluoxetine. In patients diagnosed with generalized anxiety disorder, those who carried the HTR2A rs7997012 SNP G-allele have better treatment outcome over time in response to venlafaxine XR.

It is of interest to the differences reported in the 1000 Genomes Project with the results of the invention for the SNP rs7997012. A “scrubbed” version of the investigator's data showed that 2% of the so-called “AFRICAN (AFR)” population group had a G allele at this position, when actually none of the 7 different populations represented in the AFR sample had a G allele, based on close inspection of the excel spreadsheets.

TABLE 22
lists allele frequencies of SNP rs7997012.
ALLELE FREQUENCY OF SNP rs7997012

| | U.S. Population -17,131 genome sequences | | | |
|---|---|---|---|---|
| | Caucasians | Caucasians | African- | Asian- |
| | (White) | (Hispanic) | Americans | Americans |
| A/G | 55.83%/44.17% | 43.20%/56.8% | 3.47%/96.53% | 28.53%/71.47% |
| | Presumptive ‘Ancestral’ Genome Sequences from 1000 genomes project | | | |
| | EUROPEAN | AMERICAN | AFRICAN | ASIAN |
| A/G | 56%/44% | 68%/32% | 2%/98% | 24%/76% |

EUROPEAN: CEU Utah Residents (CEPH) with Northern and Western European ancestry; TSI: Toscani in Italia; FIN: Finnish in Finland; GBR: British in England and Scotland.
AMERICAN: MXL: Mexican Ancestry from Los Angeles USA; PUR: Puerto Rican from Puerto Rica; CLM: Colombian from Medellian, Colombia; PEL: Peruvian from Lima, Peru.
AFRICAN: YRI: Yoruba in Ibadan, Nigera; LWK: Luhya in Webuye, Kenya; GWD: Gambian in Western Divisons in The Gambia; MSL: Mende in Sierra Leone; ESN: Esan in Nigera; ASW: American's of African Ancestry in SW USA; ACB: African Carribean in Barbados
ASIAN: JPT: Japanese in Tokyo, Japan; CHB: Han Chinese in Beijing, China; CHB: Han Chinese in Bejing, China; CHS: Southern Han Chinese; CDX: Chinese Dai in Xishuanagbanna, China; KHV: Kinh in Ho Chi Minh City, Vietnam.

The SNP rs6311 is a rare variant of the human HTR2A gene that codes for the 5-HT2A receptor, and several studies have investigated the effect of the genetic variation on personality, e.g., personality traits measured with the Temperament and Character Inventory or with a psychological task measuring impulsive behavior. This SNP has also been investigated in rheumatology. Some research studies may refer to this gene variation as a C/T SNP, while others refer to it as a G/A polymorphism in the promoter region, thus writing it as, e.g., −1438 G/A or 1438G>A. Other important SNPs in HTR2A include rs6313, rs6314, and rs7997012.

The REF SEQ ID (GRCh37.p5) is incorporated herein by reference.

TABLE 23
Novel SNPs in HTR2A pharmacogene exons that may impact drug response.

| | | | | SEQ ID |
|---|---|---|---|---|
| | SNP | Position | MAF | NO: |
| A | CACCCTTCCT C/T ACTCACTTCCT | chr13: 47,439,301 | 0.5% | 72 |
| B | AGAAAGGCA G/A GACAAAATGAA | chr13: 47,439,535 | 1% | 73 |
| C | CCAAAAGTAA T/G CCAAAACAAA | chr13: 47,449,935 | 0.3% | 74 |
| D | CCATGACT G/A TTTTAAGAGGCTA | chr13: 47,459,966 | 0.7% | 75 |
| E | TTTTAGTTT G/C CTTATTCTCTCTGT | chr13: 47,460,040 | 0.7% | 76 |

HTR2C (Serotonin (5-Hydroxytryptamine, 5-HT) Receptor)

Serotonin, a neurotransmitter, elicits a wide array of physiological effects by binding to several receptor subtypes, including the 5-HT2 family of seven-transmembrane-spanning, G-protein-coupled receptors, which activate phospholipase C and D signaling pathways. This gene encodes the 2C subtype of serotonin receptor and its mRNA is subject to multiple RNA editing events, where genomically encoded adenosine residues are converted to inosines. RNA editing is predicted to alter amino acids within the second intracellular loop of the 5-HT2C receptor and generate receptor isoforms that differ in their ability to interact with G proteins and the activation of phospholipase C and D signaling cascades, thus modulating serotonergic neurotransmission in the CNS. The HTR2C gene spans 326,073 nucleotides on the X chromosome. Three transcript variants encoding two different isoforms have been found for this gene, as well as a microRNA that may alter transcriptional dynamics.

HTR2C polymorphisms: The SNP rs3813929, also known as −759C/T, has shown that patients with schizophrenia being treated with olanzapine reported a protective effect against weight-gain from the (T) allele of this SNP; with a rs3813929(T) allele corresponding to a body mass index increase of >=10% (p=0.002), whereas (C; C) homozygotes were not correlated with a protective effect against weight gain. This effect may also involve nearby SNP rs518147.

The REF SEQ ID (GRCh37.p5) is incorporated herein by reference.

TABLE 24
Novel SNPs in HTR2C pharmacogene exons that may impact drug response

| | | | | SEQ |
|---|---|---|---|---|
| | SNP | Position | MAF | ID NO: |
| A | TTCAGCCT G/A GATGACAGAAC | chrX: 113,981,588 | 4% | 77 |

NPY (Neuropeptide Y)

This gene encodes a neuropeptide that is widely expressed in the CNS and influences many physiological processes, including cortical excitability, stress response, food intake, circadian rhythms, and cardiovascular function. The neuropeptide functions through G protein-coupled receptors to inhibit adenylyl cyclase, activate mitogen-activated protein kinase (MAPK), regulate intracellular calcium levels, and activate potassium channels. A polymorphism in this gene resulting in a change of leucine 7 to proline in the signal peptide is associated with elevated cholesterol levels, higher alcohol consumption, and may be a risk factor for various metabolic and cardiovascular diseases. Most recently, several NPY SNPs have been strongly associated with risk for familial coronary artery disease (CAD). Family-based associations of NPY SNPs with CAD are presented in Table 25.

TABLE 25

| NPY SNP | PDT* | Geno-PDT |
|---|---|---|
| rs16147 | p = 0.05 | p = 0.03 |
| rs9785023 | p = 0.04 | p = 0.05 |
| rs5574 | p = 0.02 | p = 0.05 |
| rs16474 | p = 0.04 | p = 0.02 |
| rs16120 | p = 0.03 | p = 0.04 |

*Pedigree-Disequilibrium-Test

The REF SEQ ID (GRCh37.p5) is incorporated herein by reference.

TABLE 26
Novel SNPs in NPY pharmacogene exons that may impact drug response.

| | SNP | Position | MAF | SEQ ID NO: |
|---|---|---|---|---|
| A | CTTTGAAA G/T TTACAGCATTGTAGA | chr7: 24,327,620 | 1% | 78 |
| B | AGTACTGAAC T/C GGATGCAAG | chr7: 24,376,692 | 1% | 79 |

NTF3 (Neurotrophin 3)

The protein encoded by this gene, NT-3, is a neurotrophic factor in the NGF (Nerve Growth Factor) family of neurotrophins. It is a protein growth factor which has activity on certain neurons of the peripheral and central nervous system; it helps to support the survival and differentiation of existing neurons, and encourages the growth and differentiation of new neurons and synapses. NT-3 was the third neurotrophic factor to be characterized, after nerve growth factor (NGF) and BDNF (Brain Derived Neurotrophic Factor). NT-3 is unique in the number of neurons it can potentially stimulate, given its ability to activate two of the receptor tyrosine kinase neurotrophin receptors (TrkB and TrkC). Although a dinucleotide repeat has been found in one of the promoters of this gene, various SNPs have only been weakly linked to schizophrenia.

The REF SEQ ID (GRCh37.p5) is incorporated herein by reference.

TABLE 27
Novel SNPs in NT-3 pharmacogene exons that may impact drug response.

| | | | | SEQ ID |
|---|---|---|---|---|
| | SNP | Position | MAF | NO: |
| A | TGCCTGGCT G/A TGAAATTTCATTT | chr12: 5,568,755 | 0.5% | 80 |
| B | CGGATGTCCTAGA C/T GCAGGTTAT | chr12: 5,568,836 | 0.6% | 81 |
| C | CAAGTTTCC A/G TTCATTTTCTGCAT | chr12: 5,580,180 | 0.4% | 82 |
| D | ATTCAGCTTC A/G TGTTCTCTAACAT | chr12: 5,600,126 | 3% | 83 |

NTRK2

This gene encodes a member of the neurotrophic tyrosine receptor kinase (NTRK) family. This kinase is a membrane-bound receptor that, upon neurotrophin binding, phosphorylates itself and members of the MAPK pathway. Signaling through this kinase leads to cell differentiation. Alternate transcriptional splice variants encoding different isoforms have been found for this gene. In general, Trk (neurotrophin) receptors are single transmembrane catalytic receptors with intracellular tyrosine kinase activity. Trk receptors are coupled to the Ras, Cdc42/Rac/RhoG, MAPK, PI 3-K and PLCgamma signaling pathways. There are four members of the Trk family; TrkA, TrkB and TrkC and a related p75NTR receptor. p75NTR lacks tyrosine kinase activity and signals via NF-kappaB activation. Each family member binds different neurotrophins with varying affinities. TrkA potently binds nerve growth factor (NGF) and is involved in differentiation and survival of neurons and in control of gene expression of enzymes involved in neurotransmitter synthesis. TrkB has the highest affinity for brain-derived neurotrophic factor (BDNF) and is involved in neuronal plasticity, longterm potentiation and apoptosis of CNS neurons. TrkC is activated by neurotrophin-3 (NT-3) and is found on proprioceptive sensory neurons. p75NTR binds neurotrophin precursors with high affinity and retains low affinity to the mature cleaved forms. TrkA was originally identified as an oncogene as it is commonly mutated in cancers, particularly colon and thyroid carcinomas. A receptor tyrosine kinase is a “tyrosine kinase” which is located at the cellular membrane, and is activated by binding of a ligand to the receptor's extracellular domain. Other examples of tyrosine kinase receptors include the insulin receptor, the IGF1 receptor, the MuSK protein receptor, the Vascular Endothelial Growth Factor (or VEGF) receptor, etc.

The REF SEQ ID (GRCh37.p5) is incorporated herein by reference.

TABLE 28
Novel SNPs in NTRK2 pharmacogene exons that may impact drug response.

| | | | | SEQ ID |
|---|---|---|---|---|
| | SNP | Position | MAF | NO: |
| A | AAAGGGGCATA T/C ATTTATAAAAT | chr9: 87,550,028 | 0.4% | 84 |
| B | CAAGGACATAA A/T ATAGAGATATC | chr9: 87,460,980 | 0.7% | 85 |
| C | AGCTTCCAAG C/A TCAAGGAATTCT | chr9: 87,461,084 | 2% | 86 |
| D | CCAAAATAAT G/A GGTAATATATAT | chr9: 87,549,992 | 5% | 87 |
| E | TAGAAAGAAGTAG G/A GCATTGGCC | chr9: 87,499,996 | 0.7% | 88 |
| F | TCTCCATCTCCA G/A TGAGTATTGAG | chr9: 87,579,980 | 1% | 89 |
| G | GCCCAAG G/C ACATAAATAGAGAGAT | chr9: 87,460,973 | 0.6% | 90 |
| H | CAAAGAGAACTA A/G AAATTCCATGT | chr9: 87,609,978 | 3% | 91 |
| I | AGTAAATGTTCTC C/T CCTTCTGCAAG | chr9: 87,610,038* | 4% | 92 |
| J | GTTTTCCTAGA A/G CCTGTTACTTCAT | chr9: 87,620,027* | 0.9% | 93 |

*UCSC Genome Browser coordinates indicate different gene sequence, but that need to be corrected.

OPRM1

OPRMI (mu□opioid receptor, also known as OP3, MOP, MOR) is a member of the opioid family of G-protein-coupled receptors that also includes kappa, delta and NOP receptors. Three variants of the receptor designated mu1, mu2 and mu3 have been characterized, arising from the alternative splicing of this gene. Mu Opioid receptors are distributed throughout the neuraxis (neocortex, thalamus, nucleus accumbens, hippocampus, amygdala) and in the peripheral nervous system (myenteric neurons and vas deferens). The mu opioid receptor is the primary site of action for the most commonly used opioids, including morphine, heroin, fentanyl, and methadone. It is also the primary receptor for endogenous opioid peptides beta-endorphin and the enkephalins.

OPRM1 polymorphisms include rs1799971, rs2281617, rs510769 and rs9479757.

The rs1799971 SNP has been associated with nicotine dependence, alcoholism, and opiate abuse; rs2281617 and rs510769 have been associated with amphetamine abuse and rs9479757 has been associated with methadone abuse.

The REF SEQ ID (GRCh37.p5) is incorporated herein by reference.

TABLE 29
Novel SNPs in OPRM1 pharmacogene exons that may impact drug response.

| | SNP | Position | MAF | SEQ ID NO: |
|---|---|---|---|---|
| A | CCAGGGCTTT T/C GTTTATTGGGA | chr6: 154,387,541 | 0.6% | 94 |
| B | ACAAAAATTA G/T CCAGTGTGGTGGT | chr6: 154,394,992 | 5% | 95 |
| C | CCCTGGTAGAA T/G GTGCTTGACACA | chr6: 154,409,994 | 0.1% | 96 |

SLC6A2 (Solute Carrier Family 6 Member 2)

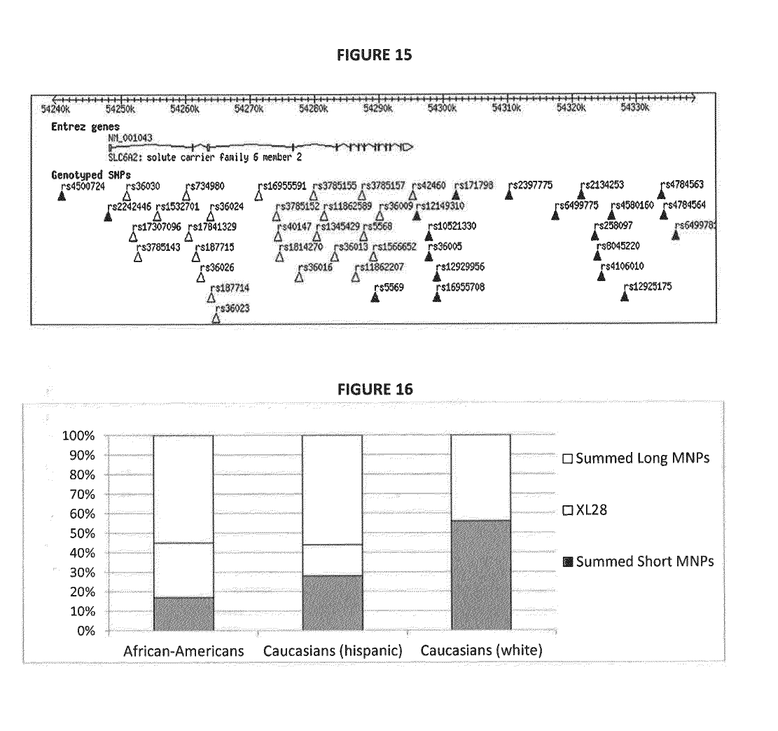

This gene encodes the norepinephrine transporter (NET) protein. It is a multi-pass membrane protein, which is responsible for reuptake of norepinephrine into presynaptic nerve terminals and is a regulator of norepinephrine homeostasis. SLC6A2 is located on human chromosome 16 locus 16q12.2. This gene is encoded by 14 exons. Based on the nucleotide and amino acid sequence, the NET transporter consists of 617 amino acids with 12 membrane-spanning domains. The structural organization of NET is highly homologous to other members of a sodium/chloride-dependent family of neurotransmitter transporters, including dopamine, epinephrine, serotonin and GABA transporters Mutations in this gene cause orthostatic intolerance, a syndrome characterized by lightheadedness, fatigue, altered mentation and syncope. Alternatively spliced transcript variants encoding different isoforms have been identified in the SLC6A2 gene. FIG. 15 depicts a number of identified SLC6A2 SNPs.

The REF SEQ ID (GRCh37.p5) is incorporated herein by reference.

TABLE 30
Novel SNPs in SLC6A2 pharmacogene exons that may impact drug response.

| | | | | SEQ |
|---|---|---|---|---|
| | SNP | Position | MAF | ID NO: |
| A | GTGCAGA G/T AGAGTTTGTGGAATC | chr16: 55,715,317 | 0.4% | 97 |
| B | GTGACCCTGCTT A/G GGATACCTAT | chr16: 55,730,266 | 3% | 98 |

SLC6A3 (Solute Carrier Family 6 Member 3)

This gene encodes the dopamine transporter protein, also known as DAT. DAT are sodium- and chloride-dependent members of the solute carrier family 6 (SLC6) widely distributed throughout the brain in areas of dopaminergic activity, including the striatum and substantia nigra. DAT proteins provide rapid clearance of dopamine, adrenaline and noradrenaline from the synaptic cleft, terminating the neurotransmitter signal. Dopamine transporters can also mediate an outward efflux and it has been suggested that inward and outward transport are independently regulated. Structural motifs include 12 transmembrane domains, extracellular loops, cytoplasmic C- and N-termini and putative phosphorylation sites. The 3′ UTR of this gene contains a 40 bp tandem repeat, referred to as a variable number tandem repeat or VNTR, which can be present in 3 to 11 copies. Variation in the number of repeats is associated with idiopathic epilepsy, attention-deficit hyperactivity disorder, dependence on alcohol and cocaine, susceptibility to Parkinson disease and protection against nicotine dependence.

The REF SEQ ID (GRCh37.p5) a is incorporated herein by reference.

TABLE 31
Novel SNPs in SLC6A3 pharmacogene exons that may impact drug response.

| | | | | SEQ |
|---|---|---|---|---|
| | SNP | Position | MAF | ID NO: |
| A | ATCATTCATCCA C/G CCATTCACCC | chr5: 1,419,224 | 1% | 99 |
| B | TCCCTGGGGCT T/C CCTGGGAGGCTT | chr5: 1,419,998 | 0.7% | 100 |
| C | AGGGAAATGTA G/A GTGTGAACAGG | chr5: 1,429,998 | 0.8% | 101 |
| D | ACGCAATGGG A/T GTTTTCTCCCTCG | chr5: 1,430,028 | 0.3% | 102 |
| E | GGGAGTTTTCT C/T CCTCGAGAATGT | chr5: 1,430,035 | 2% | 103 |
| F | AGGGCACCTCA G/C TAAAGTTCTCTT | chr5: 1,435,954 | 5% | 104 |
| G | TTAAACAAATCTA A/G GATCAGGAGT | chr5: 1,435,018 | 0.6% | 105 |
| H | CCTGTGCCAGA G/T CACAATGTATCT | chr5: 1,438,960 | 3% | 106 |
| I | ATCCCAAGGCTCTGA G/A CCCTCAGA | chr5: 1,439,038 | 0.6% | 107 |
| J | TCCACGGC A/G TGTCATGAACATGTT | chr5: 1,400,495 | 1% | 108 |
| K | GGCCCACAGGG C/T ACTGCTCCCGTG | chr5: 1,400,740 | 4% | 109 |
| L | AGCCCCCTGGG G/T GCTAAGAACACT | chr5: 1,400,960 | 0.8% | 110 |

SLC6A4 (Solute Carrier Family 6 Member 4)

This gene encodes the serotonin transporter, a membrane protein that takes up serotonin in pre-synaptic neurons. SLC6A4 is also known as SERT or 5-HTT, since serotonin is known chemically as 5-hydroxytryptamine. The main variants of the SLC6A4 gene that have been studied, however, are not SNPs—rather, they are short tandem repeats, also known as VNTRs (variable number tandem repeats). One such polymorphism is known as the 5-HTTLPR variant. Another polymorphism is the STin2 (intron 2) VNTR, which involves different alleles that correspond to 12-, 10-, 9-, or 7-repeat units of 17 bp. Both of these polymorphisms have been associated in some cases (but not others) with obsessive-compulsive disorder (OCD). Most recently, the STin2.12 carriers were reported to be at over 3× risk of OCD based on a study of ˜100 OCD patients.

The efficacy of commonly prescribed antidepressant drugs, such as paroxetine, has also been linked to SLC6A4 VNTR variants. A few other SNPs have been studied, including rs25531 and rs1042173, which has been implicated in heavier drinking alcoholics.

The REF SEQ ID (GRCh37.p5) is incorporated herein by reference.

TABLE 32
Novel SNPs in SLC6A4 pharmacogene exons that may impact drug response.

| | | | | SEQ |
|---|---|---|---|---|
| | SNP | Position | MAF | ID NO: |
| A | GCAGGACA G/A AAAGGATGATATAT | chr17: 28,543,194 | 3% | 111 |
| B | GGTCTTGACGCC T/C TTCCAGATGCT | chr17: 28,544,205 | 0.5% | 112 |
| C | GAAGAGCTGGG A/T TTGGCCTGTCC | chr17: 28,544,468 | 0.2% | 113 |
| D | AGTGTGCAGGTTA C/A TGATGCTGG | chr17: 28,549,558 | 0.6% | 114 |
| E | ACTGGGAGGGC C/A TGGCCGGGGCT | chr17: 28,550,010 | 1% | 115 |
| F | TTTGGACTTTAA A/T CCTATGGAATG | chr17: 28,550,136 | 2% | 116 |
| G | ACAGTTTGGGA G/C/T TTGAAATACG | chr17: 28,550,242 | 0.7% | 117 |
| H | GAGCAGAACCCC T/C CCCTGGTCCTTC | chr17: 28,559,034 | 4% | 118 |

### DEFINITIONS

As provided herein an allele is an alternative form of a gene (one member of a pair) that is located at a specific position on a specific chromosome. Alleles determine distinct traits that can be passed on from parents to offspring.

As provided herein allele frequency is the proportion of all copies of a gene that is made up of a particular gene variant (allele). In other words, it is the number of copies of a particular allele divided by the number of copies of all alleles at the genetic place (locus) in a population. It can be expressed for example as a percentage. In population genetics, allele frequencies are used to depict the amount of genetic diversity at the individual, population, and species level. It is also the relative proportion of all alleles of a gene that are of a designated type.

As provided herein analog refers to non-homologous genes that have descended convergently from an unrelated anscestor.

As provided herein the symbol/term*.bam/BAM is the compressed binary version of the Sequence Alignment/Map (SAM) format, a compact and index-able representation of nucleotide sequence alignments. Many next-generation sequencing and analysis tools work with SAM/BAM. For custom track display, the main advantage of indexed BAM over PSL and other human-readable alignment formats is that only the portions of the files needed to display a particular region are transferred.

As provided herein, the symbol/term*.bcl/BCL file type is primarily associated with ‘PDP-10’. The PDP-10 was a mainframe computer manufactured by Digital Equipment Corporation (DEC) from the late 1960s. It also used as a DNA sequence storage filr format.

As provided herein the term base, refers to the four chemical elements, represented by the letters A, G, G, T, which stand for adenine, cytosine, guanine, and thymine, that compose DNA.

As provided herein the term base pair refers to the linking between two nitrogenous bases on opposite complementary DNA or certain types of RNA strands that are connected via hydrogen bonds is called a base pair (often abbreviated bp). In the canonical Watson-Crick DNA base pairing, adenine (A) forms a base pair with thymine (T) and guanine (G) forms a base pair with cytosine (C). In RNA, thymine is replaced by uracil (U). As provided herein the term bioinformatics refers to Research, development, or application of computational tools and approaches for expanding the use of biological, medical, behavioral or health data, including those to acquire, store, organize, archive, analyze, or visualize such data.

As provided herein the term CPU refers to the central processing unit (CPU) is the portion of a computer system that carries out the instructions of a computer program, to perform the basic arithmetical, logical, and input/output operations of the system.

As provided herein the term CUDA refers to Compute Unified Device Architecture; A parallel computing platform and programming model invented by NVIDIA. It enables dramatic increases in computing performance by harnessing the power of the graphics processing unit (GPU).

As provided herein the term Endophenotype refers to a psychiatric concept and a special kind of biomarker. The purpose of the concept is to divide behavioral symptoms into more stable phenotypes with a clear genetic connection. The concept was originally borrowed by Gottesman & Shields from insect biology. Other terms with similar meaning but not stressing the genetic connection are “intermediate phenotype”, “biological marker”, “subclinical trait”, “vulnerability marker”, and “cognitive marker”.

As provided herein the term Exon refers to a protein-coding component of a gene.

As provided herein the symbol/term*.fasta/FASTA format (in bioinformatics) refers to a text-based format for representing either nucleotide sequences or peptide sequences, in which nucleotides or amino acids are represented using single-letter codes. The format also allows for sequence names and comments to precede the sequences. The format originates from the FASTA software package, but has now become a standard in the field of bioinformatics. It is especially useful for variant analysis software such as SIFT and PolyPhen.

As provided herein the genome of eukaryotes is contained in a single, haploid set of chromosomes. The human genome is made up of approximately 23,000 genes, or three billion chemical base pairs.

As provided herein the term Genotype refers to a gene for a particular character or trait may exist in two allelic forms; one is dominant (e.g. A) and the other is recessive (e.g. a). Based on this, there could be three possible genotypes for a particular character: AA (homozygous dominant), Aa (heterozygous), and aa (homozygous recessive).

As provided herein the term Genotyping refers to the measurement of genetic variation between species members.

As provided herein the term Genotypic frequency refers to the frequency of a genotype—homozygous recessive, homozygous dominant, or heterozygous—in a population. If you don't know the frequency of the recessive allele, you can calculate it if you know the frequency of individuals with the recessive phenotype (their genotype must be homozygous recessive).

As provided herein the term Graphics Processing Unit (GPU) refers to a programmable logic chip that performs parallel operations on graphics data. In GPU-clusters, they perform parallel operations on multiple sets of data, being used as vector processors for a variety of applications that require repetitive computations which allows specified functions from a normal C program to run on the GPU's stream processors. This makes C programs capable of taking advantage of a GPU's ability to operate on large matrices in parallel, while still making use of the CPU when appropriate.

As provided herein the term Homology refers to a trait or any characteristic of organisms that is derived from a common ancestor.

As provided herein the term Introns refers to intervening sequence that interrupt protein coding sequence of a gene. Non-coding portions of precursor mRNA, removed before mature RNA formed. Introns are spliced out of the resulting mRNA sequence is exons ready to be translated into proteins.

As provided herein the term KB versus Kb versus Kbit-KB: that is close to 2¹⁰, or 1,024 bytes. As provided herein the term Kilo (in science) means 10⁴, or one thousand. As provided herein the term Kb (in genomics) means one thousand bases. Kbp means one thousand base pairs. As provided herein the term Kbit (in computer science) means 1,024 bits, that is, equal to 2¹⁰ bits. Often used as a measure of transmission speed between different computer devices.

As provided herein the term MB versus Mb versus Mbit-MB: means megabyte in computer science that is used to describe a measure that is close to 2²⁰, or 1,048,576 bytes. Often used to describe storage of data. As provided herein the term Mega (in science) means 106, or one million. As provided herein the term Mb (in genomics) means one million bases. As provided herein the term Mbit (in computer science) means 1,048,576 (that is, 2²⁰) bits. Often used as a measure of transmission speed between different computer devices.

As provided herein the term Minor Allele Frequency (MAF) means that within a population, SNPs can be assigned a minor allele frequency—the ratio of chromosomes in the population carrying the less common variant to those with the more common variant. It is important to note that there are variations between human populations, so a SNP allele that is common in one geographical or ethnic group may be much rarer in another. With the advent of modern bioinformatics and a better understanding of evolution, this definition is no longer necessary.

As provided herein the term Multiple nucleotide polymorphisms (MNP) refers to alleles of common length >1, for example AAA/TTT.

As provided herein the term Next-generation DNA sequencing (NGS) refers to massively parallel DNA-sequencing technologies that produce many hundreds of thousands or millions of short reads (25-500 bp) for a low cost and in a short time.

As provided herein the term Orthologs refers to a homologus series that have evolved from common ancestor by speciation. They are assumed to have evolved to perform similar function.

As provided herein the term Paralog refers to Homologous sequences separated by a gene duplication event. They have evolved to perform different functions.

As provided herein the term Pharmacodynamic gene refers to genes that encode proteins that impact biochemical and physiological effects of drugs on the body or on microorganisms or parasites within or on the body, as well as and the mechanisms of drug action and the relationship between drug concentration and effects.

As provided herein the term Pharmacogene refers to any gene that encodes a protein that is involved in pharmacodynamics or pharmacokinetics, or other physiological processes, whose polymorphic variations are associated with drug efficacy or toxicity.

As provided herein the term Pharmacogenomics refers to the study of variations of deoxyribonucleic acid (DNA) and ribonucleic acid (RNA) characteristics as related to drug response. A pharmacogenomic test is intended to identify inter-individual variations in whole-genomes or candidate genes, single-nucleotide polymorphisms, haplotype markers, or alterations in gene expression that may be correlated with pharmacological function and therapeutic response. In pharmacogenomics, researchers are able to look at variations in all the genes in a group of individuals simultaneously to determine the basis for variations in drug response.

As provided herein the term Pharmacogenetics refers to the study of variations in DNA sequence as related to drug response.

As provided herein the term Phenotype (from Greek phainein, ‘to show’+typos, ‘type’) refers to the composite of an organism's observable characteristics or traits. These characteristics can be controlled by genes, by the environment, or a combination of both.

As provided herein the term Polymorphism refers to the occurrence in a population of several phenotypic forms due to differences in gene sequences at particular alleles.

As provided herein the term PolyPhen-Polymorphism Phenotyping (PolyPhen) refers to a tool which predicts possible impact of an amino acid substitution on the structure and function of a human protein. Open source software.

As provided herein the term Promoter (in genetics) refers to a region of DNA that facilitates the transcription of a particular gene. Promoters are located near the genes they regulate, on the same strand and typically upstream (towards the 5′ region of the sense strand).

As provided herein the term Reference Sequence refers to the NCBI Reference Sequence Project (RefSeq) is an effort to provide the best single collection of naturally occurring genomes, in this case, the human genome. The latest release is 52, as of Mar. 5, 2012.

As provided herein the term Resequencing is used for determining a change in DNA sequence from a “reference” sequence, followed by sequencing. The resultant sequence is compared to a reference or a normal sample to detect mutations.

As provided herein the term Single nucleotide polymorphisms (SNPs) refers to the most common type of genetic variation among people. Each SNP represents a difference in a single DNA nucleotide. For example, a SNP may replace the nucleotide cytosine (C) with the nucleotide thymine (T) in a certain stretch of DNA.

As provided herein the term Sorting Intolerant From Tolerant (SIFT) predicts whether an amino acid substitution affects protein function using sequence conservation and other features. SIFT is often applied to nonsynonymous variants and laboratory-induced missense mutations. Open source software

As provided herein the symbol/term*.tar—The TAR (“tarball”) refers to the file format initially developed to write data to sequential I/O devices for tape backup purposes. It is now commonly used to collect many files into one larger file for distribution or archiving, while preserving file system information such as user and group permissions, dates, and directory structures. It is the whole human genome output file from Complete Genomics, Inc.

As provided herein the symbol/term*.tiff—The phrases “Tagged Image File Format” and “Tag Image File Format” were used as the subtitle to some early versions of the TIFF specification; it is commonly used as a graphics file format, but also is the major raw read output of the Illumina DNA sequencing machines.

As provided herein the term Xenologs refers to homologs resulting from horizontal gene transfer between two organisms.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1

### Validation of Results of Analysis of 24 Selected Pharmacogenes in 17,131 Whole Genomes

Table 33 shows the process for the validation of SNPs and MNPs:

| Concordance and Error-Checking | Invention | Standard and References | |
|---|---|---|---|
| Cross-platform concordance | Aggregation Module of | College of American Pathology | |
| of novel and known SNPs and | invention | standard for reference | |
| MNPs between Illumina, Life | | laboratories; | |
| Technologies and Complete | | A. | SINNOTT JA AND KRAFT |
| Genomics | | | P. HUM GENET. 2012 |
| | | | JANUARY; 131(1): 111-9. |
| | | B. | Bansal V et al. Genome |
| | | | Research, 2010, Vol. 20, pp. |
| | | | 537-545. |
| Statistical correction for Type | Multi-Genome Variant | A. | Fox P et al. Nat Methods 5: |
| 1 errors | Module of invention | | 183-188. |
| | | B. | Muralidharan O et al. Nucleic |
| | | | Acids Research, (Nov. |
| | | | 7, 2011) 2012, Vol. 40, No. 1 |
| | | | e5 doi: 10.1093/nar/gkr851. |
| | | C. | Yang F and Thomas D C. Hum |
| | | | Heredity, Jul. 2, |
| | | | 2011; 71: 209-220. |
| | | D. | Tintle T et al. Genet |
| | | | Epidemiol. 2011; 35 (Suppl |
| | | | 1): S56-S60. |
| Statistical strategy for | Multi-Genome Variant | A. | Su Z et al. Expert Rev Mol |
| validation of SNPs and MNPs | Module of invention | | Diagn. 2011 April; 11(3): 333- |
| through replication runs, | | | 43. |
| checking against reference | | B. | Li, H et al. Genome Research |
| genome for known | | | 18, 1851-1858 (2008) |
| polymorphisms, and | | | |
| specificity testing of rare | | | |
| variants. | | | |

### Example 2

### Example of Novel MNPs of a Pharmacogene Implicated in Antidepressant Drug Response in Psychiatry that Show Racial Subpopulation MNP Heterogeneity

The 5-HTTLPR promoter of the SLC6A4 pharmacogene displays racial subpopulation differences as described in Table 34:

| Characteristics | | | | | Population Frequency | | |
|---|---|---|---|---|---|---|---|
| | | Known | | | African- | Caucasians | Caucasians |
| | | SNP: | | | Americans = | (hispanics) = | (whites) = |
| MNP | Length | rs25531 | #TFBS | GC | 2,866 genomes | 5,313 genomes | 9,204 genomes |
| L_{A} | 528 | A | 151 | − | 8% | 8% | 10% |
| L_{G} | 528 | G | 151 | − | 5% | 12% | 11% |
| XL_{16A} | 528 | A | 122 | − | 5% | 6% | 5% |
| XL_{16B} | 534 | A | 98 | − | 3% | — | 5% |
| XL_{16C} | 528 | A | 112 | − | 5% | 5% | — |
| XL_{16D} | 529 | G | 110 | − | 5% | 1% | — |
| XL_{16E} | 547 | A | 110 | − | 5% | 7% | — |
| XL_{16F} | 529 | A | 149 | − | 5% | — | — |
| XL₁₇ | 551 | A | 160 | − | 5% | 12% | 10% |
| XL₁₈ | 574 | A | 173 | − | 5% | 3% | 3% |
| XL₁₉ | 598 | A | 170 | − | 2% | 2% | — |
| XL₂₀ | 610 | A | 177 | − | 1% | — | — |
| XL₂₂ | 655 | A | 177 | − | 1% | — | — |
| XL₂₈ | 752 | A | 211 | + | 28% | 16% | — |
| S_{A} | 465 | A | 18 | − | 11% | 8% | 12% |
| S_{G} | 465 | G | 18 | − | 6% | 10% | 9% |
| XS₁₁ | 419 | G | 2 | − | — | 7% | 12% |
| XS_{14A} | 486 | G | 4 | − | — | — | 6% |
| XS_{14B} | 487 | G | 4 | − | — | 3% | 5% |
| XS_{14C} | 487 | G | 6 | − | — | — | 7% |
| XS_{14D} | 441 | — | — | − | — | — | 5% |

FIG. 16 shows the comparison of the 5-HTTLPR MNPs in the SLC6A4 gene across racial subpopulations.

### Example 3

### Novel XL₂₈ MNP Sequence Found in the 5-HTTLPR Promoter of the SLC6A4 Gene in 17,131 Whole Human Genomes by the Present Invention, that Contains a Canonical Glucocorticoid Receptor Binding Motif and Shows Ethnic Diversity

AF126506.1 & XL₂

Length=752 bp

Query 112

SEQ ID NO: 119 shows the large number of Variable Number Tandem Repeats (VNTRs), and the Canonical glucocorticoid receptor binding site (underlined). The sequence is located in the 5′-HTTLPR promoter, which does not encode protein.

(SEQ ID NO: 119)
5′CCTGCATCCTGCACCCCCAGGCATCCCCCCTGCAGCCCCCCCAGCATCCCCCCTGCAGCC
CCCCCAGAACAGGGTGTTTCCCCCCCTGCAGCCCCCCCAGCATCCCCCCTGCAGCCCCCCCAGCAT
CCCCCCTGCAGCCCCCCCAGCATCTCCCCTGCACCCCCAGCATCCCCCCTGCAGCCCTTCCAGCATC
CCCCTGCACCTCTCCAGGATCTCCCTGCAACCCCCATTATCCCCCCTGCACCCCTCGCAGTATCCC
CCCTGCACCCCCCAGCATCCCCCCATGCAACCCCCGGCATCCAGCATTCTCCTTGCACCCTACCAG
TATTCCCCCGCATCCCGGCCCCCCTGCACCCCTCCAGCATTCTCCTTGCACCCTACCAGTATTCCC
CCGCATCCCGGCCTCCAAGCCTCCCGCCCACCTTGCGGTCCCCGCCCTGGCGTCTAGGTGGCACCA
GAATCCCTCCAAGCCTCCCGCCCACCTTGCGGTCCCCGCCCTGGCGTCTAGGTGGCACCAGAATCC
CGCGCGGACTCCACCCGCTGGGAGCTGCCCTCGCTTGCCCGTGGTTGTCCAGCTCAGTCCCGCGCG
GACTCCACCCGCTGGGAGCTGCCCTCGCCGGACTCCACCCGCTGGGAGCTGCCCTCGCCTCCAAGC
CTCCCGCCCACCTTGCGGTCCCCTAGGTGGCACCAGAATCCCTCCAAGCCTCCCGCCCACCTTGCG
GTCCCCGCCCTGGCGTCTAGGTGGCACCTCC-3′

### Example 4

### Novel Polymorphisms Associated with Pharmacogene-Mediated Antidepressant Response in Posttraumatic Stress Disorder (PTSD)

A. ADCYAP1R1

A novel MNP removes an estrogen responsive element found in the gene, which correlates with antidepressant drug response in female patients with posttraumatic stress disorder (PTSD) (Table 36).

TABLE 36

| | |
|---|---|
| Canonical Estrogen Responsive Element: | GGTCAnnnTGxCCt (SEQ ID NO: 120) |

Coordinate 31135504 of ADYCYAP1R1

| | |
|---|---|
| SNP rs2267735 (known variant) | GGTCAc/gagaGgaCg (SEQ ID NO: 121) |
| Novel MNP variant found at same position | TTTTCGACCCCCCC (SEQ ID NO: 122) |
| 12% of female Caucasians (white) | |

B. CRHR1

A novel SNP interrupts putative glucocorticoid receptor binding site, as defined in association studies by known SNPs (Table 37).

TABLE 37
Coordinate 43871147 of CRHR1

| | |
|---|---|
| SNP rs12944712 (known variant) | AGGAGACCTGG/AGGTTGGAGCT |
| | (SEQ ID NO: 123) |
| Novel intronic SNP interrupts putative | AGG/AAGACCTGG/AGGTTGGAGCT |
| glucocorticoid receptor binding site | (SEQ ID NO: 124) |

C. SLC6A4

A novel MNP adds canonical glucocorticoid receptor binding site to the degenerate 5-HTTLPR of the SLC6A3 gene, which encodes the serotonin transporter gene with a frequency of 28% in African-Americans and 16% of Caucasians (hispanic), but not Caucasians (white). This promoter has 37 different MNPs in the pooled genome DNA. This promoter has been associated with psychotropic drug response in hundreds of articles, and is known to be glucocorticoid regulated in L (long) forms of the degenerate sequence. However, this was the first time a putative GCR canonical motif had been found in this pharmacogene. (See, Table 38).

TABLE 38

| Canonical glucocorticoid receptor | | AGAACAtcccTGTACA | |
|---|---|---|---|
| binding site | | (SEQ ID NO: 125) | |
| Gene Promoter/ | | Canonical | Fold activation by |
| Intron | Protein | sequence | Dexamethasone |
| DBI | diazepam binding inhibitor | AGAACAttgGGTTTC | 2.3 ± 0.4 |
| | | (SEQ ID NO: 126) | |
| Tat | tyrosine aminotransferase | | 22.3 ± 4.6 |
| UGT8 | UDP glycosyltransferase 8 | AGAACAtttTGTACG | 8.2 ± 10 |
| | | (SEQ ID NO: 127) | |
| FKBP5 | FK506-binding protein 5 | AGAACAgggTGTTCT | 5.9 ± 0.4 |
| | | (SEQ ID NO: 128) | |
| 5′-HTTLPR- | serotonin transporter | AGAACAgggTGTTTC | Unknown, but 6-12 |
| Variant XL₂₈ | protein | (SEQ ID NO: 129) | fold increases in L |
| | | | MNPs in cell culture |

What is claimed is:

1. A method for interrogating thousands of aggregated whole human genome sequences, the method comprising (a) using a targeted analysis of one or more selected pharmacogenes and (b) determining polymorphic sequences that may associate with a drug response, wherein the method is executed on an inexpensive, energy-efficient, and heterogeneous graphics processing unit (GPU)-cluster based workstation.

2. The method of claim 1, comprising the steps of (a) aggregating and performing a concordance check on populations of completed whole genome DNA sequences; (b) scanning assembled whole human genomes for target enrichment of one or more selected pharmacogenes, wherein said scanning is performed by using genome browser coordinates for the one or more selected pharmacogenes based on user input; (c) applying a multi-genome variant analysis algorithm to identify gene variants in said one or more pharmacogenes; (d) optionally, applying an algorithm to identify a potentially deleterious mutation that could impact a drug response; and (e) detecting a single nucleotide polymorphism (SNP), a multi-nucleotide polymorphism (MNP) or both SNP and MNP, but not other structural variants, and applying a statistical error-checking method to validate the SNP, MNP, or both SNP and MNP having allele frequencies of 0.1% to 99%.

3. The method of claim 1, wherein the one or more selected pharmacogenes comprises one or more genes selected from the group consisting of the ABCB1 gene, the ADCYAP1R1 gene, the ADRA2A gene, the BDNF gene, the COMT gene, the CRHBP gene, the CRHR1 gene, the DBI gene, the DRD2 gene, the DRD4 gene, the FKBP5 gene, the GCR gene, the HTR2A gene, the HTR2C gene, the NPY gene, the NT3 gene, the NTRK2 gene, the OPRM1 gene, the SLC6A2 gene, the SLC6A3 gene, and the SLCA4 gene.

4. The method of claim 3, wherein the SNP, MNP, or both SNP and MNP is selected from one or more of the polymorphisms identified in SEQ ID NOs: 1-15 (gene: ABCB1), 16 (ADCYAPIR1), 17-18 (ADRA2A), 19-20 (BDNF), 21-23 (COMT), 24 (CRHBP), 25-28 (CRHR1), 29-46 (DBI), 47-51 (DRD2), 52-54 (DRD4), 55-64 (FKBP5), 65-71 (GCR), 72-76 (HTR2A), 77 (HTR2C), 78-79 (NPY), 80-83 (NT3), 84-93 (NTRK2), 94-96 (OPRM1), 97-98 (SLC6A2), 99-110 (SLC6A3), and 111-118 (SLC6A4).

5. A method for determining likelihood of an adverse or modified response to an anti-depressant or psychiatric drug in a patient in need thereof, the method comprising obtaining a biological sample from said patient and assaying the biological sample for the presence at least one polymorphism in one or more pharmacogenes selected from those identified in SEQ ID NOs: 1-118, wherein the presence of at least one polymorphism indicates that an adverse or modified response to the anti-depressant or psychiatric drug is likely.

6. The method of claim 5, wherein the anti-depressant or psychiatric drug is selected from the group consisting of clozapine, fluvoxamine, escitalopram, paroxetine, amitriptyline, venlafaxine, citalopram, risperidone, nortriptyline, fluoxetine, olanzapine, tricyclic antidepressants, selective serotonin reuptake inhibitors, mitrtazapine, oxymetazoline, clonidine, epinephrine, norepinephrine, phenylephrine, dopamine, p-synephrine, p-tyramine, serotonin, p-octopamine, yohimbine, phentolamine, mianserine, chlorpromazine, spiperone, prazosin, propranolol, alprenolol, and pindolol.

7. An isolated nucleic acid consisting of any one of the sequences identified by SEQ ID NOs: 1-118.

8. The isolated nucleic acid of claim 7, wherein the nucleic acid is a cDNA.

9. A vector comprising the isolated nucleic acid of claim 7.

10. A cell comprising the isolated nucleic acid of claim 7.
